(19) United States
(12) Patent Application Publication
(10) Pub. No.: US 2023/0203585 A1
(43) Pub. Date: Jun. 29, 2023

(54) EPIGENETIC MODIFICATION OF SELECT GENES AS MARKERS OF HYPERSOMNOLENCE

(71) Applicant: Wisconsin Alumni Research Foundation, Madison, WI (US)

(72) Inventors: David Thomas Plante, Madison, WI (US); Reid Spencer Alisch, Prairie du Sac, WI (US)

(73) Assignee: Wisconsin Alumni Research Foundation, Madison, WI (US)

(21) Appl. No.: 17/926,804

(22) PCT Filed: May 21, 2021
(86) PCT No.: PCT/US2021/033677

§ 371 Date: Nov. 21, 2022

Related U.S. Application Data
(60) Provisional application No. 63/029,127, filed on May 22, 2020.

(51) Int. Cl. C12Q 1/6883

(52) U.S. Cl. CPC C12Q 1/6883; C12Q 2600/154

(57) ABSTRACT

Differentially methylated positions associated with hypersomnolence, idiopathic hypersomnia, and long sleep time are provided herein along with biomarker panels thereof and methods of diagnosing and providing a prognosis of hypersomnolence, idiopathic hypersomnia, and long sleep time using said differentially methylated positions.

### CROSS-REFERENCE TO RELATED APPLICATIONS

The present application is a national filing under 35 U.S.C. § 371 of PCT/US2021/033677, filed May 21, 2021, which claims priority to U.S. Application 63/029,127, filed May 22, 2020, the content of each of which is incorporated herein by reference in its entirety.

### SEQUENCE LISTING

The instant application contains a Sequence Listing which has been submitted in XML format and is hereby incorporated by reference in its entirety. The XML copy, created on Nov. 21, 2022, is named USPTO-221121-APP-P200207US02-SEQ_LIST.xml and is 70,668 bytes in size.

### STATEMENT REGARDING FEDERALLY SPONSORED RESEARCH

This invention was made with government support under T42 OH008672 awarded by the Center for Disease Control and Prevention. The government has certain rights in the invention.

### FIELD OF THE INVENTION

The invention is directed to tools and methods for diagnosing and providing a prognosis of hypersomnolence, idiopathic hypersomnia, and long sleep time using differentially methylated positions.

### BACKGROUND

Disorders of unexplained hypersomnolence are a significant cause of dysfunction and reduced quality of life (Khan and Trotti. Chest. 2015, 148(1):262-273). Much of the research into disorders such as idiopathic hypersomnia (IH) has focused on symptoms of chronic daytime sleepiness and/or impaired quality of wakefulness. However, excessive quantities of sleep that are nonrestorative contribute equally if not more to the impairing aspects of these disorders. Currently, the pathophysiology that underlies excessive sleep duration (i.e., hypersomnia) in disorders such as IH remains unknown and is a vital area of investigation for sleep medicine.

A key step to begin to understand the biological causes of hypersomnia requires examination of factors that contribute to habitual sleep duration more broadly. While the specific determinants of habitual sleep time remain unknown, recent genome wide association studies (GWAS) have highlighted the role of the paired box 8 (PAX8) gene in human sleep duration. PAX8 likely serves several roles as a master transcription factor crucial to embryonic development and maintenance functions after birth (Fernandez et al. Nat Rev Endocrinol. 2015, 11(1):29-42). Using both self-report and accelerometer data in population-based cohorts, single nucleotide polymorphisms (SNPs) in PAX8 and its related antisense gene (PAX8 AS1) have repeatedly demonstrated significant effects on habitual sleep duration (Gottlieb et al. Mol Psychiatry. 2015, 20(10):1232-1239; Lane et al. Nat Genet. 2017, 49(2):274-281; Dashti et al. Nat Commun. 2019, 10(1):1100; Jones et al. PLoS Genet. 2016, 12(8):e1006125; Jones et al. Nat Commun. 2019, 10(1):1585). However, the SNPs themselves impact habitual sleep duration only marginally (e.g., only a few minutes longer sleep per day). Thus, while GWAS investigations have pointed to a role of PAX8 in sleep duration, specific sequence variability in the PAX8 gene is unlikely to be a major contributing factor in pathological hypersomnia.

While GWAS help identify potential genes linked to phenotypic traits, additional methods are needed to help diagnose and treat patients and examine specific identified genes that relate to the pathophysiology of human disease.

### SUMMARY OF THE INVENTION

One aspect of the invention is directed to a method comprising isolating target DNA from a biofluid sample from a subject to obtain isolated target DNA, treating the isolated target DNA with bisulfite to generate bisulfite modified DNA, amplifying the bisulfite modified DNA with primers specific to one or more human genomic positions to generate amplified DNA, and quantifying methylation at the one or more human genomic positions in the amplified DNA.

In some embodiments, the one or more human genomic positions comprise 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, 20 or more, 21 or more, 22 or more, 23 or more, 24 or more, 25 or more, 26 or more, or each of 113992930 of chromosome 2, 113993052 of chromosome 2, 113993142 of chromosome 2, 113993304 of chromosome 2, 113993313 of chromosome 2, 113994035 of chromosome 2, 113994578 of chromosome 2, 113995370 of chromosome 2, 113995456 of chromosome 2, 124985406 of chromosome 9, 124985756 of chromosome 9, 124987896 of chromosome 9, 124988720 of chromosome 9, 124989052 of chromosome 9, 124989241 of chromosome 9, 124989294 of chromosome 9, 124989337 of chromosome 9, 124989408 of chromosome 9, 124989550 of chromosome 9, 124989839 of chromosome 9, 46391392 of chromosome 4, 46391436 of chromosome 4, 46391448 of chromosome 4, 46391476 of chromosome 4, 46391524 of chromosome 4, 46391532 of chromosome 4, and 46391694 of chromosome 4.

In some embodiments, the one or more human genomic positions comprise one or more, two or more, three or more, four or more, five or more, six or more, seven or more, eight or more, or each of 113992930 of chromosome 2, 113993052 of chromosome 2, 113993142 of chromosome 2, 113993304 of chromosome 2, 113993313 of chromosome 2, 113994035 of chromosome 2, 113994578 of chromosome 2, 113995370 of chromosome 2, and 113995456 of chromosome 2.

In some embodiments, the one or more human genomic positions comprise one or more, two or more, three or more, four or more, five or more, or each of 113993142 of chromosome 2, 113993304 of chromosome 2, 113993313 of chromosome 2, 113994578 of chromosome 2, 113995370 of chromosome 2, and 113995456 of chromosome 2.

In some embodiments, the one or more human genomic positions comprise one or more, two or more, or each of 113994578 of chromosome 2, 113995370 of chromosome 2, and 113995456 of chromosome 2.

In some embodiments, the one or more human genomic positions comprise 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, or each of 124985406 of chromosome 9, 124985756 of chromosome 9, 124987896 of chromosome 9, 124988720 of chromosome 9, 124989052 of chromosome 9, 124989241 of chromosome 9, 124989294 of chromosome 9, 124989337 of chromosome 9, 124989408 of chromosome 9, 124989550 of chromosome 9, 124989839 of chromosome 9, 46391392 of chromosome 4, 46391436 of chromosome 4, 46391448 of chromosome 4, 46391476 of chromosome 4, 46391524 of chromosome 4, 46391532 of chromosome 4, and 46391694 of chromosome 4.

In some embodiments, the one or more human genomic positions comprise one or more, two or more, three or more, four or more, five or more, six or more, seven or more, eight or more, nine or more, ten or more, or each of 124985406 of chromosome 9, 124985756 of chromosome 9, 124987896 of chromosome 9, 124988720 of chromosome 9, 124989052 of chromosome 9, 124989241 of chromosome 9, 124989294 of chromosome 9, 124989337 of chromosome 9, 124989408 of chromosome 9, 124989550 of chromosome 9, and 124989839 of chromosome 9.

In some embodiments, the one or more human genomic positions comprise one or more, two or more, three or more, four or more, five or more, six or more, or each of 46391392 of chromosome 4, 46391436 of chromosome 4, 46391448 of chromosome 4, 46391476 of chromosome 4, 46391524 of chromosome 4, 46391532 of chromosome 4, and 46391694 of chromosome 4.

In some embodiments, the primers consist of fewer than 60 total primers, fewer than 54 total primers, fewer than 48 total primers, fewer than 42 total primers, fewer than 36 total primers, fewer than 30 total primers, fewer than 24 total primers, fewer than 20 total primers, fewer than 18 total primers, fewer than 16 total primers, fewer than 14 total primers, fewer than 12 total primers, fewer than 10 total primers, fewer than 8 total primers, fewer than 6 total primers, fewer than 4 total primers, or fewer than 2 total primers.

In some embodiments, the primers comprise one or more pairs of primers. In some embodiments, each pair of primers amplifies a DNA fragment of 100-400 bp, 150-350 bp, or 200-300 bp in length.

In some embodiments, the one or more pairs of primers consists of fewer than 30 pairs of primers, fewer than 25 pairs of primers, fewer than 20 pairs of primers, fewer than 18 pairs of primers, fewer than 16 pairs of primers, fewer than 14 pairs of primers, fewer than 12 pairs of primers, fewer than 10 pairs of primers, fewer than 9 pairs of primers, fewer than 8 pairs of primers, fewer than 7 pairs of primers, fewer than 6 pairs of primers, fewer than 5 pairs of primers, fewer than 4 pairs of primers, fewer than 3 pairs of primers, fewer than 2 pairs of primers, or 1 pair of primers.

In some embodiments, the biofluid is selected from the group consisting of saliva, blood, cerebrospinal fluid, sweat, tears, semen, and urine. In some embodiments, the biofluid is blood or saliva.

In some embodiments, the subject is a human.

In some embodiments, the biofluid sample is from a healthy subject.

In some embodiments, the biofluid sample is from a subject with a mean sleep latency as measured by a multiple sleep latency test of less than 10 minutes.

In some embodiments, the biofluid sample is from a subject with a pupillary unrest index as measured by infrared pupillometry of at least 9.8.

In some embodiments, the biofluid sample is from a subject demonstrating lapses in a psychomotor vigilance test of at least 4.8.

In some embodiments, the biofluid sample is from a subject previously diagnosed with diabetes, asthma, or hypertension.

In some embodiments, the quantifying provides a degree of methylation at the one or more human genomic positions in the amplified DNA. In some embodiments, the degree of methylation at the one or more human genomic positions indicates total sleep time in the subject. In some embodiments, the degree of methylation at the one or more human genomic positions indicates hypersomnolence in the subject. The degree of methylation for any one the one or more human genomic position in any one of the aforementioned embodiments can be as shown in Tables 1 and 2.

In some embodiments, such as embodiments in which the degree of methylation at the one or more human genomic positions indicates hypersomnolence in the subject, the method further comprises administering to the subject a wake promoting medication. In some embodiments, the wake promoting medication is an amphetamine or modafinil. In some embodiments, such as embodiments in which the degree of methylation at the one or more human genomic positions indicates hypersomnolence in the subject, the method further comprises administering to the subject an oxybate or derivative thereof. In some embodiments, such as embodiments in which the degree of methylation at the one or more human genomic positions indicates hypersomnolence in the subject, the method further comprises having the subject undergo sleep extension therapy.

Another aspect of the invention is directed to a method of amplification, such as a method of amplifying a differentially methylated position (DMP). In some embodiments, the method comprises providing a reaction mixture comprising bisulfite modified target DNA from a biofluid sample from a subject and primers specific to one or more human genomic positions, heating the reaction mixture to a first predetermined temperature for a first predetermined time, cooling the reaction mixture to a second predetermined temperature for a second predetermined time under conditions to allow the primers to hybridize with complementary sequences on the bisulfite modified target DNA, and repeating the heating and the cooling, wherein an amplified target DNA sample is formed.

The one or more human genomic positions can comprise any of the one or more human genomic positions as described above in any combination.

In some embodiments, the primers consist of fewer than 60 total primers, fewer than 54 total primers, fewer than 48 total primers, fewer than 42 total primers, fewer than 36 total primers, fewer than 30 total primers, fewer than 24 total primers, fewer than 20 total primers, fewer than 18 total primers, fewer than 16 total primers, fewer than 14 total primers, fewer than 12 total primers, fewer than 10 total primers, fewer than 8 total primers, fewer than 6 total primers, fewer than 4 total primers, or fewer than 2 total primers.

In some embodiments, the primers comprise one or more pairs of primers. In some embodiments, each pair of primers amplifies a DNA fragment of 100-400 bp, 150-350 bp, or 200-300 bp in length.

In some embodiments, the one or more pairs of primers consists of fewer than 30 pairs of primers, fewer than 25 pairs of primers, fewer than 20 pairs of primers, fewer than 18 pairs of primers, fewer than 16 pairs of primers, fewer than 14 pairs of primers, fewer than 12 pairs of primers, fewer than 10 pairs of primers, fewer than 9 pairs of primers, fewer than 8 pairs of primers, fewer than 7 pairs of primers, fewer than 6 pairs of primers, fewer than 5 pairs of primers, fewer than 4 pairs of primers, fewer than 3 pairs of primers, fewer than 2 pairs of primers, or 1 pair of primers.

In some embodiments, the biofluid is selected from the group consisting of saliva, blood, cerebrospinal fluid, sweat, tears, semen, and urine. In some embodiments, the biofluid is blood or saliva, in some embodiments, the subject is a human.

In some embodiments, the reaction mixture additionally comprises a polymerase and free nucleotides comprising adenine, thymine, cytosine, and guanine. In some embodiments, the reaction mixture additionally comprises a reaction buffer and MgCl₂. In some embodiments, at least one of the primers is biotinylated.

Another aspect of the invention is directed to a biomarker panel. In some embodiments, the biomarker panel comprises a probe, primers, or a combination thereof specific to one or more human genomic positions. The one or more human genomic positions can comprise any of the one or more human genomic positions as described above in any combination.

In some embodiments, the primers consist of fewer than 60 total primers, fewer than 54 total primers, fewer than 48 total primers, fewer than 42 total primers, fewer than 36 total primers, fewer than 30 total primers, fewer than 24 total primers, fewer than 20 total primers, fewer than 18 total primers, fewer than 16 total primers, fewer than 14 total primers, fewer than 12 total primers, fewer than 10 total primers, fewer than 8 total primers, fewer than 6 total primers, fewer than 4 total primers, or fewer than 2 total primers.

In some embodiments, the primers comprise one or more pairs of primers. In some embodiments, each pair of primers amplifies a DNA fragment of 100-400 bp, 150-350 bp, or 200-300 bp in length.

In some embodiments, the one or more pairs of primers consists of fewer than 30 pairs of primers, fewer than 25 pairs of primers, fewer than 20 pairs of primers, fewer than 18 pairs of primers, fewer than 16 pairs of primers, fewer than 14 pairs of primers, fewer than 12 pairs of primers, fewer than 10 pairs of primers, fewer than 9 pairs of primers, fewer than 8 pairs of primers, fewer than 7 pairs of primers, fewer than 6 pairs of primers, fewer than 5 pairs of primers, fewer than 4 pairs of primers, fewer than 3 pairs of primers, fewer than 2 pairs of primers, or 1 pair of primers.

In some embodiments, the biomarker panel comprises a probe specific to each of the one or more human genomic positions.

In some embodiments, the biomarker panel comprises probes and the probes consist of fewer than 28 probes.

In some embodiments, either the probes or the primers are arrayed on a substrate.

In some embodiments, the substrate is selected from the group consisting of a chip, a bead, a plate, a microfluidic device, or a multiwall plate.

In some embodiments, the biomarker panel comprises: a probe specific to each of the one or more human genomic positions; primers specific to each of the one or more human genomic positions; and a carrier. In some embodiments, the carrier is a substrate. In some embodiments, the substrate is selected from the group consisting of a chip, a bead, a plate, a microfluidic device, or a multiwall plate. In some embodiments, the probes are arrayed on the substrate. In some embodiments, the carrier is a buffered solution.

### BRIEF DESCRIPTION OF THE DRAWINGS

The patent or patent application file contains at least one drawing in color. Copies of this patent or patent application publication with color drawings will be provided by the Office upon request and payment of the necessary fee.

FIGS. 1A-1C show quantification of hypersomnolence variables. A) 3D-scatterplot of, MSLT mean sleep latency (MSL), pupillary unrest index (PUI), and PVT lapses in full data sample (N=28). B) Correlation (r) matrix for all hypersomnolence variables (p>0.2 for all associations). C) Histogram of ad libitum PSG TST (mean 8.7±1.9 hours; range 5.8 to 13.9 hours).

FIG. 2 shows relative positions of PSG-associated differentially methylated positions at the PAX8 and PAX-AS1 loci. Schematic of PAX8 and its antisense gene PAX8-AS1. The relative positions of probes measuring methylation levels of CpG sites annotated to PAX8 and PAX8 AS1 with their genomic 5′-3′positions are provided (inset panel; x-axis) vs. the −log10 of the adjusted local index of significance (aLIS) P-value (y-axis). All probes were tested for positive correlations with PSG (red dots) and negative PSG (black dots) sleep duration. The level of aLIS P-values<0.05 (black line) is indicated. Dotted blue box denotes cluster of 5 CpG sites near PAX8 AS1 promoter. The inset table provides the Illumina provided CpG ID number (CpG ID), the CpG chromosome (chr) number and genomic position, and the correlation R matrix between DNA methylation levels and total sleep duration for these 5 CpG sites.

FIG. 3 shows average percent methylation of 5 DMPs in PAX8-AS1 promoter stratified by total sleep time above and below 9 hours. Average methylation was 8.6% greater in those with excessive sleep duration at this threshold (77.4% vs. 68.8%; P-value=0.026).

### INCORPORATION BY REFERENCE

All publications, patents, and patent applications mentioned in this specification are herein incorporated by reference to the same extent as if each individual publication, patent, and patent application was specifically and individually indicated to be incorporated by reference.

### DETAILED DESCRIPTION OF THE INVENTION

Subjects with unexplained hypersomnolence have differentially methylated genes that are implicated in idiopathic hypersomnia and hypersomnolence associated conditions.

The present disclosure describes biofluid based assays for the diagnosis, prognosis, or therapeutic treatment recommendation of hypersomnolence, idiopathic hypersomnia, and acute or chronic long sleep time in a subject. The present disclosure describes differentially methylated positions (D1VIPs) associated with the paired box 8 (PAX8) gene and the paired box 8 anti-sense gene (PAX8-AS1) that are characteristic of hypersomnolence, idiopathic hypersomnia, and acute or chronic long sleep time in a subject. Also described are DMPs associated with LIM/homeobox protein 6 (LHX6) and gamma-aminobutyric acid receptor subunit alpha-2 (GABRA2). These characteristic biomarkers may be used to assay methylation in DNA isolated from a biofluid sample from a subject. These characteristic biomarkers may be used in the development of a screening panel, a resequencing panel, or a diagnostic kit for the processing of DNA isolated from a biofluid sample from a subject.

As used herein, “hypersomnolence” refers to excessive daytime sleepiness often accompanied by prolonged sleep duration. Biological and physiological causes of hypersomnolence are not universally known, many subjects may receive an ambiguous diagnosis despite comprehensive evaluation. Hypersomnolence is associated with a number of clinical phenotypes including, but not limited to reduced mean sleep latency (MSL), increased total sleep time (TST), increased pupillary unrest index (PUI), and increased lapses on a psychomotor vigilance test (PVT). Methods for measuring each of the hypersomnolence-associated phenotypes are known in the art.

In some embodiments, the hypersomnolence-associated phenotype is reduced MSL. MSL may be measured using a multiple sleep latency test in which quantifies sleep propensity during repeated nap opportunities and the presence of sleep onset rapid eye movement (REM) periods. In some embodiments, MSL measurements associated with hypersomnolence are less than 5 minutes for pathogenic sleepiness, and less than 8-10 minutes for moderate sleepiness.

In some embodiments, the hypersomnolence-associated phenotype is increased TST. TST may be measured using ad libitum polysomnography. In some embodiments, TST associated with hypersomnolence is greater than 9 hours. In more severe forms it is greater than 10 or 11 hours.

In some embodiments, the hypersomnolence-associated phenotype is increased PUI. PUI may be measured using infrared pupillometry. In some embodiments, PUI associated with hypersomnolence is at least 9.8 (unitless).

In some embodiments, the hypersomnolence-associated phenotype is lapses in the PVT. PVT is a well-validated measure used in sleep research to quantify the ability to sustain attention and response in a timely manner to salient signals. A PVT lapse is generally characterized as a failure to respond to the given stimuli within a predetermined time (typically 500 msec). In some embodiments, PVT lapses associated with hypersomnolence is at least 4.8 lapses.

As used herein, “idiopathic hypersomnia” refers to excessive daytime sleepiness even after a full or prolonged night's sleep. A subject with idiopathic hypersomnia may sleep normally or for long periods during the night but is still excessively sleepy during the day, which may cause dangerous situations if sleepiness is brought on during driving, working, or other activities. Napping generally does not relieve the sleepiness of an individual with idiopathic hypersomnia.

As used herein, “long sleep time” refers to acute or chronic periods of sleep by an individual that are longer than about 9 hours for an adult human, about 10 hours for a teenaged human, or about 12 hours for a young child.

Described herein are differentially methylated positions associated with the PAX8 and PAX8-AS1 genes that are characteristic of hypersomnolence, idiopathic hypersomnia, and long sleep time in a subject. Also described herein are differentially methylated positions associated with LHX6 that are characteristic of mean sleep latency and GABRA2 that are characteristic of pupillary unrest index.

As used herein, “differentially methylated positions” or “DMP” refers to CpG dinucleotide positions with a significant increase (hypermethylation), a significant decrease (hypomethylation) in methylation (e.g., composite of 5-methylcytosine +5-hydroxymethylation [5mC+5hmC]) relative to hypersomnolence clinical phenotypes, or a significant change in methylation that correlates to a hypersomnolence clinical phenotype. In some embodiments, the DMP corresponds to a position with a change in methylation that correlates to a hypersomnolence clinical phenotype as outlined in Table 1 and Table 2.

TABLE 1
Differentially methylated positions within PAX8 and PAX8-AS1 associated with total sleep time.

| | | Genomic | DNA | Relation to a | | aLIS | | DMP | DNAm |
|---|---|---|---|---|---|---|---|---|---|
| CpG ID | Chromosome | position | strand¹ | CpG Island² | UCSC RefGene Name | P-value³ | Correlation⁴ | Status | cutoff |
| cg21550016 | chr2 | 113992930 | + | N_Shore | PAX8; LOC440839; LOC654433 | 0.04313874 | 0.3592121 | Hyper | >0.7 |
| cg07772999 | chr2 | 113993052 | + | N_Shore | PAX8; LOC440839; LOC654433 | 0.03748023 | 0.2653631 | Hyper | >0.7 |
| cg19083407 | chr2 | 113993142 | − | N_Shore | PAX8; LOC440839; LOC654433 | 0.02880542 | 0.4265851 | Hyper | >0.7 |
| cg07594247 | chr2 | 113993304 | − | Island | PAX8; LOC440839; LOC654433 | 0.02598576 | 0.1988143 | Hyper | >0.7 |
| cg17445212 | chr2 | 113993313 | − | Island | PAX8; LOC440839; LOC654433 | 0.02179952 | 0.3193101 | Hyper | >0.7 |
| cg21610815 | chr2 | 113994035 | + | Island | PAX8; LOC654433; LOC440839 | 0.01861881 | 0.1549738 | Hyper | >0.8 |
| cg00422909 | chr2 | 113994578 | − | S_Shore | PAX8-AS1; PAX8 | 0.02050616 | 0.3243021 | Hyper | >0.6 |
| cg13020245 | chr2 | 113995370 | + | S_Shore | PAX8-AS1; PAX8 | 0.02841775 | 0.01047076 | Hyper | >0.8 |
| cg04345118 | chr2 | 113995456 | + | S_Shore | PAX8; LOC654433; LOC440839 | 0.04720507 | −0.0790697 | Hypo | <0.7 |

¹(+) sense and (−) antisense DNA strands
²CpG island relationships are described in detail above
³The R package NHMMfdr was used to detect the adjusted local index of significance (aLIS), an extension of adjusted P-values, for each probe by first converting all P-values to z-scores, followed by employing a Hidden Markov Model to determine the “aLIS P-value.”
⁴Correlation R matrix between DNA methylation levels and hypersomnolence variables

TABLE 2
Differentially methylated positions within other DMP-associated genes.

| | | | | | UCSC | | | | | |
|---|---|---|---|---|---|---|---|---|---|---|
| | | Genomic | DNA | Relation to a | RefGene | aLIS | | DMP | Associated | DNAm |
| CpG ID | Chromosome | position | strand¹ | CpG Island² | Name | P-value³ | Correlation⁴ | Status | Phenotype | cutoff |
| cg10264903 | chr9 | 124985406 | − | N_Shelf | LHX6 | 0.04040517 | 0.2371739 | Hyper | MSLT | <0.15 |
| cg02825412 | chr9 | 124985756 | − | N_Shore | LHX6 | 0.02633677 | 0.0431312 | Hyper | MSLT | <0.68 |
| cg09723488 | chr9 | 124987896 | + | Island | LHX6 | 0.02187027 | −0.2624312 | Hypo | MSLT | >0.7 |
| cg04282082 | chr9 | 124988720 | − | Island | LHX6 | 0.01565797 | −0.2763055 | Hypo | MSLT | >0.65 |
| cg11479503 | chr9 | 124989052 | + | Island | LHX6 | 0.0181288 | −0.186529 | Hypo | MSLT | >0.7 |
| cg21213617 | chr9 | 124989241 | + | Island | LHX6 | 0.02273288 | −0.1609523 | Hypo | MSLT | >0.7 |
| cg21469772 | chr9 | 124989294 | − | Island | LHX6 | 0.02100529 | −0.2001834 | Hypo | MSLT | >0.7 |
| cg13571460 | chr9 | 124989337 | + | Island | LHX6 | 0.02199509 | −0.1841574 | Hypo | MSLT | >0.65 |
| cg05136264 | chr9 | 124989408 | − | Island | LHX6 | 0.02387765 | −0.1936458 | Hypo | MSLT | >0.7 |
| cg05037505 | chr9 | 124989550 | − | Island | LHX6 | 0.02907199 | −0.2365778 | Hypo | MSLT | >0.6 |
| cg15124400 | chr9 | 124989839 | + | Island | LHX6 | 0.03946631 | −0.2126105 | Hypo | MSLT | >0.7 |
| cg25145765 | chr4 | 46391392 | − | N_Shore | GABRA2 | 0.03552697 | −0.1922142 | Hypo | PUI | <0.03 |
| cg02620694 | chr4 | 46391436 | + | N_Shore | GABRA2 | 0.01720495 | −0.3900152 | Hypo | PUI | <0.03 |
| cg14778074 | chr4 | 46391448 | + | N_Shore | GABRA2 | 0.01414029 | −0.2889577 | Hypo | PUI | <0.09 |
| cg02633148 | chr4 | 46391476 | + | N_Shore | GABRA2 | 0.01611223 | 0.1642397 | Hyper | PUI | >0.06 |
| cg08206959 | chr4 | 46391524 | + | N_Shore | GABRA2 | 0.01240927 | −0.2945528 | Hypo | PUI | <0.05 |
| cg14411873 | chr4 | 46391532 | + | N_Shore | GABRA2 | 0.01317009 | −0.4174173 | Hypo | PUI | <0.06 |
| cg12205729 | chr4 | 46391694 | + | N_Shore | GABRA2 | 0.02400582 | −0.3520862 | Hypo | PUI | <0.02 |

¹(+) sense and (−) antisense DNA strands
²CpG island relationships are described in detail above
³The R package NHMMfdr was used to detect the adjusted local index of significance (aLIS), an extension of adjusted P-values, for each probe by first converting all P-values to z-scores, followed by employing a Hidden Markov Model to determine the “aLIS P-value.”
⁴Correlation R matrix between DNA methylation levels and hypersomnolence variables

As used herein, “significant change” refers to a change (i.e., increase or a decrease) with a statistical signification of aLIS P-value<0.05 when compared to hypersomnolence clinical phenotype.

As used herein, “significant increase” refers to an increase with a statistical signification of aLIS P-value<0.05 when compared to hypersomnolence clinical phenotype.

As used herein, “significant decrease” refers to a decrease with a statistical significance of aLIS P-value<0.05 when compared to hypersomnolence clinical phenotype.

As used herein, “differentially methylated position-associated genes” or “DMP-associated genes” refers to the genes in which the DMPs are located or the genes with which the DMPs are most closely associated. In some embodiments, the DMP may be in the coding region of the DMP-associated gene. In some embodiments, the DMP may be in the promoter region of the DMP-associated gene. In some embodiments, the DMP-associated genes are PAX8, PAX8-AS1, LHX6, and GABRA2.

As used herein, “CpG island” refers to short CpG-rich areas defined as >0.5 kb stretches of DNA with a G+C content greater than or equal to 55% and an observed:expected frequency of at least 0.6. In general, CpG are located in the promoter region and 5′ to the transcription start site, although a CpG island may occur anywhere in a gene. Many terms of art are associated with characterization of CpG sites and differential methylation relative to a particular gene. A CpG shore refers to a region with lower CpG density than a CpG island that lies within the 2 kb up- and downstream of a CpG island. A CpG shelf refer to the 2 kb outside of a CpG shore. A CpG canyon refers to regions of low methylation more than 3.5 kb distant from CpG islands and CpG shores that frequently contain transcription factors. A CpG ocean refers to regions with low methylation and not characterized as either a CpG island, CpG shore, CpG shelf, or CpG canyon. A gene body refers to the entire gene from transcription start site to the end of the transcript. A gene desert refers to regions with very few, if any, genes in a 500 kb region. See, for example, Asmar et al. (DNA methylation and hydroxymethylation in cancer, Epigenetic Cancer Therapy, 2015, p. 9-30) which is incorporated herein by reference in its entirety.

DMP biomarker candidates associated with PAX8 and PAX8-AS1 show significant (aLIS P-value<0.05) association between degree of methylation in target regions and total sleep time measured with ad libitum polysomnography when DNA samples from subjects with hypersomnolence are analyzed. DMP biomarkers associated with PAX8 and PAX8-AS1 are recited in Table 1.

In some embodiments, the CpG sites of interest within an about 400 base pair region (SEQ ID NO:1) located in the coding region of the PAX8 gene and near the promoter of the PAX8-AS1 gene. All DMPs in this about 400 base pair region are hypermethylated (i.e., increasing methylation is associated with longer sleep time). In some embodiments, the CpG sites of interest are within the region defined by SEQ ID NO:2.

(CpG DMPs indicated in BOLD, the ~400 bp
region of SEQ ID NO: 1 is in italics):
SEQ ID NO: 2 [symbol]
CCGCCATAGCTGCATGGCCCCGGGACCTCCCTGTCGTACCTGAGAGGA
GGGCCTGGCCCGTGAACTGCCCGTACACGGAGGCAGCATGGGGAAAGGCA
TTGAAGGGCGGGACCCCGGAGC [symbol] ACTTGCTGCAGATCCAAAAAGGCGGA
GCTAGATAAAGAGGAAGGGGTGGAGCTAGAACTGGACACCTCGGGGGTTT
CCTGCTTTATGG [symbol] AAGGGTGAGTGAGGATCTGCCGGAGGGAGGGAGACA
ACAAGGAGAGAGGGGTGTGAGATGGCGGGGAGGGAACACGCACAAGCCAA
GCCGTGGGATGTGGAGGGTGCGGGCGGGGCGCGGGGCAGGCTCAGCTGCC
CTCAGAGTCTGGGCTGGGGAGAGC [symbol] GGGCCCA [symbol] AGGCGTGGCAAGGCG
GGGAGAGAGAAGCTAGACCTCCCTGCTGCGCTTCTGCAGCGAAAATGGAG
AGACCCGGAAGGCGTGTGTGCGCCCGCCTCTCCCACAGGAGGGAGCGCGG
AGACCCGGGAGCGGCCTAGGACCGGAGGCGCGACCCCTCGGCCCACCTTG
AGGCCCGGCCTAGGACCGGAGGCGCGACCCCTGGGCCCACCTTGAGGCCC
GGCCTAGGACTGGAGGCGCGACCCCTCGGCCCACCTTGCGGGAGCCGCCT
AGGACCGGAGGCGCGACCCCTCGGCCCAGCTTGAGGCCCGGCCTAGGACC
GGAGGCGCGACCCCTCGGCCCACCTTGCGGGAGCCGCCTAGGACCGGAGG
CGCGACCCCTCGGCCCACCTTGAGGCCCGGCCTAGGACCGGAGGCGCGAC
CCCTGGGCCCACCTTGAGGCCCGGCCTAGGACTGGAGGCGCGACCCCTCG
GCCCACCTTACGGGAGCCGCCTAGGGCCGGAGGCGCGACCCCTCGGCCCA
GCTTGAGGCCCGGCCTAGGACCGGAGGCGCGACCCCTCGGCCCACCTTGA
GGCCCGGCCTAGGACTGGAGGCGCGACCCCTCGGCCCACCTTGAGGCCCG
GCCTAGGACCGGAGGCGCGACCCCTGGGCCCACCTTGAGGCCCGGCCTAG
GACTGGAGGCGCGACCCCTCGGCCCACCTTGAGGCCCGGCCTAGGACCGG
AGGCGCGACCCCTGGGCCCACCTGGCGGCCCGGCCGGCACAGCCCGCCTC
TCCTCTCCAGGCCAGGGCCCCACACCTTCCGCCTGACAGCCAGCCAAGCT
CTTCAGTCCCCCGCCCTCCACCTGCCAGGGAGGCTCCGGGCGTTGTACCT
GCCACCACGGGGTAGGTCTGGTGAGTCGAGAGGTTGCGCCCCAGTGGCGT
GTTGGAAGGGGTCAGGGTGGCCTTCCCGTCGTCCAGGGTGCTGTTGAGCA
AGGGCAGCGGGTAGAGGCCCTGGGGAGCAAAGAGAAGTCAGCGCACAGAG
ACGATCCAACAAGCCGACCTTCCTGCAGACCCTGAGCCTGTGTCTTAGGA
CTTGGCAGCCCTCATCTCCCCAGGAGAGGTCCTCATCGCCCCCTTCTTCC
TTTACGTTCTCAACTCCACTCGGCACGTTTCGTTCATGCTCATTGTCCTC
CCGCATCCCTGGAATTCAAGGCCAGCTGGTAGCATGGGTGCCCAGACGTG
GGATAGAGAGTCTCAGCCAGAGGACCACACCCTGGTTGGATGGCTGGTCG
GCTTCCTGGTTTCACCACACTGTTGGGACCCACAGAATGCAGGACGGTGC
CAAGGACTGGAGCAGTCTCCAACCGGACTGTTCAGGGTCCTTGCATCTGT
TAATTTATTCTTCCCCAGATAGGAAACCCAGGCACAAAGGTTTAAGAAAC
TTGTCCCACTTCCAGATGGGCCAAAGATAGGATTCAGATTCGAGGGATCA
GAATGTGTCTGCTCTCAACCACTGCCCTAAACTGGGTGACATAAGGACCA
CTTCTTACATTTGCAAAGAGCTTATATCAGAGCTTTGCCTCTGTTGTCCT
AGTAGAGTCCCCCAGCAGCCCAGTGACATGGGTGAGTAATATTATGTCTG
TGTTTTATATGTGGAGCCTGGAACTCAGGTAAATGGCCCAATTTTGTATA
GTGAAACAGACTAAACCCGGGATTTTTCGGCTGTGGACCCAGGTGCTTTA
CCCACCACGTGGATTTCTTCTTTTCTCTCTCCACATCCAAACAAATAGAA
GTGTGCAAGTTCGGCTTGAAAATGGTATTGAGAGTTGTTTTTGTTGGAAA
CACTCTTTGTCAAGTACCTGGGGGTCTTCATCAGTAAGACCTCATTTTAG
ATGCCTCCTGGCCTCCCCTTCCCAGGAGCACAGCTATGACCTTAGGTACT
CCTTCCGAAAAGAACTTGTTTAACTAAAGGTAAGTGTACCTCATCCTCAC
CATGGCCTCCTTCCACTGGGGAAGCAGATAGCGCAGAAAAAAGAACACAC
CCATTCCCCACATACCTTCACACTCGTCACATACCTGCTACGTGAGATGT
GCAAAGCTGAATTCAGGGAATGCTCAGTAGTTACATAACAGTGCCACTAA
AGGCAATTGTTTTCAGTGATTTCCATCG

DMP biomarker candidates associated with LHX6 show significant (aLIS P-value<0.05) association between degree of methylation in target regions and mean sleep latency measured by multiple sleep latency test when DNA samples from subjects with hypersomnolence are analyzed. DMP biomarkers associated with LHX6 are recited in Table 2.

(CpG DMPs indicated in BOLD, the top
candidate region of SEQ ID NO: 4
is in italics)
SEQ ID NO: 3
CGATCGGCTTTTTCTTTCCCTTTTTGTGGGTTTTATTATTTTTGTTACAG
GTGGGGCTGCCAGGGATGGGTTATGCCTGCAGGGGGAGAAATGATCAGAA
GAGCCACAATTCTACCCTCAACCCAGGGAATGTGAGCTTTTACTGCCTCC
ATTTTCTTAACCAGAAATCTGGTTTAGTTTTGCTTTCAGGGCTCTTGTCA
TTGGAAAAAGATTAGTGCTCAGAACCTGCAGCCCAGAAATGGCCCAGTCC
CATCCCTCCAGGCAGGCTCTGCTCCTCAAGCTGCGTGGGGAAATGCCATC
ATGGGCTTGTGCCCAGCATCCACCCTGCCAATGATGACAACAGTGATAAT
CGAGGCATTCATACCAACCACCATTTAGGGAGCCTTTATAGAGACTTCCC
CCCAACAGTGGCCTCTTTGGCAGCTCTGGGAGACAGCCTTGGAAAGGGCG
AACTAAGGGCCGAGAGAAGCAGCAACTTGCGAAGGTCACTACTGGCCAGA
CTGGGCCCAGTCTCCTGCCTCTTCCCACAGTGGCTGCACACCCTACTCTC
TACCCCATTGCAGTAGGAAGAGAACTCCAGAATAAGGGGAGGCTCTGAAC
AGTGGAGTCAGGATCCGTGTTTTGACTAGTTTCAAGGTCTAGTAAAGCAC
AGTATCCAAGGAGGCTCAGTCCAGCATGGGCATGGGCTAGGGCTGGAGAA
GTAGAAAAGAAATTTGGGCAAGTAGTGAATAAGACTGCCAAATCTATTGT
TAGGAGAGCCAGGAGAGGTGTCCTAGTGGTGCCTTAGTCTATGGGAGAGA
CCCAGACACCATTCTGCTGGGTTCCCAAGCTGGCACCTTAAATTTAGTAG
CTGATGAATCCAAGGAGCCCCCAAGCATCATCTGGCATGTTTTTTTGTAT
GTTTGTTTGTTTGTTTTTTGAGACAGAGTCTCTCTCTGTTACCCAGGCTG
GAGTGTAGTGACGTGATCTTGGCTCACCACAATGTCCGCCTCCCAGGTTC
AAGTGATTCTCTTGCCTCAGCCTCCTGAGTAGCTGGGACTACAGGCACGT
GCCACCACACCTGGCTAAGTTTTGTATTTTTAGTAGAGATGGGTTTCACC
ATGTTGGCCAGGATGGTCTCGATCTCCTGACCTTGTGATCTACCAGCCTT
GGCCTCCCAAAGTGCTGCGATTATAGGTGTGAGCCACCACACCCGGCCCA
TCTGGCATGTTTTATACCAACTGGAGTCAGGCCTAGAGCAGGGAGGTGGC
TAGACAGTGACTGTAACCTCCATTTCCTGTCCTGGCCAAACCAATACTCA
GATGGGACAGTGAGAGGAGACATCACACTCTGGTTACACACCATTAACCC
CTGTCTGACTACACACACACACACACACAGCCGTCCAGATTCACAAATAC
GTTCTTATCACCCCCTTCATCCCTCAGTGCCTGGGTGGAGGCCCTTAAGC
AGGAATGCCCCCTCAGAGGAGAGGGTGGGAGTGGAATGATGGGATTTCAG
AATCCCCTCACCTCACCCTCTCACCTCCAGATCCTAGTCCAGATGTAATC
CTGTCCTCACCACTTTGCGTGCACGTGCACGCACACACACATGCACACAC
ACAGTCTCAACATTCACATACAGACACCCACTCTTGACACCCACACATGA
ACTAAAAGACAAACGTTGGTCCATAAAGGAACACACATGCTATCTACATA
CCCCCAACCCTGCCCAGTGATGCTTTCCCCAGCACTACTGCACAGATCCC
AGACAATACAGACACACAGGCACTCCTTGCACACTGAGTCCTAGGCCACT
TGGTTGAGCTGCCCCCACCCCACCCCAGCTGCACACTCTTCTACATCCAT
CCCATAAAGGAAGAGTCCGTCTCTTTAACCATGCTTCTCATGGTTTTCAG
AGTACCCTTGCCCCAAATGCTGCCAACCTCCCCCTAGACTCAAGATCTTC
TCTTTCCAGATCGCTGGAGGCTCAATGCCAGTGCACAGGAGGAGATCGCC
TTCCACCATATTCCCTGACTCCCAGAGGCTCTTTCTAGGGCAGGGGTCTG
GCCAGGAAGACTGAAGCCATGACGTCTTCCTGTGCAAGGATAGGAAAGAT
GTCTGTGGGTTGGAGGGTAGTTATAGGGAAGCAGCTTTCTCCACTTTTTG
AGAAAGCACTTGGGAACAAGCATCCCTCTCAGGAAGTGATTCTCCACCAT
TCTCCTCTGATCCTATAGGCCTGGTGGTCCAAGCCTCAGGCCAGTTGATG
CCTCCCACTGGGATCAGCAGACAAGGCCATGACCCTGCAGTGACCTCGAC
CCCATAATCAGGTCAGGTCCGGGCTTCATGGCTAGAAGCGAGGCCCAGGC
GAGTGGCCAAGACGCGGCAGGACAGGAGGATGCGCCTGGCTGCTCTCGGC
TGCTGGACGCGGCCTGCAGTAGGGAGAGCGCAGCCCGGGGTAATCCCACT
CAGGGCTCCGGGTGCCCAGTTCTCCCACTTCCCATCCTGGCGGCCTAAAG
GCAAGGCCTAGACCCTGGAGAGGCCAAAGTCGGGCCAAAACAACAGCGAA
GCTGGGACTGGAACAGGCGCTGGGGGCCTGCGCATGACCCCAGGCTCAGA
ATGCGACACTCCAGTGACGCCGGGGAATGAGTTGAGATGGAGCCGGAGAC
AGAGGTTGGGGTTCAGGACGAGGCGCTTGGGTATGGGATCGGATCTGAGA
CGGTTGCGGCGCTGGTGCTTTGGGCTGGACCAGACTCGAACCCAGAGCGG
AGCGAGGGTCAGAGCCAGGGCAAGAGTCCGAAGCCCAGCTCAGCGTCCAG
CCCGCGAGAGACCCCACCATGAATGACGTCCGAGTAGGGTTGGAACTCGA
AGCCCGAACGCACTGGAAATGCAGCCGGACGACGGCTGGGATCCGAGTGG
GTCCGGGCCAGAAATGGGGACCTCAGAGCTCCACCGAGGCGCTCGAGGTC
AGAACTGAAGCCTGAGACTTAGGCAGGACCCGAGACAGAGCCAGAGACGA
TACCGAAACCCAATGGACCGCGAGGACCGAAGGCAGATCCGGGGCGCAAA
CCTGTGCAGGCACTGGCGCGCTGCCTGGAGCTCTGGGTTCGCGCCGCTGA
GCGCCGGCAGGTTGGACCAGCGCTGCGCGCCGGAAGTGCACTCGGGACGC
CGGGGTTCTGGAGGAGAGACCACACGCCGCAAAAGTGGCCTCCGAATGCG
CCCGGGGCCTGCCCTCGGCGACACTGCTGGCTCGGCGGCCGCAGCCTACC
TGAAGTAGTCCATCTTGCAGAAGATCTCCTTGTTCTTGATGTAGCAGCTG
TTCTGCTGCCTCAGCGACGTGCGACACACGGAGCACTCGAGGCACCGCAC
GTGCCAGATGAGGTTGTTGACCTGGGGACGGGGCGGGGACGGAGGTCGGC
TCAGCGCGGGCCTCTTTCACTCCGGGCCCCAGCCTTCCCGGTCTCATTTA
CAGTCAGAGGCGCTGAAGCTCAGAAGGTGCATGCGATTCCCCTATTTTTC
TCCCGTAATACTGAGACTCAGGGAAATGGAAATAAACTCTTCTTATTTTT
CAGACGGAACCCGGGGGCTCAGGCAGACCCTAAGTCTTGCCCAAAGCTTC
GCAGTCGGGCAGCAGTGGAGCCAGGATTTGGAAGTAAGAGGACCTG [symbol] GT
GCTTCCCTGCAGTCTCCTGCGCTGCGTCCCACGCCCCGACAACACGCACG
CAACACCTACCCCTGTCTCACCTTGAGCAGATATCGGTCCAGGATCTCGA
GGCCGCAGCTGGAGCAGATGTTCTTGCCTGCAGACGGCACGGAGGAGGCG
GCAGAGGGCGGTGAGCAGACAGATGGCGTGCTGGG [symbol] TACATGGGGAGGC
CTGACCCTCGTCCTTGTCCAGAGCTCCTGCCAGGGCCT [symbol] GCGTCAGACT
GAGCCTGCGGCGGGGGAGAGAAGGAGAGGAG [symbol] CTGATCCGGGCACCCAG
AGTTGGGGCTGCCTTGGAGACAAATCTGGGGCCCCCGAGCACCAATTGAC
AA [symbol] AAATCTCCCCAGGACAATGCGCCGTAGACTGAAGGACAGGGATGGA
AGGGCCTATTGGAGAGGAAGGGACGGCCCCAACTTTTAACACGCGCATTC
TGGGTGCTGGGAGCGGTTAAAGCCAACATTCCCTGCCGGAAAAC [symbol] AGGC
TCAGAGAGGGGCGGCGCCCGCCGGGAGTCCCCCAGAGCGTGAGCAGCAGT
TCGTGGCCGGAAAACCTGGCCAGGTCCCCAGGGCCGGGCCGCTGGAGGAC
TGGGCACCGCAGGCAGGGCCAAACGGACTCACCATGGCGGGCGGCGCGGT
CCCTTCAAGACAGCGGGTGGTCGCTTTGCAGCCGGACCCTGGCTGGGCCA
TCACCTGGGGGAGGGGGGGAGGGAACGCAGGCGGCGGCGGCTGCTGAACT
GGCTCCGCACAGCCTGAGCCCAGCGCCTCCCCGCG

DMP biomarker candidates associated with GABRA2 show significant (aLIS P-value<0.05) association between degree of methylation in target regions and pupillary unrest index measured by infrared pupillometry when DNA samples from subjects with hypersomnolence are analyzed. DMP biomarkers associated with GABRA2 are recited in Table 2.

(CpG DMPs are indicated in BOLD)
SEQ ID NO: 5
CGTTTGACAGCTGCTCTGGAGCCGAGGATCACAATAAGAGAAGGCGCGTG
AGGCTCCGGCAGCAAACACCAGCCAGGCAGGCAGCGGTAATCACAGTGAG
CAAAGTTGAAGACTATAAAAGAGCCAGGCTAACGTGCTGCCGAGCAGGTG
TCCTGGATTTTATTGTAGTTGCAAATATGCTTCTGGTGATAAGTATCGAA
ATTATTTTTTAAGTGCGCTGGGGAGGAGGGAGGTGCGGGAATTGAGCCTC
CTCCGTTTCCAGGGAGCCTCTGAAAGTGGGGTGGGGGGAGATGAGGCATG
CACG

Any combination of DMPs outlined in Tables 1 and 2 may be used to diagnose or give a prognosis of hypersomnolence, idiopathic hypersomnia, or long sleep time in a subject. Any combination of DMPs outlined in Tables 1 and 2 may be used in an assay to quantify methylation. Any combination of DMPs outlined in Tables 1 and 2 may be used in an assay to amplify the DMPs or DMP-associated genes for sequencing to quantify methylation.

In some embodiments, the DMPs of interest are within any one or more of SEQ ID NO:1, SEQ ID NO:2, SEQ ID NO:3, SEQ ID NO:4, SEQ ID NO:5, SEQ ID NO:6, SEQ ID NO:7, SEQ ID NO:10, SEQ ID NO:11, SEQ ID NO:18, SEQ ID NO:19, SEQ ID NO:24, SEQ ID NO:25, SEQ ID NO:34, and SEQ ID NO:35. In some embodiments, DMPs within any one or more of SEQ ID NO:1, SEQ ID NO:2, SEQ ID NO:3, SEQ ID NO:4, SEQ ID NO:5, SEQ ID NO:6, SEQ ID NO:7, SEQ ID NO:10, SEQ ID NO:11, SEQ ID NO:18, SEQ ID NO:19, SEQ ID NO:24, SEQ ID NO:25, SEQ ID NO:34, and SEQ ID NO:35 are assayed to diagnose or give a prognosis of hypersomnolence, idiopathic hypersomnia, or long sleep time in a subject.

In some embodiments, at least 1, at least 2, at least 3, at least 4, at least 5, at least 6, at least 7, at least 8, or all 9 of the DMPs of PAX8 and PAX8-AS1 outlined in Table 1 are assayed to diagnose or give a prognosis of hypersomnolence, idiopathic hypersomnia, or long sleep time in a subject. In some embodiments, at least 1 or both of the DMP-associated genes PAX8 and PAX8-AS1 are assayed to diagnose or give a prognosis of hypersomnolence, idiopathic hypersomnia, or long sleep time.

In some embodiments, at least 1, at least 2, at least 3, at least 4, at least 5, at least 6, at least 7, at least 8, at least 9, at least 10, or all 11 of the DMPs of LHX6 outlined in Table 2 are assayed. In some embodiments, at least 1, at least 2, at least 3, at least 4, at least 5, at least 6, or all 7 of the DMPs of GABRA2 outlined in Table 2 are assayed.

In some embodiments, the biomarkers described herein are used in the production of a biomarker panel for use in assaying DNA methylation. The biomarker panel includes probes or primers specific to the sequences of the DMPs or DMP-associated genes disclosed herein. In some embodiments, the biomarker panel includes probes or primers specific to the sequences of the DMP-associated genes PAX8 and PAX8-AS1. In some embodiments, the biomarker panel includes probes or primers specific to the sequences of the DMP-associated genes LHX6 and GABRA2. In some embodiments, the biomarker panel includes probes and/or primers specific to the DMPs listed in Tables 1 and 2. In some embodiments, the biomarker panel includes probes and/or primers specific to any one or more of SEQ ID NO:1, SEQ ID NO:2, SEQ ID NO:3, SEQ ID NO:4, SEQ ID NO:5, SEQ ID NO:6, SEQ ID NO:7, SEQ ID NO:10, SEQ ID NO:11, SEQ ID NO:18, SEQ ID NO:19, SEQ ID NO:24, SEQ ID NO:25, SEQ ID NO:34, and SEQ ID NO:35.

Primers specific to the DMPs or DMP-associated genes disclosed herein are between about 10 base pairs (bp) and about 40 bp and are complementary to sequences upstream and downstream of the DMP or DMP-associated gene of interest. Generally, a pair of forward and reverse primers that are designed to be complementary to the sequences flanking the DMP or DMP-associated gene are included. The size of the fragment to be amplified by the primer pair can range from less than 50 bp to greater than 10,000 bp. Primers can be designed that are complementary to a sequence less than 50 bp upstream of the DMP or more than 1,000 bp upstream depending on the sequence technology selected and the application of the biomarker panel. Therefore, it is possible to design many permutations of primer sets that are capable of amplifying a given DMP or DMP-associated gene of interest. For example, a given sample containing genomic DNA with a 500 bp DMP, a primer set can be designed to amplify i) the exact target region; or ii) a region encompassing the DMP including upstream and downstream regions. In some embodiments, the size of the fragment to be amplified by the primer pair has a length less than 1000 bp, less than 950 bp, less than 900 bp, less than 850 bp, less than 800 bp, less than 750 bp, less than 700 bp, less than 650 bp, less than 600 bp, less than 550 bp, less than 500 bp, less than 450 bp, less than 400 bp, 350 bp, less than 300 bp, less than 250 bp, less than 200 bp, less than 150 bp, less than 100 bp, less than 75 bp, or less than 500 bp. In some embodiments, the size of the fragment to be amplified by the primer pair has a length of at least 100 bp, at least 125 bp, at least 150 bp, at least 175 bp, or at least 200 bp and less than 400 bp, less than 375 bp, less than 350 bp, less than 325 bp, or less than 300 bp.

Probes specific to the DMPs or DMP-associated genes disclosed herein are between about 10 bp and about 40 bp and are commentary to sequences including or adjacent to the DMP or DMP-associated gene of interest. In some embodiments, the probe is complementary to the DMP of interest.

The disclosure includes a number of preferred primers and probes for amplification, selection, and identification of specific DMPs or DMP-associated genes.

However, a skilled artisan will appreciate that the DMPs and DMP-associated genes disclosed can be amplified, selected, and identified by primers and probes other than those specific disclosed, which have been presented for purposes of illustration. We envision that the biomarker panel is compatible with a number of amplification and sequencing schemes and the scope of the claims should not be limited to the description of the embodiments contained herein.

Probes or primers for use in the biomarker panels described herein may be fused to a tag or label. Suitable tags and labels are known in the art, including but not limited to fluorescent labels (e.g., GFP, RFP, etc.), biotin, and combinations thereof. In some embodiments, the probe or primer is biotinylated and the biotinylated probe or primer bound sequence can be purified or captured with a streptavidin bound substrate.

In some embodiments, the primers or probes are covalently or non-covalently linked to a substrate. Suitable substrates for the biomarker panel include a bead, a plate, a microfluidic devise, a cuvette, a chip, a multiwell plate (e.g. 6-, 12-, 24-, 48-, 96-, 384-, or 1536-well plates).

Primers specific to the DMPs or DMP-associated genes disclosed herein can be obtained using a number of methods known in the art. In one example, the abundance of 5mC and/or 5hmC in regions of interest can be interrogated (see below for an exemplary method). Once regions of interest are identified, genomic sequences corresponding to each locus can be obtained from a reference genome and imported to a methyl primer design software package, such as the Thermo Fisher Scientific Methyl Primer Express v1.0 software, to generate high quality PCR primers pairs for methylation mapping. A second pair of primers can be designed that contains the initial primer sequence flanked by a sequencing adapter sequence (e.g., Illumina or others). Primers can be ordered through any preferred provider.

An exemplary method for interrogating the abundance of 5mC and/or 5hmC in regions of interest can be performed with the Methyl Primer Express™ Software v1.0

(Thermo Fisher Scientific). Methyl Primer Express™ can be downloaded from the

Thermo Fisher Scientific Applied Biosystems web site (www.thermofisher.com/us/en/home.html). The target sequence can be input into the box labeled “Insert Nucleotide Sequence.” (To increase the likelihood of finding primers that will create an amplicon of ˜200 base pairs (bp) or larger, input a minimum sequence of ˜500 base pairs. The longer the input sequence, the better chance of finding suitable primers to target the region of interest.) “Design Primers” can then be clicked. “Select Target Sequence” can then be chosen to select the specific region of interest within the input DNA sequence. (As above, the longer the target sequence selected, the more likely it is to find suitable primers.) Following selection of primers, “Next” can be pressed, and a new window will open. If finding CpG islands within the target sequence is of interest, “Find CpG Islands” can be clicked. (Check the current default parameters that define a CpG island. If the input sequence is only 500 base pairs, consider shortening the defined minimum length of the island, as the length of a CpG island will later serve to help in choosing where to design the primers. It is possible that these analyses may not yield any CpG islands. If this is the case, simply move on to the next step.) In the bottom right corner of the new window, bisulfite sequencing primer design can be selected by clicking “Design BSP Primers.” (BSP=Bisulfite Sequencing Primers. MSP=Methylation-Specific Primers.) Parameters used to design the BSPs can be reviewed by clicking “Next.” (It is preferred to design primers that will generate an amplicon length of 200 to 300 base pairs. Amplicons<200 base pairs will result in sequencing the adaptors, meaning it will not maximize the data from the final sequencing run. Primers are preferably between 20 to 30 base pairs in length. Annealing temperature of the PCR should have a small range (e.g., 58° to 60° C.).) It is preferred to initially weight performance towards the Low Speed/High Accuracy setting (between 8 to 10). If no appropriate primers are found, the scale can be moved towards the Hi Speed/Low Accuracy; however, this is not preferred. The designed primers can be purchased through any of a number of standard primer providers. Exemplary parameters include 25 nmole DNA oligos, standard desalting, and RNase free water (unless shipped desiccated). Two to three primer pairs per target region can be designed and purchased for simultaneous testing.

Exemplary non-modified and bisulfite-modified regions of interest for PAX8/PAX8-AS1, LHX6, and GABRA2 comprising the DMPs disclosed herein and exemplary primers for amplifying the DMPs are provided below. All positions below refer to the UCSC hg19 human reference genome assembly.

PAX8/PAX8-AS1
PAX8/PAX8-AS1 Region of Interest 1:
chr2: 113,992,930-113,993,314 5′pad = 250
3′pad = 250
Non-Modified PAX8/PAX8-AS1 Region of
Interest 1 Sequence:
(SEQ ID NO: 6)
AGGCCAAGTTGCTACGTTACAGCACCCGGCTCCCGGCCTTAGGGTTCCTG
CTTTTCCATGGGGTCTTGGGGTGGTGCATCCCCGGCACCCCTACAGCATC
CGCCCCTTCCGAGCATGTCTTCCCCGTCACAGAGAACTTCATGTTGGCGC
CTCCAAAAGTTGCCGGAGGAATTAGGGAACAGGGTGGGGGTGGATGAGAC
TGAGGCCAGAGAGGGGGCTGGCGGTCTGCCCTGAGGACCCCCGTCCCACC
CGCCGCCATAGCTGCATGGCCCCGGGACCTCCCTGTCGTACCTGAGAGGA
GGGCCTGGCCCGTGAACTGCCCGTACACGGAGGCAGCATGGGGAAAGGCA
TTGAAGGGCGGGACCCCGGAGCCGACTTGCTGCAGATCCAAAAAGGCGGA
GCTAGATAAAGAGGAAGGGGTGGAGCTAGAACTGGACACCTCGGGGGTTT
CCTGCTTTATGGCGAAGGGTGAGTGAGGATCTGCCGGAGGGAGGGAGACA
ACAAGGAGAGAGGGGTGTGAGATGGCGGGGAGGGAACACGCACAAGCCAA
GCCGTGGGATGTGGAGGGTGCGGGCGGGGCGCGGGGCAGGCTCAGCTGCC
CTCAGAGTCTGGGCTGGGGAGAGCCGGGGCCCACGAGGCGTGGCAAGGCG
GGGAGAGAGAAGCTAGACCTCCCTGCTGCGCTTCTGCAGCGAAAATGGAG
AGACCCGGAAGGCGTGTGTGCGCCCGCCTCTCCCACAGGAGGGAGCGCGG
AGACCCGGGAGCGGCCTAGGACCGGAGGCGCGACCCCTCGGCCCACCTTG
AGGCCCGGCCTAGGACCGGAGGCGCGACCCCTGGGCCCACCTTGAGGCCC
GGCCTAGGACTGGAGGCGCGACCCCTCGGCCCACC
Bisulfite-Modified PAX8/PAX8-AS1
Region of Interest 1 Sequence:
(SEQ ID NO: 7)
AGGTTAAGTTGTTACGTTATAGTATTCGGTTTTCGGTTTTAGGGTTTTTG
TTTTTTTATGGGGTTTTGGGGTGGTGTATTTTCGGTATTTTTATAGTATT
CGTTTTTTTCGAGTATGTTTTTTTCGTTATAGAGAATTTTATGTTGGCGT
TTTTAAAAGTTGTCGGAGGAATTAGGGAATAGGGTGGGGGTGGATGAGAT
TGAGGTTAGAGAGGGGGTTGGCGGTTTGTTTTGAGGATTTTCGTTTTATT
CGTCGTTATAGTTGTATGGTTTCGGGATTTTTTTGTCGTATTTGAGAGGA
GGGTTTGGTTCGTGAATTGTTCGTATACGGAGGTAGTATGGGGAAAGGTA
TTGAAGGGCGGGATTTCGGAGTCGATTTGTTGTAGATTTAAAAAGGCGGA
GTTAGATAAAGAGGAAGGGGTGGAGTTAGAATTGGATATTTCGGGGGTTT
TTTGTTTTATGGCGAAGGGTGAGTGAGGATTTGTCGGAGGGAGGGAGATA
ATAAGGAGAGAGGGGTGTGAGATGGCGGGGAGGGAATACGTATAAGTTAA
GTCGTGGGATGTGGAGGGTGCGGGCGGGGCGCGGGGTAGGTTTAGTTGTT
TTTAGAGTTTGGGTTGGGGAGAGTCGGGGTTTACGAGGCGTGGTAAGGCG
GGGAGAGAGAAGTTAGATTTTTTTGTTGCGTTTTTGTAGCGAAAATGGAG
AGATTCGGAAGGCGTGTGTGCGTTCGTTTTTTTTATAGGAGGGAGCGCGG
AGATTCGGGAGCGGTTTAGGATCGGAGGCGCGATTTTTCGGTTTATTTTG
AGGTTCGGTTTAGGATCGGAGGCGCGATTTTTGGGTTTATTTTGAGGTTC
GGTTTAGGATTGGAGGCGCGATTTTTCGGTTTATT
Exemplary Primer Sequences for
PAX8/PAX8-AS1 Region of Interest 1
Amplification:
Forward primer:
(SEQ ID NO: 8)
GAGGTTAGAGAGGGGGTTGG
Reverse primer:
(SEQ ID NO: 9)
CGCTACAAAAACGCAACAAA
PAX8/PAX8-AS1 Region of Interest 2:
chr2: 113993785-113995706 5′pad = 250
3′pad = 250
Non-Modified PAX8/PAX8-AS1
Region of Interest 2 Sequence:
(SEQ ID NO: 10)
CCTTACGGGAGCCGCCTAGGGCCGGAGGCGCGACCCCTCGGCCCAGCTTG
AGGCCCGGCCTAGGACCGGAGGCGCGACCCCTCGGCCCACCTTGAGGCCC
GGCCTAGGACTGGAGGCGCGACCCCTCGGCCCACCTTGAGGCCCGGCCTA
GGACCGGAGGCGCGACCCCTGGGCCCACCTTGAGGCCCGGCCTAGGACTG
GAGGCGCGACCCCTCGGCCCACCTTGAGGCCCGGCCTAGGACCGGAGGCG
CGACCCCTGGGCCCACCTGGCGGCCCGGCCGGCACAGCCCGCCTCTCCTC
TCCAGGCCAGGGCCCCACACCTTCCGCCTGACAGCCAGCCAAGCTCTTCA
GTCCCCCGCCCTCCACCTGCCAGGGAGGCTCCGGGCGTTGTACCTGCCAC
CACGGGGTAGGTCTGGTGAGTCGAGAGGTTGCGCCCCAGTGGCGTGTTGG
AAGGGGTCAGGGTGGCCTTCCCGTCGTCCAGGGTGCTGTTGAGCAAGGGC
AGCGGGTAGAGGCCCTGGGGAGCAAAGAGAAGTCAGCGCACAGAGACGAT
CCAACAAGCCGACCTTCCTGCAGACCCTGAGCCTGTGTCTTAGGACTTGG
CAGCCCTCATCTCCCCAGGAGAGGTCCTCATCGCCCCCTTCTTCCTTTAC
GTTCTCAACTCCACTCGGCACGTTTCGTTCATGCTCATTGTCCTCCCGCA
TCCCTGGAATTCAAGGCCAGCTGGTAGCATGGGTGCCCAGACGTGGGATA
GAGAGTCTCAGCCAGAGGACCACACCCTGGTTGGATGGCTGGTCGGCTTC
CTGGTTTCACCACACTGTTGGGACCCACAGAATGCAGGACGGTGCCAAGG
ACTGGAGCAGTCTCCAACCGGACTGTTCAGGGTCCTTGCATCTGTTAATT
TATTCTTCCCCAGATAGGAAACCCAGGCACAAAGGTTTAAGAAACTTGTC
CCACTTCCAGATGGGCCAAAGATAGGATTCAGATTCGAGGGATCAGAATG
TGTCTGCTCTCAACCACTGCCCTAAACTGGGTGACATAAGGACCACTTCT
TACATTTGCAAAGAGCTTATATCAGAGCTTTGCCTCTGTTGTCCTAGTAG
AGTCCCCCAGCAGCCCAGTGACATGGGTGAGTAATATTATGTCTGTGTTT
TATATGTGGAGCCTGGAACTCAGGTAAATGGCCCAATTTTGTATAGTGAA
ACAGACTAAACCCGGGATTTTTCGGCTGTGGACCCAGGTGCTTTACCCAC
CACGTGGATTTCTTCTTTTCTCTCTCCACATCCAAACAAATAGAAGTGTG
CAAGTTCGGCTTGAAAATGGTATTGAGAGTTGTTTTTGTTGGAAACACTC
TTTGTCAAGTACCTGGGGGTCTTCATCAGTAAGACCTCATTTTAGATGCC
TCCTGGCCTCCCCTTCCCAGGAGCACAGCTATGACCTTAGGTACTCCTTC
CGAAAAGAACTTGTTTAACTAAAGGTAAGTGTACCTCATCCTCACCATGG
CCTCCTTCCACTGGGGAAGCAGATAGCGCAGAAAAAAGAACACACCCATT
CCCCACATACCTTCACACTCGTCACATACCTGCTACGTGAGATGTGCAAA
GCTGAATTCAGGGAATGCTCAGTAGTTACATAACAGTGCCACTAAAGGCA
ATTGTTTTCAGTGATTTCCATCGAGCTGGGTTCTGCAAAGATCCACAGCA
CTTTCCGGTTGCATGCTGGGCACTTTTGGAAGCTGCAGTCAATTCTGGAG
GCCACCAGGGCACCATTAGCACATAGCAGCAATTATTGACTAAATGGTGC
TCTGGTTCCATGCCTTCCAAGGGGGCCCACTTAGAGGCAGGGTGGAGTTG
CTTAGGGCCTTTTTTTTTTTTTTTTTTTGTAGACGGAGTTTTGCTCTTGT
TGCCCAAGCTGGAGTGCAATGG
Bisulfite-Modified PAX8/PAX8-AS1
Region of Interest 2 Sequence:
(SEQ ID NO: 11)
TTTTACGGGAGTCGTTTAGGGTCGGAGGCGCGATTTTTCGGTTTAGTTTG
AGGTTCGGTTTAGGATCGGAGGCGCGATTTTTCGGTTTATTTTGAGGTTC
GGTTTAGGATTGGAGGCGCGATTTTTCGGTTTATTTTGAGGTTCGGTTTA
GGATCGGAGGCGCGATTTTTGGGTTTATTTTGAGGTTCGGTTTAGGATTG
GAGGCGCGATTTTTCGGTTTATTTTGAGGTTCGGTTTAGGATCGGAGGCG
CGATTTTTGGGTTTATTTGGCGGTTCGGTCGGTATAGTTCGTTTTTTTTT
TTTAGGTTAGGGTTTTATATTTTTCGTTTGATAGTTAGTTAAGTTTTTTA
GTTTTTCGTTTTTTATTTGTTAGGGAGGTTTCGGGCGTTGTATTTGTTAT
TACGGGGTAGGTTTGGTGAGTCGAGAGGTTGCGTTTTAGTGGCGTGTTGG
AAGGGGTTAGGGTGGTTTTTTCGTCGTTTAGGGTGTTGTTGAGTAAGGGT
AGCGGGTAGAGGTTTTGGGGAGTAAAGAGAAGTTAGCGTATAGAGACGAT
TTAATAAGTCGATTTTTTTGTAGATTTTGAGTTTGTGTTTTAGGATTTGG
TAGTTTTTATTTTTTTAGGAGAGGTTTTTATCGTTTTTTTTTTTTTTTAC
GTTTTTAATTTTATTCGGTACGTTTCGTTTATGTTTATTGTTTTTTCGTA
TTTTTGGAATTTAAGGTTAGTTGGTAGTATGGGTGTTTAGACGTGGGATA
GAGAGTTTTAGTTAGAGGATTATA [symbol] [symbol] [symbol] CGGTTTT
TTGGTTTTATTATATTGTTGGGATTTATAGAATGTAGGACGGTGTTAAGG
ATTGGAGTAGTTTTTAATCGGATTGTTTAGGGTTTTTGTATTTGTTAATT
TATTTTTTTTTAGATAGGAAATTTAGGTATAAAGGTTTAAGAAATTTGTT
TTATTTTTAGATGGGTTAAAGATAGGATTTAGATTCGAGGGATTAGAATG
TGTTTGTTTTTAATTATTGTTTTAAATTGGGTGATATAAGGATTATTTTT
TATATTTGTAAAGAGTTTATATTAGAGTTTTGTTTTTGTTGTTTTAGTAG
AGTTTTTTAGTAGTTTAGTGATATGGGTGAGTAATATTATGTTTGTGTTT
TATATGTGGAGTTTGGAATTTAGGTAAATGGTTTAATTTTGTATAGTGAA
ATAGATTAA [symbol] [symbol] [symbol] GGATTTAGGTGTTTTATTTAT
TACGTGGATTTTTTTTTTTTTTTTTTTATATTTAAATAAATAGAAGTGTG
TAAGTTCGGTTTGAAAATGGTATTGAGAGTTGTTTTTGTTGGAAATATTT
TTTGTTAAGTATTTGGGGGTTTTTATTAGTAAGATTTTATTTTAGATGTT
TTTTGGTTTTTTTTTTTTAGGAGTATAGTTATGATTTTAGGTATTTTTTT
CGAAAAGAATTTGTTTAATTAAAGGTAAGTGTATTTTATTTTTATTATGG
TTTTTTTTTATT [symbol] [symbol] AAAGAATATATTTATT
TTTTATATATTTTTATATTCGTTATATATTTGTTACGTGAGATGTGTAAA
GTTGAATTTAGGGAATGTTTAGTAGTTATATAATAGTGTTATTAAAGGTA
ATTGTTTTTAGTGATTTTTATCGAGTTGGGTTTTGTAAAGATTTATAGTA
TTTTTCGGTTGTATGTTGGGTATTTTTGGAAGTTGTAGTTAATTTTGGAG
GTTATTAGGGTATTATTAGTATATAGTAGTAATTATTGATTAAATGGTGT
TTTGGTTTTATGTTTTTTAAGGGGGTTTATTTAGAGGTAGGGTGGAGTTG
TTTAGGGTTTTTTTTTTTTTTTTTTTTTGTAGACGGAGTTTTGTTTTTGT
TGTTTAAGTTGGAGTGTAATGG
Exemplary Primer Sequences for
Amplification of PAX8/PAX8-AS1
Region of Interest 1
(Chr2: 113994035-113995456):
Forward primer1:
(SEQ ID NO: 12)
TACGGGAGTCGTTTAGGG
Reverse primer1:
(SEQ ID NO: 13)
CTCAACAACACCCTAAAC
Forward primer2:
(SEQ ID NO: 14)
ATTTTGGTTGGATGGTTGGT
Reverse primer2:
(SEQ ID NO: 15)
ACAACCGAAAAATCCCGAAT
Forward primer3:
(SEQ ID NO: 16)
TGGGGAAGTAGATAGCGTAGAAA
Reverse primer3:
(SEQ ID NO: 17)
CCCTAAACAACTCCACCCTACC
LHX6
LHX6 Region of Interest 1:
chr9: 124,989,052-124,989,551
5′pad = 250 3′pad = 250
Non-Modified LHX6 Region of
Interest 1 Sequence:
(SEQ ID NO: 18)
CGGCTCAGCGCGGGCCTCTTTCACTCCGGGCCCCAGCCTTCCCGGTCTCA
TTTACAGTCAGAGGCGCTGAAGCTCAGAAGGTGCATGCGATTCCCCTATT
TTTCTCCCGTAATACTGAGACTCAGGGAAATGGAAATAAACTCTTCTTAT
TTTTCAGACGGAACCCGGGGGCTCAGGCAGACCCTAAGTCTTGCCCAAAG
CTTCGCAGTCGGGCAGCAGTGGAGCCAGGATTTGGAAGTAAGAGGACCTG
CGGTGCTTCCCTGCAGTCTCCTGCGCTGCGTCCCACGCCCCGACAACACG
CACGCAACACCTACCCCTGTCTCACCTTGAGCAGATATCGGTCCAGGATC
TCGAGGCCGCAGCTGGAGCAGATGTTCTTGCCTGCAGACGGCACGGAGGA
GGCGGCAGAGGGCGGTGAGCAGACAGATGGCGTGCTGGGCGTACATGGGG
AGGCCTGACCCTCGTCCTTGTCCAGAGCTCCTGCCAGGGCCTCGGCGTCA
GACTGAGCCTGCGGCGGGGGAGAGAAGGAGAGGAGCGCTGATCCGGGCAC
CCAGAGTTGGGGCTGCCTTGGAGACAAATCTGGGGCCCCCGAGCACCAAT
TGACAACGAAATCTCCCCAGGACAATGCGCCGTAGACTGAAGGACAGGGA
TGGAAGGGCCTATTGGAGAGGAAGGGACGGCCCCAACTTTTAACACGCGC
ATTCTGGGTGCTGGGAGCGGTTAAAGCCAACATTCCCTGCCGGAAAACCG
AGGCTCAGAGAGGGGCGGCGCCCGCCGGGAGTCCCCCAGAGCGTGAGCAG
CAGTTCGTGGCCGGAAAACCTGGCCAGGTCCCCAGGGCCGGGCCGCTGGA
GGACTGGGCACCGCAGGCAGGGCCAAACGGACTCACCATGGCGGGCGGCG
CGGTCCCTTCAAGACAGCGGGTGGTCGCTTTGCAGCCGGACCCTGGCTGG
GCCATCACCTGGGGGAGGGGGGGAGGGAACGCAGGCGGCGGCGGCTGCTG
Bisulfite-Modified LHX6 Region of
Interest 1 Sequence:
(SEQ ID NO: 19)
CGGTTTAGCGCGGGTTTTTTTTATTTCGGGTTTTAGTTTTTTCGGTTTTA
TTTATAGTTAGAGGCGTTGAAGTTTAGAAGGTGTATGCGATTTTTTTATT
TTTTTTTCGTAATATTGAGATTTAGGGAAATGGAAATAAATTTTTTTTAT
TTTTTAGACGGAATTCGGGGGTTTAGGTAGATTTTAAGTTTTGTTTAAAG
TTTCGTAGTCGGGTAGTAGTGGAGTTAGGATTTGGAAGTAAGAGGATTTG
CGGTGTTTTTTTGTAGTTTTTTGCGTTGCGTTTTACGTTTCGATAATACG
TACGTAATATTTATTTTTGTTTTATTTTGAGTAGATATCGGTTTAGGATT
TCGAGGTCGTAGTTGGAGTAGATGTTTTTGTTTGTAGACGGTACGGAGGA
GGCGGTAGAGGGCGGTGAGTAGATAGATGGCGTGTTGGG [symbol] [symbol] [symbol]
TTTTCGTTTTTGTTTAGAGTTTTTGTTAGGGTTTCGGCGT
TAGATTGAGTTTGCGGCGGGGGAGAGAAGGAGAGGAGCGTTGATTCGGGT
ATTTAGAGTTGGGGTTGTTTTGGAGATAAATTTGGGGTTTTCGAGTATTA
ATTGATAACGAAATTTTTTTAGGATAATGCGTCGTAGATTGAAGGATAGG
GATGGAAGGGTTTATTGGAGAGGAAGGGACGGTTTTAATTTTTAATACGC
GTATTTTGGGTGTTGGGAGCGGTTAAAGTTAATATTTTTTGTCGGAAAAT
CGAGGTTTAGAGAGGGGCGGCGTTCGTCGGGAGTTTTTTAGAGCGTGAGT
AGTAGTTCGTGGTCGGAAAATTTGGTTAGGTTTTTAGGGTCGGGTC [symbol] [symbol]
TAGGGTTAAACGGATTTATTATGGCGG
GCGGCGCGGTTTTTTTAAGATAGCGGGTGGTCGTTTTGTAGTCGGATTTT
GGTTGGGTTATTATTTGGGGGAGGGGGGGAGGGAACGTAGGCGGCGGCGG
TTGTTG
Exemplary Primer Sequences for
Amplification of LHX6 Region of
Interest 1:
Forward primer A:
(SEQ ID NO: 20)
GGAATTCGGGGGTTTAGGTA
Reverse primer A:
(SEQ ID NO: 21)
ACGCTCCTCTCCTTCTCTCC
Forward primer B:
(SEQ ID NO: 22)
GCGTATATGGGGAGGTTTGA
Reverse primer B:
(SEQ ID NO: 23)
CCTACGATACCCAATCCTCCAAC
LHX6 Region of Interest 2:
chr9: 124985156-124990089
5′pad = 250 3′pad = 250
Non-Modified LHX6 Region of
Interest 2 Sequence:
(SEQ ID NO: 24)
TTGGGAGCTCTTGGCCTCTCTTTGGGCAGAGTGCTCCAGTCCACATCCTA
CCAGACCCTTGACCCCGGGCTTCAGGAAACAGAAGCCTAGAGAACCTCAG
CATGTGACCCCTCCCTGGGCCCCAGCTCCTTAGAGGAAAATTCTGCCTCC
CACGCCACCATTCACACTCTCTCCAGGAAGAACTTCCTCATAGCAGCCAC
CCAGAGCTTGGGCTGCAGTGCAGTGCAGACAATAGTCCTTGCCTGGCACT
CGATCGGCTTTTTCTTTCCCTTTTTGTGGGTTTTATTATTTTTGTTACAG
GTGGGGCTGCCAGGGATGGGTTATGCCTGCAGGGGGAGAAATGATCAGAA
GAGCCACAATTCTACCCTCAACCCAGGGAATGTGAGCTTTTACTGCCTCC
ATTTTCTTAACCAGAAATCTGGTTTAGTTTTGCTTTCAGGGCTCTTGTCA
TTGGAAAAAGATTAGTGCTCAGAACCTGCAGCCCAGAAATGGCCCAGTCC
CATCCCTCCAGGCAGGCTCTGCTCCTCAAGCTGCGTGGGGAAATGCCATC
ATGGGCTTGTGCCCAGCATCCACCCTGCCAATGATGACAACAGTGATAAT
CGAGGCATTCATACCAACCACCATTTAGGGAGCCTTTATAGAGACTTCCC
CCCAACAGTGGCCTCTTTGGCAGCTCTGGGAGACAGCCTTGGAAAGGGCG
AACTAAGGGCCGAGAGAAGCAGCAACTTGCGAAGGTCACTACTGGCCAGA
CTGGGCCCAGTCTCCTGCCTCTTCCCACAGTGGCTGCACACCCTACTCTC
TACCCCATTGCAGTAGGAAGAGAACTCCAGAATAAGGGGAGGCTCTGAAC
AGTGGAGTCAGGATCCGTGTTTTGACTAGTTTCAAGGTCTAGTAAAGCAC
AGTATCCAAGGAGGCTCAGTCCAGCATGGGCATGGGCTAGGGCTGGAGAA
GTAGAAAAGAAATTTGGGCAAGTAGTGAATAAGACTGCCAAATCTATTGT
TAGGAGAGCCAGGAGAGGTGTCCTAGTGGTGCCTTAGTCTATGGGAGAGA
CCCAGACACCATTCTGCTGGGTTCCCAAGCTGGCACCTTAAATTTAGTAG
CTGATGAATCCAAGGAGCCCCCAAGCATCATCTGGCATGTTTTTTTGTAT
GTTTGTTTGTTTGTTTTTTGAGACAGAGTCTCTCTCTGTTACCCAGGCTG
GAGTGTAGTGACGTGATCTTGGCTCACCACAATGTCCGCCTCCCAGGTTC
AAGTGATTCTCTTGCCTCAGCCTCCTGAGTAGCTGGGACTACAGGCACGT
GCCACCACACCTGGCTAAGTTTTGTATTTTTAGTAGAGATGGGTTTCACC
ATGTTGGCCAGGATGGTCTCGATCTCCTGACCTTGTGATCTACCAGCCTT
GGCCTCCCAAAGTGCTGCGATTATAGGTGTGAGCCACCACACCCGGCCCA
TCTGGCATGTTTTATACCAACTGGAGTCAGGCCTAGAGCAGGGAGGTGGC
TAGACAGTGACTGTAACCTCCATTTCCTGTCCTGGCCAAACCAATACTCA
GATGGGACAGTGAGAGGAGACATCACACTCTGGTTACACACCATTAACCC
CTGTCTGACTACACACACACACACACACAGCCGTCCAGATTCACAAATAC
GTTCTTATCACCCCCTTCATCCCTCAGTGCCTGGGTGGAGGCCCTTAAGC
AGGAATGCCCCCTCAGAGGAGAGGGTGGGAGTGGAATGATGGGATTTCAG
AATCCCCTCACCTCACCCTCTCACCTCCAGATCCTAGTCCAGATGTAATC
CTGTCCTCACCACTTTGCGTGCACGTGCACGCACACACACATGCACACAC
ACAGTCTCAACATTCACATACAGACACCCACTCTTGACACCCACACATGA
ACTAAAAGACAAACGTTGGTCCATAAAGGAACACACATGCTATCTACATA
CCCCCAACCCTGCCCAGTGATGCTTTCCCCAGCACTACTGCACAGATCCC
AGACAATACAGACACACAGGCACTCCTTGCACACTGAGTCCTAGGCCACT
TGGTTGAGCTGCCCCCACCCCACCCCAGCTGCACACTCTTCTACATCCAT
CCCATAAAGGAAGAGTCCGTCTCTTTAACCATGCTTCTCATGGTTTTCAG
AGTACCCTTGCCCCAAATGCTGCCAACCTCCCCCTAGACTCAAGATCTTC
TCTTTCCAGATCGCTGGAGGCTCAATGCCAGTGCACAGGAGGAGATCGCC
TTCCACCATATTCCCTGACTCCCAGAGGCTCTTTCTAGGGCAGGGGTCTG
GCCAGGAAGACTGAAGCCATGACGTCTTCCTGTGCAAGGATAGGAAAGAT
GTCTGTGGGTTGGAGGGTAGTTATAGGGAAGCAGCTTTCTCCACTTTTTG
AGAAAGCACTTGGGAACAAGCATCCCTCTCAGGAAGTGATTCTCCACCAT
TCTCCTCTGATCCTATAGGCCTGGTGGTCCAAGCCTCAGGCCAGTTGATG
CCTCCCACTGGGATCAGCAGACAAGGCCATGACCCTGCAGTGACCTCGAC
CCCATAATCAGGTCAGGTCCGGGCTTCATGGCTAGAAGCGAGGCCCAGGC
GAGTGGCCAAGACGCGGCAGGACAGGAGGATGCGCCTGGCTGCTCTCGGC
TGCTGGACGCGGCCTGCAGTAGGGAGAGCGCAGCCCGGGGTAATCCCACT
CAGGGCTCCGGGTGCCCAGTTCTCCCACTTCCCATCCTGGCGGCCTAAAG
GCAAGGCCTAGACCCTGGAGAGGCCAAAGTCGGGCCAAAACAACAGCGAA
GCTGGGACTGGAACAGGCGCTGGGGGCCTGCGCATGACCCCAGGCTCAGA
ATGCGACACTCCAGTGACGCCGGGGAATGAGTTGAGATGGAGCCGGAGAC
AGAGGTTGGGGTTCAGGACGAGGCGCTTGGGTATGGGATCGGATCTGAGA
CGGTTGCGGCGCTGGTGCTTTGGGCTGGACCAGACTCGAACCCAGAGCGG
AGCGAGGGTCAGAGCCAGGGCAAGAGTCCGAAGCCCAGCTCAGCGTCCAG
CCCGCGAGAGACCCCACCATGAATGACGTCCGAGTAGGGTTGGAACTCGA
AGCCCGAACGCACTGGAAATGCAGCCGGACGACGGCTGGGATCCGAGTGG
GTCCGGGCCAGAAATGGGGACCTCAGAGCTCCACCGAGGCGCTCGAGGTC
AGAACTGAAGCCTGAGACTTAGGCAGGACCCGAGACAGAGCCAGAGACGA
TACCGAAACCCAATGGACCGCGAGGACCGAAGGCAGATCCGGGGCGCAAA
CCTGTGCAGGCACTGGCGCGCTGCCTGGAGCTCTGGGTTCGCGCCGCTGA
GCGCCGGCAGGTTGGACCAGCGCTGCGCGCCGGAAGTGCACTCGGGACGC
CGGGGTTCTGGAGGAGAGACCACACGCCGCAAAAGTGGCCTCCGAATGCG
CCCGGGGCCTGCCCTCGGCGACACTGCTGGCTCGGCGGCCGCAGCCTACC
TGAAGTAGTCCATCTTGCAGAAGATCTCCTTGTTCTTGATGTAGCAGCTG
TTCTGCTGCCTCAGCGACGTGCGACACACGGAGCACTCGAGGCACCGCAC
GTGCCAGATGAGGTTGTTGACCTGGGGACGGGGCGGGGACGGAGGTCGGC
TCAGCGCGGGCCTCTTTCACTCCGGGCCCCAGCCTTCCCGGTCTCATTTA
CAGTCAGAGGCGCTGAAGCTCAGAAGGTGCATGCGATTCCCCTATTTTTC
TCCCGTAATACTGAGACTCAGGGAAATGGAAATAAACTCTTCTTATTTTT
CAGACGGAACCCGGGGGCTCAGGCAGACCCTAAGTCTTGCCCAAAGCTTC
GCAGTCGGGCAGCAGTGGAGCCAGGATTTGGAAGTAAGAGGACCTGCGGT
GCTTCCCTGCAGTCTCCTGCGCTGCGTCCCACGCCCCGACAACACGCACG
CAACACCTACCCCTGTCTCACCTTGAGCAGATATCGGTCCAGGATCTCGA
GGCCGCAGCTGGAGCAGATGTTCTTGCCTGCAGACGGCACGGAGGAGGCG
GCAGAGGGCGGTGAGCAGACAGATGGCGTGCTGGGCGTACATGGGGAGGC
CTGACCCTCGTCCTTGTCCAGAGCTCCTGCCAGGGCCTCGGCGTCAGACT
GAGCCTGCGGCGGGGGAGAGAAGGAGAGGAGCGCTGATCCGGGCACCCAG
AGTTGGGGCTGCCTTGGAGACAAATCTGGGGCCCCCGAGCACCAATTGAC
AACGAAATCTCCCCAGGACAATGCGCCGTAGACTGAAGGACAGGGATGGA
AGGGCCTATTGGAGAGGAAGGGACGGCCCCAACTTTTAACACGCGCATTC
TGGGTGCTGGGAGCGGTTAAAGCCAACATTCCCTGCCGGAAAACCGAGGC
TCAGAGAGGGGCGGCGCCCGCCGGGAGTCCCCCAGAGCGTGAGCAGCAGT
TCGTGGCCGGAAAACCTGGCCAGGTCCCCAGGGCCGGGCCGCTGGAGGAC
TGGGCACCGCAGGCAGGGCCAAACGGACTCACCATGGCGGGCGGCGCGGT
CCCTTCAAGACAGCGGGTGGTCGCTTTGCAGCCGGACCCTGGCTGGGCCA
TCACCTGGGGGAGGGGGGGAGGGAACGCAGGCGGCGGCGGCTGCTGAACT
GGCTCCGCACAGCCTGAGCCCAGCGCCTCCCCGCGCCTGTTATATAAACC
GGCGCCGAACAATGAGTCCTAACTTTGTAGTGGGCATTTAAAGCCCTTCC
CCACAAAAGCGGTGTCTCTCTAATGAAGCAATTTGAATTTGGATTGGATT
TTTTCCCTCTCTCTCCCCCTCTCCCTGCACTTAACCCGTGGCTCTTGAAG
TAATCGCTTAGTTCCCTTGCAATCCAAGCCTCTGAGGGGGGAAGAAAAAC
ACGCGCACACACACACAAACTACCTGCAATTAGA
Bisulfite-Modified LHX6 Region of
Interest 2 Sequence:
(SEQ ID NO: 25)
TTGGGAGTTTTTGGTTTTTTTTTGGGTAGAGTGTTTTAGTTTATATTTTA
TTAGATTTTTGATTTCGGGTTTTAGGAAATAGAAGTTTAGAGAATTTTAG
TATGTGATTTTTTTTTGGGTTTTAGTTTTTTAGAGGAAAATTTTGTTTTT
TACGTTATTATTTATATTTTTTTTAGGAAGAATTTTTTTATAGTAGTTAT
TTAGAGTTTGGGTTGTAGTGTAGTGTAGATAATAGTTTTTGTTTGGTATT
CGATCGGTTTTTTTTTTTTTTTTTTGTGGGTTTTATTATTTTTGTTATAG
GTGGGGTTGTTAGGGATGGGTTATGTTTGTAGGGGGAGAAATGATTAGAA
GAGTTATAATTTTATTTTTAATTTAGGGAATGTGAGTTTTTATTGTTTTT
ATTTTTTTAATTAGAAATTTGGTTTAGTTTTGTTTTTAGGGTTTTTGTTA
TTGGAAAAAGATTAGTGTTTAGAATTTGTAGTTTAGAAATGGTTTAGTTT
TATTTTTTTAGGTAGGTTTTGTTTTTTAAGTTGCGTGGGGAAATGTTATT
ATGGGTTTGTGTTTAGTATTTATTTTGTTAATGATGATAATAGTGATAAT
CGAGGTATTTATATTAATTATTATTTAGGGAGTTTTTATAGAGATTTTTT
TTTAATAGTGGTTTTTTTGGTAGTTTTGGGAGATAGTTTTGGAAAGGGCG
AATTAAGGGTCGAGAGAAGTAGTAATTTGCGAAGGTTATTATTGGTTAGA
TTGGGTTTAGTTTTTTGTTTTTTTTTATAGTGGTTGTATATTTTATTTTT
TATTTTATTGTAGTAGGAAGAGAATTTTAGAATAAGGGGAGGTTTTGAAT
AGTGGAGTTAGGATTCGTGTTTTGATTAGTTTTAAGGTTTAGTAAAGTAT
AGTATTTAAGGAGGTTTAGTTTAGTATGGGTATGGGTTAGGGTTGGAGAA
GTAGAAAAGAAATTTGGGTAAGTAGTGAATAAGATTGTTAAATTTATTGT
TAGGAGAGTTAGGAGAGGTGTTTTAGTGGTGTTTTAGTTTATGGGAGAGA
TTTAGATATTATTTTGTTGGGTTTTTAAGTTGGTATTTTAAATTTAGTAG
TTGATGAATTTAAGGAGTTTTTAAGTATTATTTGGTATGTTTTTTTGTAT
GTTTGTTTGTTTGTTTTTTGAGATAGAGTTTTTTTTTGTTATTTAGGTTG
GAGTGTAGTGACGTGATTTTGGTTTATTATAATGTTCGTTTTTTAGGTTT
AAGTGATTTTTTTGTTTTAGTTTTTTGAGTAGTTGGGATTATAGGTACGT
GTTATTATATTTGGTTAAGTTTTGTATTTTTAGTAGAGATGGGTTTTATT
ATGTTGGTTAGGATGGTTTCGATTTTTTGATTTTGTGATTTATTAGTTTT
GGTTTTTTAAAGTGTTGCGATTATAGGTGTGAGTTATTATATTCGGTTTA
TTTGGTATGTTTTATATTAATTGGAGTTAGGTTTAGAGTAGGGAGGTGGT
TAGATAGTGATTGTAATTTTTATTTTTTGTTTTGGTTAAATTAATATTTA
GATGGGATAGTGAGAGGAGATATTATATTTTGGTTATATATTATTAATTT
TTGTTTGATTATATATATATATATATATAGTCGTTTAGATTTATAAATAC
GTTTTTATTATTTTTTTTATTTTTTAGTGTTTGGGTGGAGGTTTTTAAGT
AGGAATGTTTTTTTAGAGGAGAGGGTGGGAGTGGAATGATGGGATTTTAG
AATTTTTTTATTTTATTTTTTTATTTTTAGATTTTAGTTTAGATGTAATT
TTGTTTTTATTATTTTGCGTGTACGTGTACGTATATATATATGTATATAT
ATAGTTTTAATATTTATATATAGATATTTATTTTTGATATTTATATATGA
ATTAAAAGATAAACGTTGGTTTATAAAGGAATATATATGTTATTTATATA
TTTTTAATTTTGTTTAGTGATGTTTTTTTTAGTATTATTGTATAGATTTT
AGATAATATAGATATATAGGTATTTTTTGTATATTGAGTTTTAGGTTATT
TGGTTGAGTTGTTTTTATTTTATTTTAGTTGTATATTTTTTTATATTTAT
TTTATAAAGGAAGAGTTCGTTTTTTTAATTATGTTTTTTATGGTTTTTAG
AGTATTTTTGTTTTAAATGTTGTTAATTTTTTTTTAGATTTAAGATTTTT
TTTTTTTAGATCGTTGGAGGTTTAATGTTAGTGTATAGGAGGAGATCGTT
TTTTATTATATTTTTTGATTTTTAGAGGTTTTTTTTAGGGTAGGGGTTTG
GTTAGGAAGATTGAAGTTATGACGTTTTTTTGTGTAAGGATAGGAAAGAT
GTTTGTGGGTTGGAGGGTAGTTATAGGGAAGTAGTTTTTTTTATTTTTTG
AGAAAGTATTTGGGAATAAGTATTTTTTTTAGGAAGTGATTTTTTATTAT
TTTTTTTTGATTTTATAGGTTTGGTGGTTTAAGTTTTAGGTTAGTTGATG
TTTTTTATTGGGATTAGTAGATAAGGTTATGATTTTGTAGTGATTTCGAT
TTTATAATTAGGTTAGGTTCGGGTTTTATGGTTAGAAGCGAGGTTTAGGC
GAGTGGTTAAGACGCGGTAGGATAGGAGGATGCGTTTGGTTGTTTTCGGT
TGTTGGACGCGGTTTGTAGTAGGG [symbol] [symbol] [symbol] TTTTATT
TAGGGTTTCGGGTGTTTAGTTTTTTTATTTTTTATTTTGGCGGTTTAAAG
GTAAGGTTTAGATTTTGGAGAGGTTAAAGTCGGGTTAAAATAATAGCGAA
GTTGGGATTGGAATAGGCGTTGGGGGTTTGCGTATGATTTTAGGTTTAGA
ATGCGATATTTTAGTGACGTCGGGGAATGAGTTGAGATGGAGTCGGAGAT
AGAGGTTGGGGTTTAGGACGAGGCGTTTGGGTATGGGATCGGATTTGAGA
CGGTTGCGGCGTTGGTGTTTTGGGTTGGATTAGATTCGAATTTAGAGCGG
AGCGAGGGTTAGAGTTAGGGTAAGAGTTCGAAGTTTAG [symbol] [symbol]
GATTTTATTATGAATGACGTTCGAGTAGG
GTTGGAATTCGAAGTTCGAACGTATTGGAAATGTAGTCGGACGACGGTTG
GGATTCGAGTGGGTTCGGGTTAGAAATGGGGATTTTAGAGTTTTATCGAG
GCGTTCGAGGTTAGAATTGAAGTTTGAGATTTAGGTAGGATTCGAGATAG
AGTTAGAGACGATATCGA [symbol] [symbol] TCGAAGGTAGAT
TCGGGGCGTAAATTTGTGTAGGTATTGGCGCGTTGTTTGGAGTTTTGGGT
TCGCGTCGTTGAGCGTCGGTAGGTTGGATTAGCGTTGCGCGTCGGAAGTG
TATTCGGGACGTCGGGGTTTTGGAGGAGAGATTATACGTCGTAAAAGTGG
TTTTCGAATGCGTTCGGGGTTTGTTTTCGGCGATATTGTTGGTTCGGCGG
TCGTAGTTTATTTGAAGTAGTTTATTTTGTAGAAGATTTTTTTGTTTTTG
ATGTAGTAGTTGTTTTGTTGTTTTAGCGACGTGCGATATACGGAGTATTC
GAGGTATCGTACGTGTTAGA [symbol] [symbol] ACGGGGCGGGG
ACGGAGGTCGGTTTAGCGCGGGTTTTTTTTATTTCGGGTTTTAGTTTTTT
CGGTTTTATTTATAGTTAGAGGCGTTGAAGTTTAGAAGGTGTATGCGATT
TTTTTATTTTTTTTTCGTAATATTGAGATTTAGGGAAATGGAAATAAATT
TTTTTTATTTTTTAGACGGAATTCGGGGGTTTAGGTAGATTTTAAGTTTT
GTTTAAAGTTTCGTAGTCGGGTAGTAGTGGAGTTAGGATTTGGAAGTAAG
AGGATTTGCGGTGTTTTTTTGTAGTTTTTTGCGTTGCGTTTTACGTTTCG
ATAATACGTACGTAATATTTATTTTTGTTTTATTTTGAGTAGATATCGGT
TTAGGATTTCGAGGTCGTAGTTGGAGTAGATGTTTTTGTTTGTAGACGGT
ACGGAGGAGGCGGTAGAGGGCGGTGAGTAGATAGATGGCGTGTTGGGCGT
ATATGGGGAGGTTTGATTTTCGTTTTTGTTTAGAGTTTTTGTTAGGGTTT
CGGCGTTAGATTGAGTTTGCGGCGGGGGAGAGAAGGAGAGGAGCGTTGAT
TCGGGTATTTAGAGTTGGGGTTGTTTTGGAGATAAATTTGGGGTTTTCGA
GTATTAATTGATAACGAAATTTTTTTAGGATAATGCGTCGTAGATTGAAG
GATAGGGATGGAAGGGTTTATTGGAGAGGAAGGGACGGTTTTAATTTTTA
ATACGCGTATTTTGG [symbol] [symbol] TTAATATTTTTTGTCG
GAAAATCGAGGTTTAGAGAGGGGCGGCGTTCGTCGGGAGTTTTTTAGAGC
GTGAGTAGTAGTTCGTGGTCGGAAAATTTGGTTAGGTTTTTAGGGTCGGG
TCGTTGGAGGATTGGGTATCGTAGGTAGGGTTAAACGGATTTATTATGGC
GGGCGGCGCGGTTTTTTTAAGATAGCGGGTGGTCGTTTTGTAGTCGGATT
TTGGTTGGGTTATTATTTGGGGGAGGGGGGGAGGGAACGTAGGCGGCGGC
GGTTGTTGAATTGGTTTCGTATAGTTTGAGTTTAGCGTTTTTTCGCGTTT
GTTATATAAATCGGCGTCGAATAATGAGTTTTAATTTTGTAGTGGGTATT
TAAAGTTTTTTTTTATAAAAGCGGTGTTTTTTTAATGAAGTAATTTGAAT
TTGGATTGGATTTTTTTTTTTTTTTTTTTTTTTTTTTTGTATTTAATTCG
TGGTTTTTGAAGTAATCGTTTAGTTTTTTTGTAATTTAAGTTTTTGAGGG
GGGAAGAAAAATACGCGTATATATATATAAATTATTTGTAATTAGA
Exemplary Primer Sequences for
Amplification of LHX6 Region of
Interest 2 (Chr9: 124985406-124989839):
Forward primer1:
(SEQ ID NO: 26)
TTGGGTTGTAGTGTAGTGTAGA
Reverse primer1:
(SEQ ID NO: 27)
AATTCGCCCTTTCCAAAACT
Forward primer2:
(SEQ ID NO: 28)
GAGAGCGTAGTTCGGGGTAA
Reverse primer2:
(SEQ ID NO: 29)
TCTCGCGAACTAAACGCTAAA
Forward primer3:
(SEQ ID NO: 30)
AATTTAATGGATCGCGAGGA
Reverse primer3:
(SEQ ID NO: 31)
CCCCAAATCAACAACCTCAT
Forward primer4:
(SEQ ID NO: 32)
GGTGTTGGGAGCGGTTAAAG
Reverse primer4:
(SEQ ID NO: 33)
AAAACTCATTATTCGACGCC
GABRA2
GABRA2 Region of Interest:
chr4: 46,391,392-46,391,695
5′pad = 250 3′pad = 250
Non-Modified GABRA2 Region
of Interest Sequence:
(SEQ ID NO: 34)
GAGAGATGGGTACTTCACGCCACAACCCCCTCAACCAAGACCACCTCCGC
AACCACAGCGGGAAAAGCTAGGGGCAGGCAGCCCACTTTTGCAATTACTC
TACATTACAGTGAACTGCGGTCCTCACGTCTTCTTTTCTTCACTCCCCTT
AACAAAATAAAATAAATAATTTCCCTTCCTGCCCACCCACGGTTGCCCAA
GACCAAGTCTTAGAGGCAGCCGGGTTCCGATCAAGTGCTGCGAAACGACG
CGTTTGACAGCTGCTCTGGAGCCGAGGATCACAATAAGAGAAGGCGCGTG
AGGCTCCGGCAGCAAACACCAGCCAGGCAGGCAGCGGTAATCACAGTGAG
CAAAGTTGAAGACTATAAAAGAGCCAGGCTAACGTGCTGCCGAGCAGGTG
TCCTGGATTTTATTGTAGTTGCAAATATGCTTCTGGTGATAAGTATCGAA
ATTATTTTTTAAGTGCGCTGGGGAGGAGGGAGGTGCGGGAATTGAGCCTC
CTCCGTTTCCAGGGAGCCTCTGAAAGTGGGGTGGGGGGAGATGAGGCATG
CACGCGAGGCAAAGACAAACAGGAAAGTGTCTCTATCGGGACCAACGTGT
GCGACCCTACCTGAAACGGCAAGCAGAATTCGGTGTTTTCTTCCTTTTGC
CCTGATCTTGACGAGATAGGAAACTTGGGAGAGAGAGAGAGAGAGAGAGA
GAGAGAGAGAGAGAGAGAGAGAGAGAGAGAGAGAGAGACCGAGACTGCAG
CAGCCAAGAGAGCGTGGAGCGATGGGCTGGTGGAAGCCGGAGAGGAGCGC
TAGG
Bisulfite-Modified GABRA2
Region of Interest Sequence:
(SEQ ID NO: 35)
GAGAGATGGGTATTTTACGTTATAATTTTTTTAATTAAGATTATTTTCGT
AATTATAGCGGGAAAAGTTAGGGGTAGGTAGTTTATTTTTGTAATTATTT
TATATTATAGTGAATTGCGGTTTTTACGTTTTTTTTTTTTTATTTTTTTT
AATAAAATAAAATAAATAATTTTTTTTTTTGTTTATTTACGGTTGTTTAA
GATTAAGTTTTAGAGGTAGTCGGGTTTCGATTAAGTGTTGCGAAACGACG
CGTTTGATAGTTGTTTTGGAGTCGAGGATTATAATAAGAGAAGGCGCGTG
AGGTTTCGGTAGTAAATATTAGTTAGGTAGGTAGCGGTAATTATAGTGAG
TAAAGTTGAAGATTATAAAAGAGTTAGGTTAACGTGTTGTCGAGTAGGTG
TTTTGGATTTTATTGTAGTTGTAAATATGTTTTTGGTGATAAGTATCGAA
ATTATTTTTTAAGTGCGTTGGGGAGGAGGGAGGTGCGGGAATTGAGTTTT
TTTCGTTTTTAGGGAGTTTTTGAAAGTGGGGTGGGGGGAGATGAGGTATG
TACGCGAGGTAAAGATAAATAGGAAAGTGTTTTTATCGGGATTAACGTGT
GCGATTTTATTTGAAACGGTAAGTAGAATTCGGTGTTTTTTTTTTTTTGT
TTTGATTTTGACGAGATAGGAAATTTGGGAGAGAGAGAGAGAGAGAGAGA
GAGAGAGAGAGAGAGAGAGAGAGAGAGAGAGAGAGAGATCGAGATTGTAG
TAGTTAAGAGAGCGTGGAGCGATGGGTTGGTGGAAGTCGGAGAGGAGCGT
TAGG
Exemplary Primer Sequences for
Amplification of GABRA2 Region
of Interest:
Forward primer:
(SEQ ID NO: 36)
TCGGGTTTCGATTAAGTGTTG
Reverse primer:
(SEQ ID NO: 37)
TTTCAAATAAAATCGCACAC

In some embodiments, the biomarker panel is a microarray.

In some embodiments, the primers or probes are biotinylated and bind to streptavidin coated substrates for selection of the DMPs or DMP-associated genes targeted by the probe or primers. In some embodiments, the streptavidin-coated substrates are beads.

Described herein are methods to assay the methylation status of DMPs or DMP-associated genes described herein to diagnose or give a prognosis for hypersomnolence, idiopathic hypersomnia, or long sleep time in an individual. Methylation levels of at least one DMP or DMP-associated gene recited in Tables 1 and 2, or any combination of DMPs or DMP-associated genes recited therein, is measured in target DNA from a biofluid sample from a subject.

Methylation may be quantified by any suitable means known in the art. Suitable methods for assaying quantification are disclosed, for example, by Kurdyukov and Bullock (“DNA methylation analysis: Choosing the right method,” Biology, 2016, 5(3)). Suitable methods for quantifying or assaying methylation may include, but are not limited to methylation specific polymerase chain reaction (PCR), high resolution melting, cold-PCR, pyrosequencing, PCR and sequencing, bead array, and digestion-based assay followed by PCR or quantitative PCR (qPCR).

In some embodiments, the target DNA is bisulfite modified. Bisulfite treatment mediates the deamination of cytosine to uracil, whereby the modified uracil residue will be read as a thymine as determined by PCR-amplification and sequencing. Methylated cytosine (5mC) resides are protected from this conversion and will remain as cytosine.

To examine the methylation status of the DMP or DMP-associated gene, target genomic DNA may be isolated from a biofluid sample from a subject. In some embodiments, the target DNA is isolated from a biofluid sample from a human.

As used herein, “biofluid” refers to a biological fluid produced by a subject. Suitable biofluids include, but are not limited to blood, saliva, sweat, urine, semen, tears, and cerebrospinal fluid. In some embodiments, the biofluid is selected from the group consisting of blood, saliva, sweat, urine, semen, tears, and cerebrospinal fluid. In some embodiments, the biofluid is blood. In some embodiments, the biofluid is saliva.

Following isolation of target DNA, the target DNA will be contacted with probes specific to the DMPs outlined in Tables 1 and 2 to isolate and enrich these genomic regions from the target DNA sample. In some embodiments, sequences of the DMP is used as bait to isolate the genomic regions of interest for amplification and sequencing.

After isolation and enrichment of the genomic regions within the target DNA that include the DMP, methylated adapters are ligated to the enriched regions. The sample with the ligated methylated adapters may then be subject to sodium bisulfite modification.

In some embodiments, the target DNA sample is bisulfite modified without first enriching the sample of the genomic regions of the DMPs. In some embodiments, the target DNA is first bisulfite modified then enriched for the genomic regions of the DMPs.

In general, target DNA or bisulfite modified target DNA is subject to amplification. The amplification may be polymerase chain reaction (PCR) amplification. PCR amplification will include single or multiple pair(s) of primers and probes at specific DMPs outlined in Tables 1 and 2. The target DNA amplification and methylation quantification will be evaluated in one or multiple tubes.

In some embodiments, methylation is quantified by amplification and sequencing of target DNA. Bisulfite modified target DNA may be subject to PCR to amplify target regions comprising DMPs outlined in Tables 1 and 2. The PCR reaction mixture typically includes at least one pair of primers designed to target a DMP detailed in Tables 1 and 2, PCR buffer, dNTPs (e.g., adenine, thymine, cytosine and guanine), MgCl₂, and polymerase. PCR amplification generally includes the steps of heating the reaction mixture to separate the strands of the target DNA, annealing the primers to the target DNA by cooling the reaction mixture, allowing the polymerase to extend the primers by addition of NTPs, and repeating the process at least 2, at least 5, at least 10, at least 15, at least 20, at least 25, or at least 30 times to produce a PCR amplification product. If the target DNA in the reaction mixture is single stranded, the initial heating step may be omitted, however this heating step will need to be included when the second and subsequent times the reaction is completed to separate the extended primer strands from the opposite strand and DNA (e.g., the target DNA or another previously extended primer strand). In some embodiments, the target DNA is bisulfite modified prior to amplification.

In some embodiments, the bisulfite modified target DNA is used in a methylation-specific-quantitative PCR (MS-QPCR) reaction such as MethylLight (WO 2000/070090A1) or HeavyMethyl (WO 2002/072880A2). For example, a reaction mixture for use in a MethylLight methylation specific PCR reaction would contain primers and probes specific to the DMPs recited in Tables 1 and 2, PCR buffer, dNTPs (e.g., adenine, thymine, cytosine and guanine), MgCl₂, and polymerase. A typical kit for methylation specific PCR may include primers and probes specific to the DMPs recited in Tables 1 and 2, wild type reference gene primers such as β-actin, PCR buffer, dNTPs, MgCl₂, polymerase, positive and negative methylation controls, and a dilution reference. The MS-QPCR may be carried out in one or multiple reaction tubes.

In some embodiments, either the forward or reverse primer of the primer pair used in the PCR amplification reaction is biotinylated. When a biotinylated primer is used in a PCR amplification reaction, PCR products may be purified, captured, and/or sorted with a streptavidin coated substrate. In some embodiments, the substrate is a streptavidin coated bead. In some embodiments, the beads are streptavidin sepharose beads. In some embodiments, the beads are magnetic.

In some embodiments, the PCR amplification product is contacted with one or more probes specific for and complementary to a DMP detailed in Tables 1 and 2. The probe may be biotinylated. The PCR amplification product and probe mixture can then be purified, captured and/or sorted with a streptavidin coated substrate. In some embodiments, the substrate is a streptavidin coated bead. In some embodiments, the beads are streptavidin sepharose beads. In some embodiments, the streptavidin beads are magnetic.

In some embodiments, methylation is quantified using pyrosequencing. Bisulfite modified target DNA may be subject to PCR to amplify target regions comprising DMPs outlined in Tables 1 and 2 as described above. PCR amplification products are purified, denatured to single-stranded DNA, and annealed to a sequencing primer for methylation quantification by pyrosequencing of the DMP or DMP-associated gene as detailed in Tables 1 and 2. In some embodiments, methylation may be quantified with PyroMark™MD Pyrosequencing System (Qiagen) using PyroPyroMark® Gold Q96 Reagents (Qiagen, Cat# 972804) (QIAGEN PyroMark Gold Q96 Reagents Handbook 08/2009, 36-38).

In some embodiments, bisulfite treated DNA is subject to an Invader® assay to detect changes in methylation. The Invader® assay entails the use of Invader® chemistry (Hologic Inc.; invaderchemistry.com; Day, S., and Mast, A. Invader assay, 2004; Chapter in Encyclopedia of Diagnostic Genomics and Proteomics. Marcel Dekker, Inc., U.S. Pat. Nos. 7,011,944; 6,913,881; 6,875,572 and 6,872,816). In the Invader® assay, one would use a structure-specific flap endonuclease (FEN) to cleave a three-dimensional complex formed by hybridization of C/T specific overlapping oligonucleotides to target DNA containing a CG site. Initial PCR amplification of the bisulfite treated target DNA may be necessary if the quantity of the bisulfite treated target DNA is less than 20 ng.

In some embodiments, the methylation status is measured by methylation-specific PCR, quantitative methylation-specific PCR, methylation-sensitive DNA restriction enzyme analysis, or bisulfite genomic sequencing PCR, or quantitative bisulfite pyrosequencing. See, e.g., U.S. Pat. Nos. 10,370,726 and 10,934,592, which are incorporated herein by reference in their entireties

Methylation status of the DMPs and DMP-associated genes described herein may be useful in identification and management of both acute and chronic sleep problems in a subject. Assays described herein are useful for identifying insufficient sleep time in persons complaining of daytime sleepiness improving patient care in sleep medicine. The assays described can also be used as to quantify change in sleep duration in research and clinical care (e.g., identifying increased sleep time caused by a medication; clarifying whether a behavioral intervention increases sleep time in people who are chronically sleep restricted, etc.) or in general assessments of sleep time in a subject.

For applications in general wellness assessments of subject (e.g., a healthy subject), the methylation assays to evaluate sleep in a subject as described herein can be used to optimize mental performance, to optimize physical performance, to optimize recovery after mental exertion, to optimize recovery after physical exertion, to optimize academic performance, to help control weight, to help reduce weight, to help reduce obesity, to help control acute pain, to help control chronic pain, and to prevent the onset of medical, psychiatric, and neurological disease.

Methods described herein for assaying methylation to evaluate sleep in a subject as described herein may be used to increase or optimize fecundity or fertility in a subject. Likewise, the methods described herein for assaying methylation to evaluate sleep in a subject as described herein may be used to aid in a subject's general wellness during pregnancy. The methods described herein for assaying methylation to evaluate sleep in a subject may be used to aid in post-partum recovery and mental and physical wellness post-partum.

In some embodiments, the methods described herein for assaying methylation to evaluate sleep in a subject may be used to increase or optimize wound healing. The methods may also help in physical or mental trauma recovery in an individual or in post-surgical recovery.

In some embodiments, the methods described herein for assaying methylation may be used to evaluate sleep duration and sleepiness in a subject. In some embodiments, the methods described herein for assaying methylation may be used to evaluate sleep duration and sleepiness in a subject diagnosed with a sleep disorder (e.g., insomnia, restless legs syndrome, circadian rhythm sleep-wake disorders, sleep apnea, and parasomnias).

In some embodiments, methods described herein for assaying methylation may be used to evaluate sleep duration in a subject with a medical or neurological disorder (e.g., cancer, coronary heart disease, atrial fibrillation, congestive heart failure, epilepsy/seizure disorders, thyroid dysfunction, Parkinson's disease, fibromyalgia, Myotonic dystrophy, Kline Levin Syndrome, rheumatologic disease such as systemic lupus erythematosus, renal disease, hepatic disease such as non-alcoholic fatty liver disease, diabetes, hypertension, and asthma). In some embodiments, methods described herein for assaying methylation may be used to evaluate sleep duration in a subject with a psychiatric disorder (e.g., mood, thought, attentional, psychotic, personality, anxiety, and PTSD)

Assaying methylation status of the DMPs and DMP-associated genes described herewith will be beneficial as a measure of habitual sleep duration to help subjects identify if they are sleeping too little or too much to maintain personal wellness. In some embodiments, assays for determining the methylation status of DMPs or DMP-associated genes described herein may be incorporated into a person fitness tracker and the target DNA may be from sweat.

Assaying methylation status of the DMPs and DMP-associated genes described herein may also be beneficial for use in employee health screening (e.g., to determine if an employee is at risk for chronic disease), for use in actuarial sciences and risk estimation (e.g., to determine if an individual has sleep insufficiencies or excessive sleep times that would be associated with increased mortality), to monitor sleep and its impact on chronic disease treatment (e.g., identifying and characterizing sleep duration can improve management of chronic illnesses such as diabetes, asthma, and hypertension), identification of individuals who may be at risk for psychological disorders or mental illness (e.g. too much or too little sleep are associated with and may precede major depressive disorder, manic episodes, etc. allowing for targeted intervention strategies to prevent negative mental illness outcomes), and to determine the factors associated with attention and cognitive disorders (e.g. characterization of sleep loss as a contributing factor to attention-deficit hyperactivity disorder and Alzheimer's disease).

Methylation status of the DMPs and DMP-associated genes described herein may also be beneficial in occupational health and safety applications. For example, identification of people who are sleeping too little to safely perform their required occupational duties (e.g., airplane pilots, bus/train drivers, long haul truckers, etc.), forensic applications (e.g., determining if reduced sleep duration or excessive sleep preceding an accident was a contributing factor in the accident, etc.), and in law-enforcement (e.g., analysis of compliance or non-compliance with fatigued-driver laws where it is a crime to drive if a person has been awake for greater than 24 hours, etc.).

In some embodiments, the methods described herein additionally include a step of treating or providing treatment recommendations to a subject diagnosed with hypersomnolence, idiopathic hypersomnia, or long sleep time. Suitable treatments include administering to a subject a wake promoting medication or stimulant (e.g., amphetamines, modafinil, etc.), administering to a subject an oxybate or derivative thereof (e.g., sodium oxybate), or having the subject undergo sleep extension therapy (e.g., sleeping 1-2 hours longer to see if this improves symptoms). In some embodiments, suitable treatments may include changing one or more sleep practices to improve sleep quality (e.g., amount of light, ambient noise, temperature, etc.), changing the time of day a subject sleeps (e.g., going to bed earlier or staying up later), having a subject nap more or less during the day, or administrating to a subject one or more over the counter sleep promoting aids such as melatonin.

The genomic positions described herein refer to genomic positions as provided in the UCSC hg19 human reference genome (Fujita P A, Rhead B, Zweig A S, Hinrichs A S, Karolchik D, Cline M S, Goldman M, Barber G P, Clawson H, Coelho A, Diekhans M, Dreszer T R, Giardine B M, Harte R A, Hillman-Jackson J, Hsu F, Kirkup V, Kuhn R M, Learned K, Li C H, Meyer L R, Pohl A, Raney B J, Rosenbloom K R, Smith K E, Haussler D, Kent W J. The UCSC Genome Browser database: update 2011. Nucleic Acids Res. 2011 January; 39(Database issue):D876-82. doi: 10.1093/nar/gkq963.) and positions in other genomes aligning thereto. Suitable alignment methods are known in the art. Alignments are typically performed by computer programs that apply various algorithms, however it is also possible to perform an alignment by hand. Alignment programs typically iterate through potential alignments of sequences and score the alignments using substitution tables, employing a variety of strategies to reach a potential optimal alignment score. Commonly-used alignment algorithms include, but are not limited to, CLUSTALW, (see, Thompson J. D., Higgins D. G., Gibson T. J., CLUSTAL W: improving the sensitivity of progressive multiple sequence alignment through sequence weighting, position-specific gap penalties and weight matrix choice, Nucleic Acids Research 22: 4673-4680, 1994); CLUSTALV, (see, Larkin M. A., et al., CLUSTALW2, ClustalW and ClustalX version 2, Bioinformatics 23(21): 2947-2948, 2007); Jotun-Hein, Muscle et al., MUSCLE: a multiple sequence alignment method with reduced time and space complexity, BMC Bioinformatics 5: 113, 2004); Mafft, Kalign, ProbCons, and T-Coffee (see Notredame et al., T-Coffee: A novel method for multiple sequence alignments, Journal of Molecular Biology 302: 205-217, 2000). Exemplary programs that implement one or more of the above algorithms include, but are not limited to MegAlign from DNAStar (DNAStar, Inc. 3801 Regent St. Madison, Wis. 53705), MUSCLE, T-Coffee, CLUSTALX, CLUSTALV, JalView, Phylip, and Discovery Studio from Accelrys (Accelrys, Inc., 10188 Telesis Ct, Suite 100, San Diego, Calif. 92121). In a non-limiting example, MegAlign is used to implement the CLUSTALW alignment algorithm with the following parameters: Gap Penalty 10, Gap Length Penalty 0.20, Delay Divergent Seqs (30%) DNA Transition Weight 0.50, Protein Weight matrix Gonnet Series, DNA Weight Matrix IUB.

The genomic positions of any CpG sites described herein refer to the position of the C in the CpG site in the original genome.

Unless defined otherwise, all technical and scientific terms used herein have the same meaning as commonly understood by one of ordinary skill in the art to which the invention pertains. All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein, the terms “approximately” or “about” in reference to a number are generally taken to include numbers that fall within a range of 5% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

The present invention has been described in terms of one or more preferred embodiments, and it should be appreciated that many equivalents, alternatives, variations, and modifications, aside from those expressly stated, are possible and within the scope of the invention.

### EXAMPLES

### Background

Hypersomnolence, broadly defined as excessive daytime sleepiness often accompanied by prolonged sleep duration, is one of the most common symptom constellations encountered in persons with sleep disorders. While the pathophysiology of hypersomnolence can be identified in some cases, for example orexin deficiency in type 1 narcolepsy,¹ the cause of hypersomnolence cannot universally be determined for many patients despite comprehensive evaluation, resulting in ambiguous diagnoses and a lack of targeted therapies to improve symptomatology. Thus, there is a clear need to better understand the biological basis of unexplained hypersomnolence to advance clinical care through the development of advanced diagnostics and personalized therapeutics for these patients.

One of the great challenges that must be overcome in this area of research is the fact that hypersomnolence is a multifaceted symptom, for which no singular objective measure is able to fully capture the subjective complaint.²⁻³ It is widely appreciated that the multiple sleep latency test (MSLT), while highly useful in confirming type 1 narcolepsy, has limited test-retest reliability in disorders of unexplained hypersomnolence.⁴⁻⁶ While the MSLT remains a key tool in the practice of sleep medicine, it quantifies sleep propensity during repeated nap opportunities and the presence of sleep onset REM periods, but does not capture other relevant aspects of hypersomnolence. Supplementary measures frequently capture other key facets of hypersomnolence, including the maintenance of wakefulness test, which quantifies the ability to remain awake under soporific conditions, infrared pupillometry, which quantifies drowsiness under resting conditions, the psychomotor vigilance task, which quantifies neurobehavioral alertness, and extended duration polysomnographic recordings, which measure excessive sleep duration. While these aspects of hypersomnolence correlate with subjective clinical complaints, they only marginally explain the variance of one another.⁷⁻⁹ Thus, the measurable phenotype in patients with unexplained hypersomnolence can be quite complex, with variable combinations of abnormalities occurring among individual patients.⁷⁻⁹

Since there is heterogeneity among objective measurable deficits in persons with hypersomnolence, it is highly likely that there is also significant heterogeneity in the underlying biology associated with clinical complaints. Sleep medicine has traditionally focused on research that compares various disorders of excessive sleepiness defined by the International Classification of Sleep Disorders (ICSD) both against one another and healthy persons. The diagnostic criteria for disorders such as idiopathic hypersomnia have shifted over time, often with limited clinical or biological data to support alterations in nosology. To disentangle the undoubtedly complex causes of unexplained central nervous system hypersomnolence requires that research efforts focus on the biology of specific and measurable phenotypes that may cut across traditional diagnostic boundaries, rather than nosological distinctions defined largely by expert opinion.¹⁰

The importance of using refined and objective phenotypes is particularly salient for the study of the genetics that may be related to hypersomnolence complaints. Effort has been made to utilize genome-wide association studies (GWAS) to identify candidate single nucleotide polymorphisms (SNPs) that are associated with various sleep phenotypes.¹¹ Unfortunately, the SNPs that are statistically related to sleep measures in these large-scale and hypothesis-free investigations often have modest effect sizes on the overall phenotype, suggesting that genetic variation alone is unlikely to account for extremes of daytime sleepiness and/or sleep duration observed in clinical practice.¹²⁻¹⁶ Additionally, the sleep phenotype used in GWAS studies is almost universally based on self-report, which may be both inaccurate and have limited connection to relevant underlying genetic variability. Thus, while GWAS can help identify potential genes linked to broad phenotypic traits, alternative approaches are needed to connect findings from large-scale population-based GWAS investigations to smaller populations of heterogeneous and disordered patients.

In this context, epigenetics, the study of molecular modifications not affecting the genetic code itself, may be highly applicable to the study of unexplained hypersomnolence. Employing epigenetic methodologies is a crucial step in clarifying how candidate genes are related to specific observable and measurable phenotypes in sleep disorders. In particular, combining epigenetics with a detailed and objective phenotyping approach is likely to advance our understanding of how specific gene regulatory elements are related to the pathophysiology of human disease. Therefore, the present examples examined alterations in DNA methylation, a key component of epigenetic regulation, in a well-characterized sample of patients with unexplained hypersomnolence, to determine if epigenetic modification of candidate genes were related to specific aspects of the hypersomnolence phenotype.

### Methods

### Participants

All participants were clinical patients referred by their treating clinician for sleep testing to evaluate complaints of hypersomnolence at Wisconsin Sleep, the sleep clinic and laboratory affiliated with the University of Wisconsin-Madison. For inclusion, participants had to be 1) free of psychotropic medications at the time of and preceding sleep procedures, 2) not have any identifiable cause of hypersomnolence [e.g., sleep apnea defined as apnea hypopnea index (AHI)>5/hr), evidence of sleep deprivation prior to sleep testing (using either sleep logs and/or actigraphy), complaints of cataplexy suggesting type 1 narcolepsy, etc.], and 3) have no sleep onset REM periods on either overnight polysomnography (PSG) or MSLT. Participants provided informed consent for all study procedures, including both sleep phenotyping measures not part of routine clinical care, as well as collection of biospecimens for subsequent genetic analysis.

### Hypersomnolence Phenotyping Procedures

The hypersomnolence phenotype was determined using four parameters: 1) mean sleep latency on MSLT, 2) total sleep time on ad libitum polysomnography, 3) pupillary unrest index measured by infrared pupillometry, and 4) lapses on the psychomotor vigilance task. All measures were collected within a single sleep laboratory visit.

Participants arrived at the sleep laboratory for testing at approximately 19:00-20:00. After polysomnographic set-up, participants determined their bedtime and were only disturbed after sleep onset if technical issues arose with the recording that would inhibit sleep scoring/staging. The end of the PSG recording was determined by the patient informing the technician that they were ready to get up for the day, rather than a universally applied standard wake time. An MSLT was subsequently performed, with sleep latency defined as the time from lights out to the first 30-second epoch scored as any stage of sleep. The nap was terminated after 20 minutes (if no sleep was achieved) or 15 minutes after the first epoch of scored sleep.¹⁷ Both PSG and MSLT were collected using Alice Sleepware (Phillips Respironics, Murrysville, Pennsylvania, United States) and scored following standard criteria.¹⁸

Following MSLT naps 1 and 3, the pupillographic sleepiness test (PST) and the psychomotor vigilance task were collected with values at each timepoint averaged. The PST is an assessment of drowsiness under constant darkness that records oscillations of the pupil diameter via a computer-based infrared video technique.²⁵ These undulations result from progressive reduction of noradrenergic central activation from the locus coeruleus, resulting in disinhibition of the parasympathetic Edinger-Westphal nucleus.¹⁹ The fluctuations in pupil diameter are utilized to calculate the pupillary unrest index (PUI), defined by absolute values of cumulative changes in pupil size based on the mean values of consecutive data sequences,²⁰ with higher values suggestive of increased drowsiness. Reproducibility, reliability, and normative values of the PUI have been established for adults.^{19,21-23} The PSTEco system (AMTech, Germany) was used for ascertainment of PUI in the present examples and was applied following established protocols.¹⁹

The 10-minute psychomotor vigilance task (PVT) was collected after PST to minimize the potential impact of time-on-task effects from this measure of neurobehavioral alertness on PUI. The PVT is a well-validated measure used in sleep research that quantifies the ability to sustain attention and respond in a timely manner to salient signals.²⁴ The PVT requires responses to a stimulus (digital counter) by pressing a button as soon as the stimulus appears, which stops the stimulus counter and displays the reaction time in milliseconds for a 1-second period. The number of lapses (failure to respond within 500 msec of stimulus; Tukey transformed) was considered the primary PVT measure of interest in the present examples.

### DNA Extraction and Methylation Detection

Saliva samples were collected from participants approximately two hours after awakening using Oragene kits for DNA methylation analysis (DNA Genotek, Canada). DNA was extracted following the manufacturers protocol. Genomic DNA samples were resolved on a 1% agarose gel, to verify the DNA was of high molecular weight, and quantified using Qubit (Qiagen, USA). Genome-wide DNA methylation levels were determined using the HumanMethylationEPIC array and raw intensity data files were imported into the R package minfi to assess sample quality, calculate the detection P-value of each tested probe, filter probes, and determine beta values.²⁵ Probes were background- and control-corrected, followed by subset-quantile within array normalization to correct for probe-type bias.²⁵⁻²⁷ Probes were removed from further analysis if: one sample or more exhibited a detection P-value>0.01; the probe contained a known single nucleotide polymorphism (SNP); the probe was derived from a sex chromosome; the probe measured methylation at a cytosine followed by a nucleotide other than guanine; or the probe was a cross-reactive probe. With these filtration criteria, 97,964 probes were discarded and 768,127 probes were available for further analysis.

### Statistical Analyses

Methylation levels (i.e., beta-values) were calculated in minfi as the ratio of methylated to total signal (i.e., beta-value=methylated signal/[methylated signal+unmethylated signal+100]), where beta-values range from 0 (unmethylated) to 1 (methylated). Beta values were further converted to M-values (i.e., logit-transformed beta-values) for differential analysis, as M-values are more appropriate for statistical testing. Individual models for each independent objective variable of interest [i.e., average PUI on infrared pupillometry, total sleep time (TST) on PSG, mean sleep latency (MSL) on MSLT, and average PVT lapses] were generated, while also accounting for the dependent variables of age, sex, body mass index (BMI), and HumanMethylationEPIC Beadchip identification number. Score on the Epworth Sleepiness Scale (ESS) was similarly evaluated as a subjective measure of daytime sleepiness for comparison purposes.' Since saliva tissues may be confounded due to latent factors such as heterogeneous cell populations, the R package sva was employed to identify any surrogate variables not accounted for in the model.²⁹ In each model, one surrogate variable was identified and adjusted for in the model using sva. Linear regression for each tested CpG using a multivariate model was employed using the R package limma.³⁰

To assess systematic bias of the linear regression model, the genomic inflation factor was calculated for the obtained P-values for each model, yielding a genomic inflation factor of: PUI (λ=0.99), TST (λ=1.05), MSL (λ=1.05), PVT (λ=1.37), and ESS (λ=1.19). In the latter two cases (PVT and ESS) the slightly elevated λ score suggests modest inflation of the generated P-values, indicative of potential bias in the model. As such, these two models were reanalyzed without adjusting for surrogate variables and the genomic inflation factor was reassessed. Without surrogate variable adjustment, the genomic inflation factors were: PVT (λ=0.956) and ESS (λ=0.76), indicating a more unbiased approach without adjusting for surrogate variables in the cases of the independent variables PVT and ESS. In addition, the R package NHMMfdr used here reports a BIC score based on the P-values generated from model fitting for both the adjusted and unadjusted models. Model fitting in NHMMfdr resulted in a lower BIC score using the unadjusted models for PVT and ESS. Together, these data supported that differential methylation modeling should not adjust for the identified surrogate variable for the PVT and ESS variables. Notably, the deflated lambdas for these two cases would result in type II error (i.e., false negative), and may be caused by population stratification in our sample, potentially stemming from the higher proportion of female to male participants.

The methylation levels of immediately flanking probes tested by array-based platforms often exhibit dependence upon one another, and because corrections for multiple testing such as the Benjamini-Hochberg false discovery rate may be inefficient under a varying dependence structure, the R package NHMMfdr was used to detect the adjusted local index of significance (aLIS), an extension of adjusted P-values, for each probe by first converting all P-values to z-scores, followed by employing a Hidden Markov Model to determine the “aLIS P-value.”' An aLIS P-value threshold of <0.05 was used to identify significant differentially methylated loci.

### Results

### Participant Demographics and Phenotype

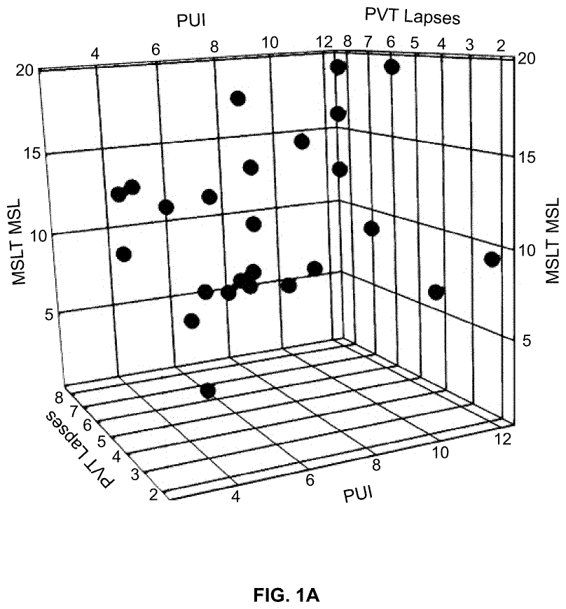

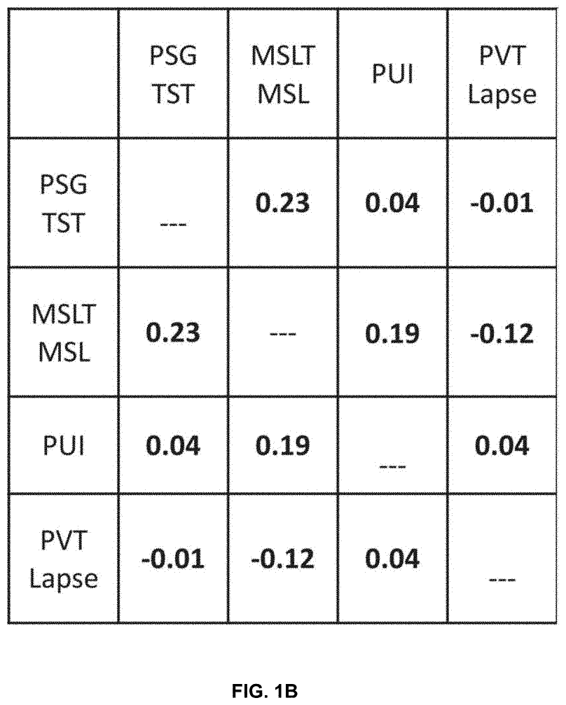

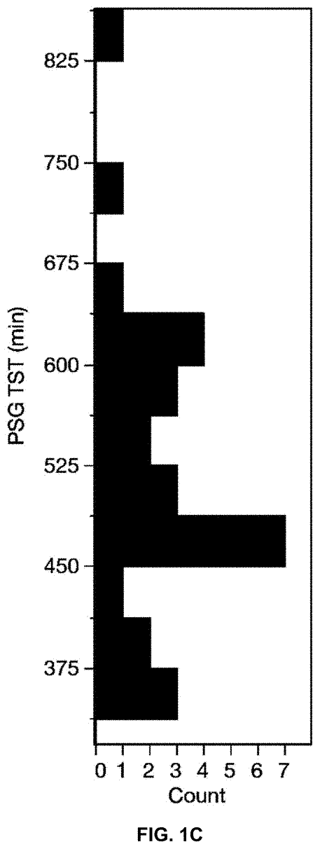

A summary of the 28 patients included in these analyses is provided in Table 3. The sample was predominantly (75%) female. While the mean age was 31.7±11.9 years, the overall range was relatively broad (20 to 66 years). The mean Epworth Sleepiness Scale score was 12.8±3.6 for the sample. Overall, there also was a wide range of values for each hypersomnolence phenotypic trait of interest (e.g., TST, MSL, PUI, and PVT) across the sample, with minimal correlation between values for any objective measure (FIGS. 1A-1C).

TABLE 3
Demographics and Sleep Phenotypic Data for the Sample (N = 28).

| Characteristic | Value | |
|---|---|---|
| Female Sex, n (%) | 21 | (75) |
| Age in years, mean (SD) | 31.7 | (11.9) |
| BMI in kg/m², mean (SD) | 26.2 | (5.3) |
| MSLT MSL in minutes (SD) | 12.8 | (4.2) |
| PSG TST in minutes (SD) | 521.7 | (112.4) |
| PUI (SD) | 5.9 | (2.5) |
| Lapses, mean (SD) | 2.5 | (1.4) |

PAX8/PAX8-AS1 DNA Methylation Levels are Associated with Sleep Duration

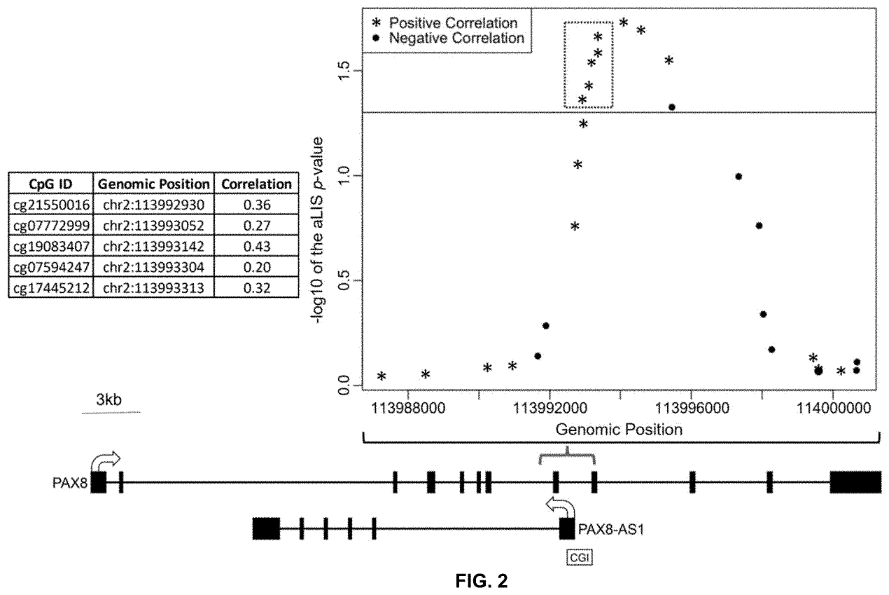

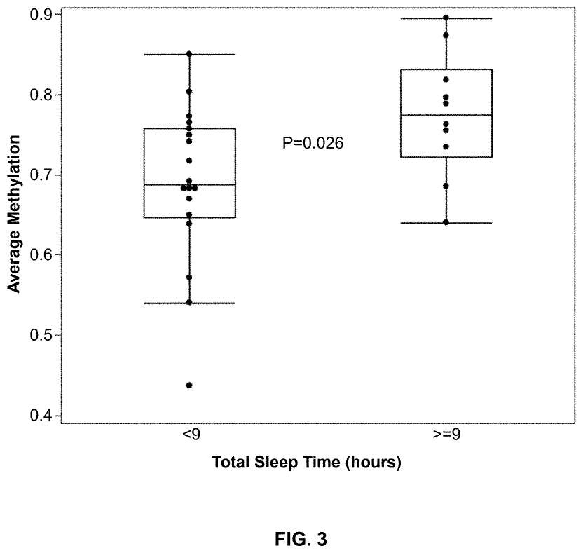

We first took a hypothesis-driven approach by examining the DNA methylation levels of eleven genes previously associated with the hypersomnolence phenotype (Table 4). Genomic DNAs from saliva were examined using the HumanMethylationEPIC beadchip array, which provides a quantitative measure of DNA methylation levels at 866,091 CpG/CpH dinucleotides across the genome at single-nucleotide resolution, including enhancers and all coding regions. Initial differential methylation analyses centered only on the 832 CpG sites annotated to these eleven genes and utilized independent regression models of the 5 hypersomnolence scores as the explanatory variable (Methods). These analyses revealed that only 9 CpG sites exhibited significant associations between DNA methylation levels and any of the 5 hypersomnolence measures (Table 1). All 9 differentially methylated CpG sites were annotated to the paired box 8 (PAX8) gene and its related antisense gene (PAX8-AS1) and these methylation levels were correlated to total sleep time measured using ad libitum PSG (aLIS P-value<0.05). Among these 9 differentially methylated positions (D1VIPs) was a notable cluster of 5 CpG sites within a small (˜400 base pair) region located in the body of the PAX8 gene and promoter of PAX8 AS1 (FIG. 2). All five DMPs within this cluster had a positive correlation (mean 0.31±0.09) with sleep duration (i.e., increasing methylation was associated with longer sleep time). To quantify the magnitude of the effect of methylation of the 5 DMPs, the sample was also stratified into those with TST<and ≥9 hours.^{32,33} Participants with hypersomnia had mean methylation of these 5 DMPs that was 8.6% greater than those without excessive sleep duration (77.4% vs. 68.6%; P-value=0.026; FIG. 3).

TABLE 4
A priori list of genes contributing to hypersomnolence.

| | | aLIS | |
|---|---|---|---|
| Gene | Name | P-value | Ref |
| PDE4D | Phosphodiesterase 4D | N.S. | 57, 58 |
| NPR2 | Natriuretic peptide receptor 2 | N.S. | 59 |
| SP140 | Speckled protein 140 | N.S. | 59 |
| PAX8 | Thyroid-specific transcription factor | 0.021 | 12, 13 |
| | paired box gene 8 | | |
| IL1RN | Interleukin-1 receptor antagonist | N.S. | 12, 60 |
| AD0RA2A | Adenosine A(2) receptor | N.S. | 61, 62 |
| AR | Androgen receptor | N.S. | 13 |
| OPHN1 | Oligophrenin-1 | N.S. | 13 |
| PER3 | Period 3 | N.S. | 62, 63 |
| CACNA1C | Calcium voltage-gated channel subunit | N.S. | 64-66 |
| | Alpha1 C | | |
| ZFYVE28 | Zinc Finger FYVE-Type Containing 28 | N.S. | 67 |

Genome-Wide DNA Methylation Levels are Associated with Other Aspects of the Hypersomnolence Phenotype

We additionally conducted genome-wide exploratory comparisons of the 5 independent continuous variables of hypersomnolence scores as the explanatory variable (Methods), which each yielded a unique set of DMPs. Notably, DMPs were found in two genes. First, LHX6 contained 11 DMPs associated with mean sleep latency on the MSLT (Table 2). Among these 11 DMPs was a notable cluster of 6 CpG sites within a small (˜500 base pair) region that were located in the body of the LHX6 gene. All six DMPs within this cluster had a negative correlation with MSLT MSL (i.e., increasing methylation was associated with reduced sleep latency). Second, GABRA2 contained 7 DMPs associated with PUI using infrared pupillometry (Table 2). These 7 DMPs were within a small (˜300 base pair) region located in the promoter of the GABRA2 gene. The majority (6/7) of these DMPs had a negative correlation with sleep duration (i.e., decreasing methylation was associated with increasing PUI). Significant findings were not found using either ESS or PVT as the independent variable in either the hypothesis-driven or genome-wide analyses.

### Discussion

The present examples demonstrate that PAX8/PAX8-AS1 DNA methylation levels are associated with sleep duration quantified using in-laboratory ad libitum polysomnography in a well-characterized, unmedicated group of clinical patients with unexplained hypersomnolence. Specifically, 9 differentially methylated sites within PAX8/PAX8-AS1 were associated with sleep duration, of which 5 formed a distinct cluster of CpG sites within a small (-400 base pair) region in the body of the PAX8 gene and promoter of PAX8-AS1. Since antisense genes frequently play key roles in reducing expression of overlapping sense genes and form self-influencing circuits with regulatory advantages over transcription factor proteins', the location and clustering of these significantly differentially methylated CpG sites increase the likelihood they may directly impact both PAX8 and PAX8 AS1 expression. In fact, more than 70% of the transcripts in humans have antisense transcripts that are generated by independent promoters.' Antisense transcript can function by the act of its own transcription in cis (controlling genes locally on the DNA strand involved in its origination), which regulates sense gene expression by interfering/blocking sense transcriptional machinery.'

While the specific determinants of habitual sleep time remain unknown, recent genome wide association studies (GWAS) have highlighted the role of PAX8/PAX8-AS1 in human sleep duration. PAX8 likely serves several roles as a master transcription factor crucial to embryonic development and maintenance functions after birth.³⁸ Using both self-report and accelerometer data in population-based cohorts, SNPs in PAX8IPAX8-AS1 have repeatedly demonstrated significant effects on sleep duration.¹²⁻¹⁶ However, the SNPs themselves impact sleep duration only marginally (i.e., only a few minutes longer sleep per day). Thus, while hypothesis-free GWAS investigations have pointed to a role of PAX8/PAX8-AS1 in habitual sleep duration, specific sequence variability in PAX8/PAX8-AS1 is unlikely to be a major contributing factor in pathological hypersomnia. In the present examples, we have further extended these well-replicated associations between PAX8/PAX8-AS1 and sleep duration by finding a significant association between PAX8/PAX8-AS1 DNA methylation levels and nocturnal sleep duration in symptomatic patients. The magnitude of the effect between sleep duration and PAX8/PAX8-AS1 methylation levels (e.g., —10%) observed in the present examples is on par with methylation level changes that are the epigenetic hallmark of complex disease phenotypes.³⁹ Unique to our approach was the application of deep phenotyping¹⁰, which carefully and objectively quantified several different facets of hypersomnolence, including ad libitum polysomnography to measure total sleep time.

There are several lines of evidence that further support the role of PAX8/PAX8-AS1, and particularly its epigenetic modification, on sleep duration and hypersomnia. First, one of the few compounds that has demonstrated a measurable impact on excessive sleep duration in idiopathic hypersomnia is levothyroxine, evaluated in a small open-label study in euthyroid patients.⁴⁰ While PAX8 is expressed in many tissues, it is predominantly expressed in fetal and adult thyroid tissue, and mutations in PAX8 have been associated with thyroid dysgenesis and thyroid cancers. Second, fluctuations in cerebral PAX8 methylation levels mirror patterns of total sleep quantity across the lifespan, with a high rate of change early in the first few years of life, followed by a general plateau beginning in the second decade.⁴¹ Thus, it is probable given the large number of GWAS studies implicating PAX8/PAX8-AS1 in sleep duration, relevant supportive data in the literature, and our findings linking PAX8/PAX8-AS1 methylation to sleep duration, that DNA methylation changes in PAX8/PAX8-AS1 may be a biomarker for, or mechanistically related to hypersomnia.

In the secondary genome-wide analysis, DMPs also were found in two genes that have been associated with hypersomnolence in other investigations, LHX6 and GABRA2. LHX6-positive GABA-releasing neurons in the zona incerta promote NREM sleep, likely by inhibiting hypocretin positive neurons, consistent with a plausible connection between these findings and sleep propensity quantified by the MSLT.⁴⁷ A SNP in GABRA2 was recently associated with self-reported daytime sleepiness in a large-scale GWAS study, published after the study methods were executed.⁴⁸ Together, these data suggest that genes other than PAX8 may exhibit disruptions in DNA methylation levels correlated to other aspects of hypersomnolence. Thus, future studies that both replicate the primary finding of an association between PAX8/PAX8-AS1 methylation and sleep duration, as well as associations between LHX6 and MSLT sleep latency and GABRA2 and infrared pupillometry, are indicated.

In summary, the present examples demonstrate a significant association between PAX8/PAX8-AS1 DNA methylation levels and objectively quantified sleep duration in persons with unexplained hypersomnolence. These results represent a key step forward in understanding the underlying biology that may be related to both sleep duration and pathological hypersomnia, bridging key findings in prior GWAS studies to disordered populations in sleep medicine. This work will serve as a nidus for further investigation that clarifies the role of PAX8/PAX8-AS1 regulation and expression in both sleep need and duration for healthy persons and patients with pathological hypersomnia.

### References

1. Dauvilliers Y, Arnulf I, Mignot E. Narcolepsy with cataplexy. Lancet. 2007; 369 (9560): 499-511.
2. Bliwise D L. Is the measurement of sleepiness the Holy Grail of sleep medicine? Am J Respir Crit Care Med. 2001; 163 (7): 1517-1519.
3. Pizza F. Sleepiness Assessment. In: Sleepiness and Human Impact Assessment. Milano, Italy.: Springer; 2014: 313-324.
4. Lopez R, Doukkali A, Barateau L, et al. Test-Retest Reliability of the Multiple Sleep Latency Test in Central Disorders of Hypersomnolence. Sleep. 2017; 40 (12).
5. Ruoff C, Pizza F, Trotti L M, et al. The MSLT is Repeatable in Narcolepsy Type 1 But Not Narcolepsy Type 2: A Retrospective Patient Study. J Clin Sleep Med. 2017.
6. Trotti L M, Staab B A, Rye D B. Test-retest reliability of the multiple sleep latency test in narcolepsy without cataplexy and idiopathic hypersomnia. J Clin Sleep Med. 2013; 9 (8): 789-795.
7. Sangal R B, Thomas L, Mitler M M. Maintenance of wakefulness test and multiple sleep latency test. Measurement of different abilities in patients with sleep disorders. Chest. 1992; 101 (4): 898-902.
8. Johns M W. Sensitivity and specificity of the multiple sleep latency test (MSLT), the maintenance of wakefulness test and the epworth sleepiness scale: failure of the MSLT as a gold standard. J Sleep Res. 2000; 9 (1): 5-11.
9. Plante D T, Cook J D, Prairie M L. Multimodal Assessment Increases Objective Identification of Hypersomnolence in Patients Referred for Multiple Sleep Latency Testing. J Clin Sleep Med. 2020.
10. Robinson P N. Deep phenotyping for precision medicine. Hum Mutat. 2012; 33 (5): 777-780.
11. Raizen D M, Wu M N. Genome-wide association studies of sleep disorders. Chest.
12. Gottlieb D J, Hek K, Chen T H, et al. Novel loci associated with usual sleep duration: the CHARGE Consortium Genome-Wide Association Study. Mol Psychiatry. 2015; 20 (10): 1232-1239.
13. Lane J M, Liang J, Vlasac I, et al. Genome-wide association analyses of sleep disturbance traits identify new loci and highlight shared genetics with neuropsychiatric and metabolic traits. Nat Genet. 2017; 49 (2): 274-281.
14. Dashti H S, Jones S E, Wood A R, et al. Genome-wide association study identifies genetic loci for self-reported habitual sleep duration supported by accelerometer-derived estimates. Nat Commun. 2019; 10 (1): 1100.
15. Jones S E, Tyrrell J, Wood A R, et al. Genome-Wide Association Analyses in 128,266 Individuals Identifies New Morningness and Sleep Duration Loci. PLoS Genet. 2016; 12 (8): e1006125.
16. Jones S E, van Hees V T, Mazzotti D R, et al. Genetic studies of accelerometer-based sleep measures yield new insights into human sleep behaviour. Nat Commun. 2019; 10 (1): 1585.
17. Littner MR, Kushida C, Wise M, et al. Practice parameters for clinical use of the multiple sleep latency test and the maintenance of wakefulness test. Sleep. 2005; 28 (1): 113-121.
18. Berry R, Brooks R, Gamaldo C, et al. The AASM Manual for the Scoring of Sleep and Associated Events: Rules, Terminology and Technical Specifications, Version 2.0. Darien, Ill.: American Academy of Sleep Medicine; 2012.
19. Eggert T, Sauter C, Popp R, Zeitlhofer J, Danker-Hopfe H, et al. The Pupillographic SleepinessTest in adults: effect of age, gender, and time of day on pupillometric variables. Am J Hum Biol. 2012; 24 (6): 820-828.
20. Lüdtke H, Wilhelm B, Adler M, Schaeffel F, Wilhelm H. Mathematical procedures in data recording and processing of pupillary fatigue waves. Vision Res. 1998; 38 (19): 2889-2896.
21. Wilhelm B, Korner A, Heldmaier K, Moll K, Wilhelm H, Ludtke H. Normative Values of the Pupillographic Sleepiness Test in Male and Female Subjects Aged 20 to 60 Years. Somnologie. 2001; 5: 115-120.
22. Ludtke H, Korner A, Wilhelm B, Wilhelm H. Reproducibility of the pupillographic sleepiness test in healthy men. Somnologie. 2000; 4: 170-172.
23. Wilhelm B, Bittner E, Hofmann A, et al. Short-term reproducibility and variability of the pupillographic sleepiness test. Am J Hum Biol. 2015; 27 (6): 862-866.
24. Dinges D F, Powell J W. Microcomputer analysis of performance on a portable, simple visual RT task sustained operations. Behavioral Research Methods, Instrumentation, and Computers. 1985; 17: 652-655.
25. Aryee M J, Jaffe A E, Corrada-Bravo H, et al. Minfi: a flexible and comprehensive Bioconductor package for the analysis of Infinium DNA methylation microarrays. Bioinformatics (Oxford, England). 2014; 30 (10): 1363-1369.
26. Maksimovic J, Gordon L, Oshlack A. SWAN: Subset-quantile within array normalization for illumina infinium HumanMethylation450 BeadChips. Genome biology. 2012; 13 (6): R44.
27. Wockner L F, Noble E P, Lawford B R, et al. Genome-wide DNA methylation analysis of human brain tissue from schizophrenia patients. Transl Psychiatry. 2014; 4: e339.
28. Johns M W. A new method for measuring daytime sleepiness: the Epworth sleepiness scale. Sleep. 1991; 14 (6): 540-545.
29. Leek J T, Johnson W E, Parker H S, Jaffe A E, Storey J D. The sva package for removing batch effects and other unwanted variation in high-throughput experiments. Bioinformatics (Oxford, England). 2012; 28 (6): 882-883.
30. Ritchie M E, Phipson B, Wu D, et al. limma powers differential expression analyses for RNA-sequencing and microarray studies. Nucleic acids research. 2015; 43 (7): e47.
31. Kuan P F, Chiang D Y. Integrating prior knowledge in multiple testing under dependence with applications to detecting differential DNA methylation. Biometrics. 2012; 68 (3): 774-783.
32. Ohayon M M, Reynolds C F, Dauvilliers Y. Excessive sleep duration and quality of life. Ann Neurol. 2013; 73 (6): 785-794.
33. Lammers G J, Bassetti CLA, Dolenc-Groselj L, et al. Diagnosis of central disorders of hypersomnolence: A reappraisal by European experts. Sleep Med Rev. 2020; 52: 101306.
34. Pelechano V, Steinmetz L M. Gene regulation by antisense transcription. Nat Rev Genet. 2013; 14 (12): 880-893.
35. Katayama S, Tomaru Y, Kasukawa T, et al. Antisense transcription in the mammalian transcriptome. Science. 2005; 309 (5740): 1564-1566.
36. Hongay C F, Grisafi P L, Galitski T, Fink G R. Antisense transcription controls cell fate in Saccharomyces cerevisiae. Cell. 2006; 127 (4): 735-745.
37. Wang Z, Wilson C M, Mendelev N, et al. Acute and Chronic Molecular Signatures and Associated Symptoms of Blast Exposure in Military Breachers. J Neurotrauma. 2020; 37 (10): 1221-1232.
38. Fernandez L P, Lopez-Marquez A, Santisteban P. Thyroid transcription factors in development, differentiation and disease. Nat Rev Endocrinol. 2015; 11 (1): 29-42.
39. Leenen F A, Muller C P, Turner J D. DNA methylation: conducting the orchestra from exposure to phenotype? Clin Epigenetics. 2016; 8: 92.
40. Shinno H, Ishikawa I, Yamanaka M, et al. Effect of levothyroxine on prolonged nocturnal sleep time and excessive daytime somnolence in patients with idiopathic hypersomnia. Sleep Med. 2011; 12 (6): 578-583.
41. Siegmund K D, Connor C M, Campan M, et al. DNA methylation in the human cerebral cortex is dynamically regulated throughout the life span and involves differentiated neurons. PLoS One. 2007; 2 (9): e895.
42. Trivedi M S, Holger D, Bui A T, Craddock T J A, Tartar J L. Short-term sleep deprivation leads to decreased systemic redox metabolites and altered epigenetic status. PLoS One. 2017; 12 (7): e0181978.
43. Nilsson E K, Bostrom A E, Mwinyi J, Schioth H B. Epigenomics of Total Acute Sleep Deprivation in Relation to Genome-Wide DNA Methylation Profiles and RNA Expression. OMICS. 2016; 20 (6): 334-342.
44. Vernet C, Arnulf I. Idiopathic hypersomnia with and without long sleep time: a controlled series of 75 patients. Sleep. 2009; 32 (6): 753-759.
45. Evangelista E, Lopez R, Barateau L, et al. Alternative diagnostic criteria for idiopathic hypersomnia: A 32-hour protocol. Ann Neurol. 2018; 83 (2): 235-247.
46. Pizza F, Moghadam K K, Vandi S, et al. Daytime continuous polysomnography predicts MSLT results in hypersomnias of central origin. J Sleep Res. 2013; 22 (1): 32-40.
47. Liu K, Kim J, Kim D W, et al. Lhx6-positive GABA-releasing neurons of the zona incerta promote sleep. Nature. 2017; 548 (7669): 582-587.
48. Wang H, Lane J M, Jones S E, et al. Genome-wide association analysis of self-reported daytime sleepiness identifies 42 loci that suggest biological subtypes. Nat Commun. 2019; 10 (1): 3503.
49. Cortese R, Zhang C, Bao R, et al. DNA Methylation Profiling of Blood Monocytes in Patients With Obesity Hypoventilation Syndrome: Effect of Positive Airway Pressure Treatment. Chest. 2016; 150 (1): 91-101.
50. Ammala A J, Urrila A S, Lahtinen A, et al. Epigenetic dysregulation of genes related to synaptic long-term depression among adolescents with depressive disorder and sleep symptoms. Sleep Med. 2019; 61: 95-103.
51. Alisch R S, Chopra P, Fox A S, et al. Differentially methylated plasticity genes in the amygdala of young primates are linked to anxious temperament, an at risk phenotype for anxiety and depressive disorders. J Neurosci. 2014; 34 (47): 15548-15556.
52. Papale L A, Seltzer L J, Madrid A, Pollak S D, Alisch R S. Differentially Methylated Genes in Saliva are linked to Childhood Stress. Sci Rep. 2018; 8 (1): 10785.
53. Alisch R S, Van Hulle C, Chopra P, et al. A multi-dimensional characterization of anxiety in monozygotic twin pairs reveals susceptibility loci in humans. Transl Psychiatry. 2017; 7 (12): 1282.
54. Kim H, Dinges DF, Young T. Sleep-disordered breathing and psychomotor vigilance in a community-based sample. Sleep. 2007; 30 (10): 1309-1316.
55. Jansen E C, Dolinoy D C, O'Brien L M, et al. Sleep duration and fragmentation in relation to leukocyte DNA methylation in adolescents. Sleep. 2019; 42 (9).
56. Leu-Semenescu S, Louis P, Arnulf I. Benefits and risk of sodium oxybate in idiopathic hypersomnia versus narcolepsy type 1: a chart review. Sleep Med. 2016; 17: 38-44.
57. Gottlieb D J, O'Connor G T, Wilk J B. Genome-wide association of sleep and circadian phenotypes. BMC Med Genet. 2007; 8 Suppl 1: S9.
58. Pak V M, Mazzotti D R, Keenan B T, et al. Candidate Gene Analysis in the Sao Paulo Epidemiologic Sleep Study (EPISONO) Shows an Association of Variant in PDE4D and Sleepiness. Sleep Med. 20181 47: 106-12.
59. Chen Y C, Chen T W, Su M C, et al. Whole Genome DNA Methylation Analysis of Obstructive Sleep Apnea: IL1R2, NPR2, AR, SP140 Methylation and Clinical Phenotype. Sleep. 2016; 39 (4): 743-755.
60. Fang J, Wang Y, Krueger J M. Effects of interleukin-1 beta on sleep are mediated by the type I receptor. Am J Physiol. 1998; 274 (3 Pt 2): R655-660.
61. Bodenmann S, Hohoff C, Freitag C, et al. Polymorphisms of ADORA2A modulate psychomotor vigilance and the effects of caffeine on neurobehavioural performance and sleep EEG after sleep deprivation. Br J Pharmacol. 2012; 165 (6): 1904-1913.
62. Rupp T L, Wesensten N J, Newman R, Balkin T J. PER3 and ADORA2A polymorphisms impact neurobehavioral performance during sleep restriction. J Sleep Res. 2013; 22 (2): 160-165.
63. Goel N, Banks S, Mignot E, Dinges D F. PER3 polymorphism predicts cumulative sleep homeostatic but not neurobehavioral changes to chronic partial sleep deprivation. PLoS One. 2009; 4 (6): e5874.
64. Shimada M, Miyagawa T, Kawashima M, et al. An approach based on a genome-wide association study reveals candidate loci for narcolepsy. Hum Genet. 2010; 128 (4): 433-441.
65. Byrne E M, Gehrman P R, Medland S E, et al. A genome-wide association study of sleep habits and insomnia. Am J Med Genet B Neuropsychiatr Genet. 2013; 162B (5): 439-451.
66. Parsons M J, Lester K J, Barclay N L, Nolan P M, Eley T C, Gregory A M. Replication of Genome-Wide Association Studies (GWAS) loci for sleep in the British G1219 cohort. Am J Med Genet B Neuropsychiatr Genet. 2013; 162B (5): 431-438.
67. Huang H, Zhu Y, Eliot MN, et al. Combining Human Epigenetics and Sleep Studies in Caenorhabditis elegans: A Cross-Species Approach for Finding Conserved Genes Regulating Sleep. Sleep. 2017; 40 (6).

1. A method comprising:
isolating target DNA from a biofluid sample from a subject to obtain isolated target DNA;
treating the isolated target DNA with bisulfite to generate bisulfite modified DNA;
amplifying the bisulfite modified DNA with primers specific to one or more human genomic positions to generate amplified DNA, wherein the one or more human genomic positions comprise one or more of 113992930 of chromosome 2, 113993052 of chromosome 2, 113993142 of chromosome 2, 113993304 of chromosome 2, 113993313 of chromosome 2, 113994035 of chromosome 2, 113994578 of chromosome 2, 113995370 of chromosome 2, 113995456 of chromosome 2, 124985406 of chromosome 9, 124985756 of chromosome 9, 124987896 of chromosome 9, 124988720 of chromosome 9, 124989052 of chromosome 9, 124989241 of chromosome 9, 124989294 of chromosome 9, 124989337 of chromosome 9, 124989408 of chromosome 9, 124989550 of chromosome 9, 124989839 of chromosome 9, 46391392 of chromosome 4, 46391436 of chromosome 4, 46391448 of chromosome 4, 46391476 of chromosome 4, 46391524 of chromosome 4, 46391532 of chromosome 4, and 46391694 of chromosome 4; and
quantifying methylation at the one or more human genomic positions in the amplified DNA.

2. The method of claim 1, wherein the one or more human genomic positions comprise one or more of 113993142 of chromosome 2, 113993304 of chromosome 2, 113993313 of chromosome 2, 113994578 of chromosome 2, 113995370 of chromosome 2, and 113995456 of chromosome 2.

3. The method of claim 1, wherein the one or more human genomic positions comprise one or more of 113994578 of chromosome 2, 113995370 of chromosome 2, and 113995456 of chromosome 2.

4. The method of claim 1, wherein the one or more human genomic positions comprise seven or more of 113992930 of chromosome 2, 113993052 of chromosome 2, 113993142 of chromosome 2, 113993304 of chromosome 2, 113993313 of chromosome 2, 113994035 of chromosome 2, 113994578 of chromosome 2, 113995370 of chromosome 2, and 113995456 of chromosome 2.

5. The method of claim 1, wherein the one or more human genomic positions comprise each of 113992930 of chromosome 2, 113993052 of chromosome 2, 113993142 of chromosome 2, 113993304 of chromosome 2, 113993313 of chromosome 2, 113994035 of chromosome 2, 113994578 of chromosome 2, 113995370 of chromosome 2, and 113995456 of chromosome 2.

6. The method of claim 1, wherein the one or more human genomic positions comprise one or more of 124985406 of chromosome 9, 124985756 of chromosome 9, 124987896 of chromosome 9, 124988720 of chromosome 9, 124989052 of chromosome 9, 124989241 of chromosome 9, 124989294 of chromosome 9, 124989337 of chromosome 9, 124989408 of chromosome 9, 124989550 of chromosome 9, 124989839 of chromosome 9, 46391392 of chromosome 4, 46391436 of chromosome 4, 46391448 of chromosome 4, 46391476 of chromosome 4, 46391524 of chromosome 4, 46391532 of chromosome 4, and 46391694 of chromosome 4.

7. The method of claim 1, wherein the one or more human genomic positions comprise one or more of 124985406 of chromosome 9, 124985756 of chromosome 9, 124987896 of chromosome 9, 124988720 of chromosome 9, 124989052 of chromosome 9, 124989241 of chromosome 9, 124989294 of chromosome 9, 124989337 of chromosome 9, 124989408 of chromosome 9, 124989550 of chromosome 9, and 124989839 of chromosome 9.

8. The method of claim 1, wherein the one or more human genomic positions comprise each of 124985406 of chromosome 9, 124985756 of chromosome 9, 124987896 of chromosome 9, 124988720 of chromosome 9, 124989052 of chromosome 9, 124989241 of chromosome 9, 124989294 of chromosome 9, 124989337 of chromosome 9, 124989408 of chromosome 9, 124989550 of chromosome 9, and 124989839 of chromosome 9.

9. The method of claim 1, wherein the one or more human genomic positions comprise one or more of 46391392 of chromosome 4, 46391436 of chromosome 4, 46391448 of chromosome 4, 46391476 of chromosome 4, 46391524 of chromosome 4, 46391532 of chromosome 4, and 46391694 of chromosome 4.

10. The method of claim 1, wherein the one or more human genomic positions comprise each of 46391392 of chromosome 4, 46391436 of chromosome 4, 46391448 of chromosome 4, 46391476 of chromosome 4, 46391524 of chromosome 4, 46391532 of chromosome 4, and 46391694 of chromosome 4.

11-26. (canceled)

27. A method of amplification, the method comprising:
providing a reaction mixture comprising bisulfite modified target DNA from a biofluid sample from a subj ect and primers specific to one or more human genomic positions, wherein the one or more human genomic positions comprise one or more of 113992930 of chromosome 2, 113993052 of chromosome 2, 113993142 of chromosome 2, 113993304 of chromosome 2, 113993313 of chromosome 2, 113994035 of chromosome 2, 113994578 of chromosome 2, 113995370 of chromosome 2, 113995456 of chromosome 2, 124985406 of chromosome 9, 124985756 of chromosome 9, 124987896 of chromosome 9, 124988720 of chromosome 9, 124989052 of chromosome 9, 124989241 of chromosome 9, 124989294 of chromosome 9, 124989337 of chromosome 9, 124989408 of chromosome 9, 124989550 of chromosome 9, 124989839 of chromosome 9, 46391392 of chromosome 4, 46391436 of chromosome 4, 46391448 of chromosome 4, 46391476 of chromosome 4, 46391524 of chromosome 4, 46391532 of chromosome 4, and 46391694 of chromosome 4;
heating the reaction mixture to a first predetermined temperature for a first predetermined time;
cooling the reaction mixture to a second predetermined temperature for a second predetermined time under conditions to allow the primers to hybridize with complementary sequences on the bisulfite modified target DNA; and
repeating the heating and the cooling, wherein an amplified target DNA sample is formed.

28. The method of claim 27, wherein the one or more human genomic positions comprise one or more of 113993142 of chromosome 2, 113993304 of chromosome 2, 113993313 of chromosome 2, 113994578 of chromosome 2, 113995370 of chromosome 2, and 113995456 of chromosome 2.

29. The method of claim 27, wherein the one or more human genomic positions comprise one or more of 113994578 of chromosome 2, 113995370 of chromosome 2, and 113995456 of chromosome 2.

30. The method of claim 27, wherein the one or more human genomic positions comprise seven or more of 113992930 of chromosome 2, 113993052 of chromosome 2, 113993142 of chromosome 2, 113993304 of chromosome 2, 113993313 of chromosome 2, 113994035 of chromosome 2, 113994578 of chromosome 2, 113995370 of chromosome 2, and 113995456 of chromosome 2.

31. The method of claim 27, wherein the one or more human genomic positions comprise each of 113992930 of chromosome 2, 113993052 of chromosome 2, 113993142 of chromosome 2, 113993304 of chromosome 2, 113993313 of chromosome 2, 113994035 of chromosome 2, 113994578 of chromosome 2, 113995370 of chromosome 2, and 113995456 of chromosome 2.

32. The method of claim 27, wherein the one or more human genomic positions comprise one or more of 124985406 of chromosome 9, 124985756 of chromosome 9, 124987896 of chromosome 9, 124988720 of chromosome 9, 124989052 of chromosome 9, 124989241 of chromosome 9, 124989294 of chromosome 9, 124989337 of chromosome 9, 124989408 of chromosome 9, 124989550 of chromosome 9, 124989839 of chromosome 9, 46391392 of chromosome 4, 46391436 of chromosome 4, 46391448 of chromosome 4, 46391476 of chromosome 4, 46391524 of chromosome 4, 46391532 of chromosome 4, and 46391694 of chromosome 4.

33. The method of claim 27, wherein the one or more human genomic positions comprise one or more of 124985406 of chromosome 9, 124985756 of chromosome 9, 124987896 of chromosome 9, 124988720 of chromosome 9, 124989052 of chromosome 9, 124989241 of chromosome 9, 124989294 of chromosome 9, 124989337 of chromosome 9, 124989408 of chromosome 9, 124989550 of chromosome 9, and 124989839 of chromosome 9.

34. The method of claim 27, wherein the one or more human genomic positions comprise each of 124985406 of chromosome 9, 124985756 of chromosome 9, 124987896 of chromosome 9, 124988720 of chromosome 9, 124989052 of chromosome 9, 124989241 of chromosome 9, 124989294 of chromosome 9, 124989337 of chromosome 9, 124989408 of chromosome 9, 124989550 of chromosome 9, and 124989839 of chromosome 9.

35. The method of claim 27, wherein the one or more human genomic positions comprise one or more of 46391392 of chromosome 4, 46391436 of chromosome 4, 46391448 of chromosome 4, 46391476 of chromosome 4, 46391524 of chromosome 4, 46391532 of chromosome 4, and 46391694 of chromosome 4.

36. The method of claim 27, wherein the one or more human genomic positions comprise each of 46391392 of chromosome 4, 46391436 of chromosome 4, 46391448 of chromosome 4, 46391476 of chromosome 4, 46391524 of chromosome 4, 46391532 of chromosome 4, and 46391694 of chromosome 4.

37-44. (canceled)

45. A biomarker panel comprising a probe, primers, or a combination thereof specific to one or more human genomic positions, wherein the one or more human genomic positions comprise one or more of 113992930 of chromosome 2, 113993052 of chromosome 2, 113993142 of chromosome 2, 113993304 of chromosome 2, 113993313 of chromosome 2, 113994035 of chromosome 2, 113994578 of chromosome 2, 113995370 of chromosome 2, 113995456 of chromosome 2, 124985406 of chromosome 9, 124985756 of chromosome 9, 124987896 of chromosome 9, 124988720 of chromosome 9, 124989052 of chromosome 9, 124989241 of chromosome 9, 124989294 of chromosome 9, 124989337 of chromosome 9, 124989408 of chromosome 9, 124989550 of chromosome 9, 124989839 of chromosome 9, 46391392 of chromosome 4, 46391436 of chromosome 4, 46391448 of chromosome 4, 46391476 of chromosome 4, 46391524 of chromosome 4, 46391532 of chromosome 4, and 46391694 of chromosome 4.

46-65. (canceled)


SEQUENCE LISTING (37 sequences)

SEQ ID NO: 1 - DNA, 385 bp, Homo sapiens
cgccgccatagctgcatggccccgggacctccctgtcgtacctgagaggagggcctggcccgtgaactgcccgtacacggaggcagcatggggaaaggcattgaagggcgggaccccggagccgacttgctgcagatccaaaaaggcggagctagataaagaggaaggggtggagctagaactggacacctcgggggtttcctgctttatggcgaagggtgagtgaggatctgccggagggagggagacaacaaggagagaggggtgtgagatggcggggagggaacacgcacaagccaagccgtgggatgtggagggtgcgggcggggcgcggggcaggctcagctgccctcagagtctgggctggggagagccggggcccacg

SEQ ID NO: 2 - DNA, 2528 bp, Homo sapiens
cgccgccatagctgcatggccccgggacctccctgtcgtacctgagaggagggcctggcccgtgaactgcccgtacacggaggcagcatggggaaaggcattgaagggcgggaccccggagccgacttgctgcagatccaaaaaggcggagctagataaagaggaaggggtggagctagaactggacacctcgggggtttcctgctttatggcgaagggtgagtgaggatctgccggagggagggagacaacaaggagagaggggtgtgagatggcggggagggaacacgcacaagccaagccgtgggatgtggagggtgcgggcggggcgcggggcaggctcagctgccctcagagtctgggctggggagagccggggcccacgaggcgtggcaaggcggggagagagaagctagacctccctgctgcgcttctgcagcgaaaatggagagacccggaaggcgtgtgtgcgcccgcctctcccacaggagggagcgcggagacccgggagcggcctaggaccggaggcgcgacccctcggcccaccttgaggcccggcctaggaccggaggcgcgacccctgggcccaccttgaggcccggcctaggactggaggcgcgacccctcggcccaccttgcgggagccgcctaggaccggaggcgcgacccctcggcccagcttgaggcccggcctaggaccggaggcgcgacccctcggcccaccttgcgggagccgcctaggaccggaggcgcgacccctcggcccaccttgaggcccggcctaggaccggaggcgcgacccctgggcccaccttgaggcccggcctaggactggaggcgcgacccctcggcccaccttacgggagccgcctagggccggaggcgcgacccctcggcccagcttgaggcccggcctaggaccggaggcgcgacccctcggcccaccttgaggcccggcctaggactggaggcgcgacccctcggcccaccttgaggcccggcctaggaccggaggcgcgacccctgggcccaccttgaggcccggcctaggactggaggcgcgacccctcggcccaccttgaggcccggcctaggaccggaggcgcgacccctgggcccacctggcggcccggccggcacagcccgcctctcctctccaggccagggccccacaccttccgcctgacagccagccaagctcttcagtcccccgccctccacctgccagggaggctccgggcgttgtacctgccaccacggggtaggtctggtgagtcgagaggttgcgccccagtggcgtgttggaaggggtcagggtggccttcccgtcgtccagggtgctgttgagcaagggcagcgggtagaggccctggggagcaaagagaagtcagcgcacagagacgatccaacaagccgaccttcctgcagaccctgagcctgtgtcttaggacttggcagccctcatctccccaggagaggtcctcatcgcccccttcttcctttacgttctcaactccactcggcacgtttcgttcatgctcattgtcctcccgcatccctggaattcaaggccagctggtagcatgggtgcccagacgtgggatagagagtctcagccagaggaccacaccctggttggatggctggtcggcttcctggtttcaccacactgttgggacccacagaatgcaggacggtgccaaggactggagcagtctccaaccggactgttcagggtccttgcatctgttaatttattcttccccagataggaaacccaggcacaaaggtttaagaaacttgtcccacttccagatgggccaaagataggattcagattcgagggatcagaatgtgtctgctctcaaccactgccctaaactgggtgacataaggaccacttcttacatttgcaaagagcttatatcagagctttgcctctgttgtcctagtagagtcccccagcagcccagtgacatgggtgagtaatattatgtctgtgttttatatgtggagcctggaactcaggtaaatggcccaattttgtatagtgaaacagactaaacccgggatttttcggctgtggacccaggtgctttacccaccacgtggatttcttcttttctctctccacatccaaacaaatagaagtgtgcaagttcggcttgaaaatggtattgagagttgtttttgttggaaacactctttgtcaagtacctgggggtcttcatcagtaagacctcattttagatgcctcctggcctccccttcccaggagcacagctatgaccttaggtactccttccgaaaagaacttgtttaactaaaggtaagtgtacctcatcctcaccatggcctccttccactggggaagcagatagcgcagaaaaaagaacacacccattccccacataccttcacactcgtcacatacctgctacgtgagatgtgcaaagctgaattcagggaatgctcagtagttacataacagtgccactaaaggcaattgttttcagtgatttccatcg

SEQ ID NO: 3 - DNA, 4435 bp, Homo sapiens
cgatcggctttttctttccctttttgtgggttttattatttttgttacaggtggggctgccagggatgggttatgcctgcagggggagaaatgatcagaagagccacaattctaccctcaacccagggaatgtgagcttttactgcctccattttcttaaccagaaatctggtttagttttgctttcagggctcttgtcattggaaaaagattagtgctcagaacctgcagcccagaaatggcccagtcccatccctccaggcaggctctgctcctcaagctgcgtggggaaatgccatcatgggcttgtgcccagcatccaccctgccaatgatgacaacagtgataatcgaggcattcataccaaccaccatttagggagcctttatagagacttccccccaacagtggcctctttggcagctctgggagacagccttggaaagggcgaactaagggccgagagaagcagcaacttgcgaaggtcactactggccagactgggcccagtctcctgcctcttcccacagtggctgcacaccctactctctaccccattgcagtaggaagagaactccagaataaggggaggctctgaacagtggagtcaggatccgtgttttgactagtttcaaggtctagtaaagcacagtatccaaggaggctcagtccagcatgggcatgggctagggctggagaagtagaaaagaaatttgggcaagtagtgaataagactgccaaatctattgttaggagagccaggagaggtgtcctagtggtgccttagtctatgggagagacccagacaccattctgctgggttcccaagctggcaccttaaatttagtagctgatgaatccaaggagcccccaagcatcatctggcatgtttttttgtatgtttgtttgtttgttttttgagacagagtctctctctgttacccaggctggagtgtagtgacgtgatcttggctcaccacaatgtccgcctcccaggttcaagtgattctcttgcctcagcctcctgagtagctgggactacaggcacgtgccaccacacctggctaagttttgtatttttagtagagatgggtttcaccatgttggccaggatggtctcgatctcctgaccttgtgatctaccagccttggcctcccaaagtgctgcgattataggtgtgagccaccacacccggcccatctggcatgttttataccaactggagtcaggcctagagcagggaggtggctagacagtgactgtaacctccatttcctgtcctggccaaaccaatactcagatgggacagtgagaggagacatcacactctggttacacaccattaacccctgtctgactacacacacacacacacacagccgtccagattcacaaatacgttcttatcacccccttcatccctcagtgcctgggtggaggcccttaagcaggaatgccccctcagaggagagggtgggagtggaatgatgggatttcagaatcccctcacctcaccctctcacctccagatcctagtccagatgtaatcctgtcctcaccactttgcgtgcacgtgcacgcacacacacatgcacacacacagtctcaacattcacatacagacacccactcttgacacccacacatgaactaaaagacaaacgttggtccataaaggaacacacatgctatctacatacccccaaccctgcccagtgatgctttccccagcactactgcacagatcccagacaatacagacacacaggcactccttgcacactgagtcctaggccacttggttgagctgcccccaccccaccccagctgcacactcttctacatccatcccataaaggaagagtccgtctctttaaccatgcttctcatggttttcagagtacccttgccccaaatgctgccaacctccccctagactcaagatcttctctttccagatcgctggaggctcaatgccagtgcacaggaggagatcgccttccaccatattccctgactcccagaggctctttctagggcaggggtctggccaggaagactgaagccatgacgtcttcctgtgcaaggataggaaagatgtctgtgggttggagggtagttatagggaagcagctttctccactttttgagaaagcacttgggaacaagcatccctctcaggaagtgattctccaccattctcctctgatcctataggcctggtggtccaagcctcaggccagttgatgcctcccactgggatcagcagacaaggccatgaccctgcagtgacctcgaccccataatcaggtcaggtccgggcttcatggctagaagcgaggcccaggcgagtggccaagacgcggcaggacaggaggatgcgcctggctgctctcggctgctggacgcggcctgcagtagggagagcgcagcccggggtaatcccactcagggctccgggtgcccagttctcccacttcccatcctggcggcctaaaggcaaggcctagaccctggagaggccaaagtcgggccaaaacaacagcgaagctgggactggaacaggcgctgggggcctgcgcatgaccccaggctcagaatgcgacactccagtgacgccggggaatgagttgagatggagccggagacagaggttggggttcaggacgaggcgcttgggtatgggatcggatctgagacggttgcggcgctggtgctttgggctggaccagactcgaacccagagcggagcgagggtcagagccagggcaagagtccgaagcccagctcagcgtccagcccgcgagagaccccaccatgaatgacgtccgagtagggttggaactcgaagcccgaacgcactggaaatgcagccggacgacggctgggatccgagtgggtccgggccagaaatggggacctcagagctccaccgaggcgctcgaggtcagaactgaagcctgagacttaggcaggacccgagacagagccagagacgataccgaaacccaatggaccgcgaggaccgaaggcagatccggggcgcaaacctgtgcaggcactggcgcgctgcctggagctctgggttcgcgccgctgagcgccggcaggttggaccagcgctgcgcgccggaagtgcactcgggacgccggggttctggaggagagaccacacgccgcaaaagtggcctccgaatgcgcccggggcctgccctcggcgacactgctggctcggcggccgcagcctacctgaagtagtccatcttgcagaagatctccttgttcttgatgtagcagctgttctgctgcctcagcgacgtgcgacacacggagcactcgaggcaccgcacgtgccagatgaggttgttgacctggggacggggcggggacggaggtcggctcagcgcgggcctctttcactccgggccccagccttcccggtctcatttacagtcagaggcgctgaagctcagaaggtgcatgcgattcccctatttttctcccgtaatactgagactcagggaaatggaaataaactcttcttatttttcagacggaacccgggggctcaggcagaccctaagtcttgcccaaagcttcgcagtcgggcagcagtggagccaggatttggaagtaagaggacctgcggtgcttccctgcagtctcctgcgctgcgtcccacgccccgacaacacgcacgcaacacctacccctgtctcaccttgagcagatatcggtccaggatctcgaggccgcagctggagcagatgttcttgcctgcagacggcacggaggaggcggcagagggcggtgagcagacagatggcgtgctgggcgtacatggggaggcctgaccctcgtccttgtccagagctcctgccagggcctcggcgtcagactgagcctgcggcgggggagagaaggagaggagcgctgatccgggcacccagagttggggctgccttggagacaaatctggggcccccgagcaccaattgacaacgaaatctccccaggacaatgcgccgtagactgaaggacagggatggaagggcctattggagaggaagggacggccccaacttttaacacgcgcattctgggtgctgggagcggttaaagccaacattccctgccggaaaaccgaggctcagagaggggcggcgcccgccgggagtcccccagagcgtgagcagcagttcgtggccggaaaacctggccaggtccccagggccgggccgctggaggactgggcaccgcaggcagggccaaacggactcaccatggcgggcggcgcggtcccttcaagacagcgggtggtcgctttgcagccggaccctggctgggccatcacctgggggagggggggagggaacgcaggcggcggcggctgctgaactggctccgcacagcctgagcccagcgcctccccgcg

SEQ ID NO: 4 - DNA, 500 bp, Homo sapiens
cggtgcttccctgcagtctcctgcgctgcgtcccacgccccgacaacacgcacgcaacacctacccctgtctcaccttgagcagatatcggtccaggatctcgaggccgcagctggagcagatgttcttgcctgcagacggcacggaggaggcggcagagggcggtgagcagacagatggcgtgctgggcgtacatggggaggcctgaccctcgtccttgtccagagctcctgccagggcctcggcgtcagactgagcctgcggcgggggagagaaggagaggagcgctgatccgggcacccagagttggggctgccttggagacaaatctggggcccccgagcaccaattgacaacgaaatctccccaggacaatgcgccgtagactgaaggacagggatggaagggcctattggagaggaagggacggccccaacttttaacacgcgcattctgggtgctgggagcggttaaagccaacattccctgccggaaaaccg

SEQ ID NO: 5 - DNA, 304 bp, Homo sapiens
cgtttgacagctgctctggagccgaggatcacaataagagaaggcgcgtgaggctccggcagcaaacaccagccaggcaggcagcggtaatcacagtgagcaaagttgaagactataaaagagccaggctaacgtgctgccgagcaggtgtcctggattttattgtagttgcaaatatgcttctggtgataagtatcgaaattattttttaagtgcgctggggaggagggaggtgcgggaattgagcctcctccgtttccagggagcctctgaaagtggggtggggggagatgaggcatgcacg

SEQ ID NO: 6 - DNA, 885 bp, Homo sapiens
aggccaagttgctacgttacagcacccggctcccggccttagggttcctgcttttccatggggtcttggggtggtgcatccccggcacccctacagcatccgccccttccgagcatgtcttccccgtcacagagaacttcatgttggcgcctccaaaagttgccggaggaattagggaacagggtgggggtggatgagactgaggccagagagggggctggcggtctgccctgaggacccccgtcccacccgccgccatagctgcatggccccgggacctccctgtcgtacctgagaggagggcctggcccgtgaactgcccgtacacggaggcagcatggggaaaggcattgaagggcgggaccccggagccgacttgctgcagatccaaaaaggcggagctagataaagaggaaggggtggagctagaactggacacctcgggggtttcctgctttatggcgaagggtgagtgaggatctgccggagggagggagacaacaaggagagaggggtgtgagatggcggggagggaacacgcacaagccaagccgtgggatgtggagggtgcgggcggggcgcggggcaggctcagctgccctcagagtctgggctggggagagccggggcccacgaggcgtggcaaggcggggagagagaagctagacctccctgctgcgcttctgcagcgaaaatggagagacccggaaggcgtgtgtgcgcccgcctctcccacaggagggagcgcggagacccgggagcggcctaggaccggaggcgcgacccctcggcccaccttgaggcccggcctaggaccggaggcgcgacccctgggcccaccttgaggcccggcctaggactggaggcgcgacccctcggcccacc

SEQ ID NO: 7 - DNA, 885 bp, Artificial Sequence
  Bisulfite modified sequence
aggttaagttgttacgttatagtattcggttttcggttttagggtttttgtttttttatggggttttggggtggtgtattttcggtatttttatagtattcgtttttttcgagtatgtttttttcgttatagagaattttatgttggcgtttttaaaagttgtcggaggaattagggaatagggtgggggtggatgagattgaggttagagagggggttggcggtttgttttgaggattttcgttttattcgtcgttatagttgtatggtttcgggatttttttgtcgtatttgagaggagggtttggttcgtgaattgttcgtatacggaggtagtatggggaaaggtattgaagggcgggatttcggagtcgatttgttgtagatttaaaaaggcggagttagataaagaggaaggggtggagttagaattggatatttcgggggttttttgttttatggcgaagggtgagtgaggatttgtcggagggagggagataataaggagagaggggtgtgagatggcggggagggaatacgtataagttaagtcgtgggatgtggagggtgcgggcggggcgcggggtaggtttagttgtttttagagtttgggttggggagagtcggggtttacgaggcgtggtaaggcggggagagagaagttagatttttttgttgcgtttttgtagcgaaaatggagagattcggaaggcgtgtgtgcgttcgttttttttataggagggagcgcggagattcgggagcggtttaggatcggaggcgcgatttttcggtttattttgaggttcggtttaggatcggaggcgcgatttttgggtttattttgaggttcggtttaggattggaggcgcgatttttcggtttatt

SEQ ID NO: 8 - DNA, 20 bp, Artificial Sequence
  Primer
gaggttagagagggggttgg

SEQ ID NO: 9 - DNA, 20 bp, Artificial Sequence
  Primer
cgctacaaaaacgcaacaaa

SEQ ID NO: 10 - DNA, 1922 bp, Homo sapiens
ccttacgggagccgcctagggccggaggcgcgacccctcggcccagcttgaggcccggcctaggaccggaggcgcgacccctcggcccaccttgaggcccggcctaggactggaggcgcgacccctcggcccaccttgaggcccggcctaggaccggaggcgcgacccctgggcccaccttgaggcccggcctaggactggaggcgcgacccctcggcccaccttgaggcccggcctaggaccggaggcgcgacccctgggcccacctggcggcccggccggcacagcccgcctctcctctccaggccagggccccacaccttccgcctgacagccagccaagctcttcagtcccccgccctccacctgccagggaggctccgggcgttgtacctgccaccacggggtaggtctggtgagtcgagaggttgcgccccagtggcgtgttggaaggggtcagggtggccttcccgtcgtccagggtgctgttgagcaagggcagcgggtagaggccctggggagcaaagagaagtcagcgcacagagacgatccaacaagccgaccttcctgcagaccctgagcctgtgtcttaggacttggcagccctcatctccccaggagaggtcctcatcgcccccttcttcctttacgttctcaactccactcggcacgtttcgttcatgctcattgtcctcccgcatccctggaattcaaggccagctggtagcatgggtgcccagacgtgggatagagagtctcagccagaggaccacaccctggttggatggctggtcggcttcctggtttcaccacactgttgggacccacagaatgcaggacggtgccaaggactggagcagtctccaaccggactgttcagggtccttgcatctgttaatttattcttccccagataggaaacccaggcacaaaggtttaagaaacttgtcccacttccagatgggccaaagataggattcagattcgagggatcagaatgtgtctgctctcaaccactgccctaaactgggtgacataaggaccacttcttacatttgcaaagagcttatatcagagctttgcctctgttgtcctagtagagtcccccagcagcccagtgacatgggtgagtaatattatgtctgtgttttatatgtggagcctggaactcaggtaaatggcccaattttgtatagtgaaacagactaaacccgggatttttcggctgtggacccaggtgctttacccaccacgtggatttcttcttttctctctccacatccaaacaaatagaagtgtgcaagttcggcttgaaaatggtattgagagttgtttttgttggaaacactctttgtcaagtacctgggggtcttcatcagtaagacctcattttagatgcctcctggcctccccttcccaggagcacagctatgaccttaggtactccttccgaaaagaacttgtttaactaaaggtaagtgtacctcatcctcaccatggcctccttccactggggaagcagatagcgcagaaaaaagaacacacccattccccacataccttcacactcgtcacatacctgctacgtgagatgtgcaaagctgaattcagggaatgctcagtagttacataacagtgccactaaaggcaattgttttcagtgatttccatcgagctgggttctgcaaagatccacagcactttccggttgcatgctgggcacttttggaagctgcagtcaattctggaggccaccagggcaccattagcacatagcagcaattattgactaaatggtgctctggttccatgccttccaagggggcccacttagaggcagggtggagttgcttagggcctttttttttttttttttttgtagacggagttttgctcttgttgcccaagctggagtgcaatgg

SEQ ID NO: 11 - DNA, 1922 bp, Artificial Sequence
  Bisulfite modified sequence
ttttacgggagtcgtttagggtcggaggcgcgatttttcggtttagtttgaggttcggtttaggatcggaggcgcgatttttcggtttattttgaggttcggtttaggattggaggcgcgatttttcggtttattttgaggttcggtttaggatcggaggcgcgatttttgggtttattttgaggttcggtttaggattggaggcgcgatttttcggtttattttgaggttcggtttaggatcggaggcgcgatttttgggtttatttggcggttcggtcggtatagttcgttttttttttttaggttagggttttatatttttcgtttgatagttagttaagttttttagtttttcgttttttatttgttagggaggtttcgggcgttgtatttgttattacggggtaggtttggtgagtcgagaggttgcgttttagtggcgtgttggaaggggttagggtggttttttcgtcgtttagggtgttgttgagtaagggtagcgggtagaggttttggggagtaaagagaagttagcgtatagagacgatttaataagtcgatttttttgtagattttgagtttgtgttttaggatttggtagtttttatttttttaggagaggtttttatcgtttttttttttttttacgtttttaattttattcggtacgtttcgtttatgtttattgttttttcgtatttttggaatttaaggttagttggtagtatgggtgtttagacgtgggatagagagttttagttagaggattatattttggttggatggttggtcggttttttggttttattatattgttgggatttatagaatgtaggacggtgttaaggattggagtagtttttaatcggattgtttagggtttttgtatttgttaatttatttttttttagataggaaatttaggtataaaggtttaagaaatttgttttatttttagatgggttaaagataggatttagattcgagggattagaatgtgtttgtttttaattattgttttaaattgggtgatataaggattattttttatatttgtaaagagtttatattagagttttgtttttgttgttttagtagagttttttagtagtttagtgatatgggtgagtaatattatgtttgtgttttatatgtggagtttggaatttaggtaaatggtttaattttgtatagtgaaatagattaaattcgggatttttcggttgtggatttaggtgttttatttattacgtggatttttttttttttttttttatatttaaataaatagaagtgtgtaagttcggtttgaaaatggtattgagagttgtttttgttggaaatattttttgttaagtatttgggggtttttattagtaagattttattttagatgttttttggttttttttttttaggagtatagttatgattttaggtatttttttcgaaaagaatttgtttaattaaaggtaagtgtattttatttttattatggtttttttttattggggaagtagatagcgtagaaaaaagaatatatttattttttatatatttttatattcgttatatatttgttacgtgagatgtgtaaagttgaatttagggaatgtttagtagttatataatagtgttattaaaggtaattgtttttagtgatttttatcgagttgggttttgtaaagatttatagtatttttcggttgtatgttgggtatttttggaagttgtagttaattttggaggttattagggtattattagtatatagtagtaattattgattaaatggtgttttggttttatgttttttaagggggtttatttagaggtagggtggagttgtttagggtttttttttttttttttttttgtagacggagttttgtttttgttgtttaagttggagtgtaatgg

SEQ ID NO: 12 - DNA, 18 bp, Artificial Sequence
  Primer
tacgggagtcgtttaggg

SEQ ID NO: 13 - DNA, 18 bp, Artificial Sequence
  Primer
ctcaacaacaccctaaac

SEQ ID NO: 14 - DNA, 20 bp, Artificial Sequence
  Primer
attttggttggatggttggt

SEQ ID NO: 15 - DNA, 20 bp, Artificial Sequence
  Primer
acaaccgaaaaatcccgaat

SEQ ID NO: 16 - DNA, 23 bp, Artificial Sequence
  Primer
tggggaagtagatagcgtagaaa

SEQ ID NO: 17 - DNA, 22 bp, Artificial Sequence
  Primer
ccctaaacaactccaccctacc

SEQ ID NO: 18 - DNA, 1000 bp, Homo sapiens
cggctcagcgcgggcctctttcactccgggccccagccttcccggtctcatttacagtcagaggcgctgaagctcagaaggtgcatgcgattcccctatttttctcccgtaatactgagactcagggaaatggaaataaactcttcttatttttcagacggaacccgggggctcaggcagaccctaagtcttgcccaaagcttcgcagtcgggcagcagtggagccaggatttggaagtaagaggacctgcggtgcttccctgcagtctcctgcgctgcgtcccacgccccgacaacacgcacgcaacacctacccctgtctcaccttgagcagatatcggtccaggatctcgaggccgcagctggagcagatgttcttgcctgcagacggcacggaggaggcggcagagggcggtgagcagacagatggcgtgctgggcgtacatggggaggcctgaccctcgtccttgtccagagctcctgccagggcctcggcgtcagactgagcctgcggcgggggagagaaggagaggagcgctgatccgggcacccagagttggggctgccttggagacaaatctggggcccccgagcaccaattgacaacgaaatctccccaggacaatgcgccgtagactgaaggacagggatggaagggcctattggagaggaagggacggccccaacttttaacacgcgcattctgggtgctgggagcggttaaagccaacattccctgccggaaaaccgaggctcagagaggggcggcgcccgccgggagtcccccagagcgtgagcagcagttcgtggccggaaaacctggccaggtccccagggccgggccgctggaggactgggcaccgcaggcagggccaaacggactcaccatggcgggcggcgcggtcccttcaagacagcgggtggtcgctttgcagccggaccctggctgggccatcacctgggggagggggggagggaacgcaggcggcggcggctgctg

SEQ ID NO: 19 - DNA, 1000 bp, Artificial Sequence
  Bisulfite modified sequence
cggtttagcgcgggttttttttatttcgggttttagttttttcggttttatttatagttagaggcgttgaagtttagaaggtgtatgcgatttttttatttttttttcgtaatattgagatttagggaaatggaaataaattttttttattttttagacggaattcgggggtttaggtagattttaagttttgtttaaagtttcgtagtcgggtagtagtggagttaggatttggaagtaagaggatttgcggtgtttttttgtagttttttgcgttgcgttttacgtttcgataatacgtacgtaatatttatttttgttttattttgagtagatatcggtttaggatttcgaggtcgtagttggagtagatgtttttgtttgtagacggtacggaggaggcggtagagggcggtgagtagatagatggcgtgttgggcgtatatggggaggtttgattttcgtttttgtttagagtttttgttagggtttcggcgttagattgagtttgcggcgggggagagaaggagaggagcgttgattcgggtatttagagttggggttgttttggagataaatttggggttttcgagtattaattgataacgaaatttttttaggataatgcgtcgtagattgaaggatagggatggaagggtttattggagaggaagggacggttttaatttttaatacgcgtattttgggtgttgggagcggttaaagttaatattttttgtcggaaaatcgaggtttagagaggggcggcgttcgtcgggagttttttagagcgtgagtagtagttcgtggtcggaaaatttggttaggtttttagggtcgggtcgttggaggattgggtatcgtaggtagggttaaacggatttattatggcgggcggcgcggtttttttaagatagcgggtggtcgttttgtagtcggattttggttgggttattatttgggggagggggggagggaacgtaggcggcggcggttgttg

SEQ ID NO: 20 - DNA, 20 bp, Artificial Sequence
  Primer
ggaattcgggggtttaggta

SEQ ID NO: 21 - DNA, 20 bp, Artificial Sequence
  Primer
acgctcctctccttctctcc

SEQ ID NO: 22 - DNA, 20 bp, Artificial Sequence
  Primer
gcgtatatggggaggtttga

SEQ ID NO: 23 - DNA, 23 bp, Artificial Sequence
  Primer
cctacgatacccaatcctccaac

SEQ ID NO: 24 - DNA, 4934 bp, Homo sapiens
ttgggagctcttggcctctctttgggcagagtgctccagtccacatcctaccagacccttgaccccgggcttcaggaaacagaagcctagagaacctcagcatgtgacccctccctgggccccagctccttagaggaaaattctgcctcccacgccaccattcacactctctccaggaagaacttcctcatagcagccacccagagcttgggctgcagtgcagtgcagacaatagtccttgcctggcactcgatcggctttttctttccctttttgtgggttttattatttttgttacaggtggggctgccagggatgggttatgcctgcagggggagaaatgatcagaagagccacaattctaccctcaacccagggaatgtgagcttttactgcctccattttcttaaccagaaatctggtttagttttgctttcagggctcttgtcattggaaaaagattagtgctcagaacctgcagcccagaaatggcccagtcccatccctccaggcaggctctgctcctcaagctgcgtggggaaatgccatcatgggcttgtgcccagcatccaccctgccaatgatgacaacagtgataatcgaggcattcataccaaccaccatttagggagcctttatagagacttccccccaacagtggcctctttggcagctctgggagacagccttggaaagggcgaactaagggccgagagaagcagcaacttgcgaaggtcactactggccagactgggcccagtctcctgcctcttcccacagtggctgcacaccctactctctaccccattgcagtaggaagagaactccagaataaggggaggctctgaacagtggagtcaggatccgtgttttgactagtttcaaggtctagtaaagcacagtatccaaggaggctcagtccagcatgggcatgggctagggctggagaagtagaaaagaaatttgggcaagtagtgaataagactgccaaatctattgttaggagagccaggagaggtgtcctagtggtgccttagtctatgggagagacccagacaccattctgctgggttcccaagctggcaccttaaatttagtagctgatgaatccaaggagcccccaagcatcatctggcatgtttttttgtatgtttgtttgtttgttttttgagacagagtctctctctgttacccaggctggagtgtagtgacgtgatcttggctcaccacaatgtccgcctcccaggttcaagtgattctcttgcctcagcctcctgagtagctgggactacaggcacgtgccaccacacctggctaagttttgtatttttagtagagatgggtttcaccatgttggccaggatggtctcgatctcctgaccttgtgatctaccagccttggcctcccaaagtgctgcgattataggtgtgagccaccacacccggcccatctggcatgttttataccaactggagtcaggcctagagcagggaggtggctagacagtgactgtaacctccatttcctgtcctggccaaaccaatactcagatgggacagtgagaggagacatcacactctggttacacaccattaacccctgtctgactacacacacacacacacacagccgtccagattcacaaatacgttcttatcacccccttcatccctcagtgcctgggtggaggcccttaagcaggaatgccccctcagaggagagggtgggagtggaatgatgggatttcagaatcccctcacctcaccctctcacctccagatcctagtccagatgtaatcctgtcctcaccactttgcgtgcacgtgcacgcacacacacatgcacacacacagtctcaacattcacatacagacacccactcttgacacccacacatgaactaaaagacaaacgttggtccataaaggaacacacatgctatctacatacccccaaccctgcccagtgatgctttccccagcactactgcacagatcccagacaatacagacacacaggcactccttgcacactgagtcctaggccacttggttgagctgcccccaccccaccccagctgcacactcttctacatccatcccataaaggaagagtccgtctctttaaccatgcttctcatggttttcagagtacccttgccccaaatgctgccaacctccccctagactcaagatcttctctttccagatcgctggaggctcaatgccagtgcacaggaggagatcgccttccaccatattccctgactcccagaggctctttctagggcaggggtctggccaggaagactgaagccatgacgtcttcctgtgcaaggataggaaagatgtctgtgggttggagggtagttatagggaagcagctttctccactttttgagaaagcacttgggaacaagcatccctctcaggaagtgattctccaccattctcctctgatcctataggcctggtggtccaagcctcaggccagttgatgcctcccactgggatcagcagacaaggccatgaccctgcagtgacctcgaccccataatcaggtcaggtccgggcttcatggctagaagcgaggcccaggcgagtggccaagacgcggcaggacaggaggatgcgcctggctgctctcggctgctggacgcggcctgcagtagggagagcgcagcccggggtaatcccactcagggctccgggtgcccagttctcccacttcccatcctggcggcctaaaggcaaggcctagaccctggagaggccaaagtcgggccaaaacaacagcgaagctgggactggaacaggcgctgggggcctgcgcatgaccccaggctcagaatgcgacactccagtgacgccggggaatgagttgagatggagccggagacagaggttggggttcaggacgaggcgcttgggtatgggatcggatctgagacggttgcggcgctggtgctttgggctggaccagactcgaacccagagcggagcgagggtcagagccagggcaagagtccgaagcccagctcagcgtccagcccgcgagagaccccaccatgaatgacgtccgagtagggttggaactcgaagcccgaacgcactggaaatgcagccggacgacggctgggatccgagtgggtccgggccagaaatggggacctcagagctccaccgaggcgctcgaggtcagaactgaagcctgagacttaggcaggacccgagacagagccagagacgataccgaaacccaatggaccgcgaggaccgaaggcagatccggggcgcaaacctgtgcaggcactggcgcgctgcctggagctctgggttcgcgccgctgagcgccggcaggttggaccagcgctgcgcgccggaagtgcactcgggacgccggggttctggaggagagaccacacgccgcaaaagtggcctccgaatgcgcccggggcctgccctcggcgacactgctggctcggcggccgcagcctacctgaagtagtccatcttgcagaagatctccttgttcttgatgtagcagctgttctgctgcctcagcgacgtgcgacacacggagcactcgaggcaccgcacgtgccagatgaggttgttgacctggggacggggcggggacggaggtcggctcagcgcgggcctctttcactccgggccccagccttcccggtctcatttacagtcagaggcgctgaagctcagaaggtgcatgcgattcccctatttttctcccgtaatactgagactcagggaaatggaaataaactcttcttatttttcagacggaacccgggggctcaggcagaccctaagtcttgcccaaagcttcgcagtcgggcagcagtggagccaggatttggaagtaagaggacctgcggtgcttccctgcagtctcctgcgctgcgtcccacgccccgacaacacgcacgcaacacctacccctgtctcaccttgagcagatatcggtccaggatctcgaggccgcagctggagcagatgttcttgcctgcagacggcacggaggaggcggcagagggcggtgagcagacagatggcgtgctgggcgtacatggggaggcctgaccctcgtccttgtccagagctcctgccagggcctcggcgtcagactgagcctgcggcgggggagagaaggagaggagcgctgatccgggcacccagagttggggctgccttggagacaaatctggggcccccgagcaccaattgacaacgaaatctccccaggacaatgcgccgtagactgaaggacagggatggaagggcctattggagaggaagggacggccccaacttttaacacgcgcattctgggtgctgggagcggttaaagccaacattccctgccggaaaaccgaggctcagagaggggcggcgcccgccgggagtcccccagagcgtgagcagcagttcgtggccggaaaacctggccaggtccccagggccgggccgctggaggactgggcaccgcaggcagggccaaacggactcaccatggcgggcggcgcggtcccttcaagacagcgggtggtcgctttgcagccggaccctggctgggccatcacctgggggagggggggagggaacgcaggcggcggcggctgctgaactggctccgcacagcctgagcccagcgcctccccgcgcctgttatataaaccggcgccgaacaatgagtcctaactttgtagtgggcatttaaagcccttccccacaaaagcggtgtctctctaatgaagcaatttgaatttggattggattttttccctctctctccccctctccctgcacttaacccgtggctcttgaagtaatcgcttagttcccttgcaatccaagcctctgaggggggaagaaaaacacgcgcacacacacacaaactacctgcaattaga

SEQ ID NO: 25 - DNA, 4934 bp, Artificial Sequence
  Bisulfite modified sequence
ttgggagtttttggtttttttttgggtagagtgttttagtttatattttattagatttttgatttcgggttttaggaaatagaagtttagagaattttagtatgtgatttttttttgggttttagttttttagaggaaaattttgttttttacgttattatttatattttttttaggaagaatttttttatagtagttatttagagtttgggttgtagtgtagtgtagataatagtttttgtttggtattcgatcggttttttttttttttttttgtgggttttattatttttgttataggtggggttgttagggatgggttatgtttgtagggggagaaatgattagaagagttataattttatttttaatttagggaatgtgagtttttattgtttttatttttttaattagaaatttggtttagttttgtttttagggtttttgttattggaaaaagattagtgtttagaatttgtagtttagaaatggtttagttttatttttttaggtaggttttgttttttaagttgcgtggggaaatgttattatgggtttgtgtttagtatttattttgttaatgatgataatagtgataatcgaggtatttatattaattattatttagggagtttttatagagatttttttttaatagtggtttttttggtagttttgggagatagttttggaaagggcgaattaagggtcgagagaagtagtaatttgcgaaggttattattggttagattgggtttagttttttgtttttttttatagtggttgtatattttattttttattttattgtagtaggaagagaattttagaataaggggaggttttgaatagtggagttaggattcgtgttttgattagttttaaggtttagtaaagtatagtatttaaggaggtttagtttagtatgggtatgggttagggttggagaagtagaaaagaaatttgggtaagtagtgaataagattgttaaatttattgttaggagagttaggagaggtgttttagtggtgttttagtttatgggagagatttagatattattttgttgggtttttaagttggtattttaaatttagtagttgatgaatttaaggagtttttaagtattatttggtatgtttttttgtatgtttgtttgtttgttttttgagatagagtttttttttgttatttaggttggagtgtagtgacgtgattttggtttattataatgttcgttttttaggtttaagtgatttttttgttttagttttttgagtagttgggattataggtacgtgttattatatttggttaagttttgtatttttagtagagatgggttttattatgttggttaggatggtttcgattttttgattttgtgatttattagttttggttttttaaagtgttgcgattataggtgtgagttattatattcggtttatttggtatgttttatattaattggagttaggtttagagtagggaggtggttagatagtgattgtaatttttattttttgttttggttaaattaatatttagatgggatagtgagaggagatattatattttggttatatattattaatttttgtttgattatatatatatatatatatagtcgtttagatttataaatacgtttttattattttttttattttttagtgtttgggtggaggtttttaagtaggaatgtttttttagaggagagggtgggagtggaatgatgggattttagaatttttttattttatttttttatttttagattttagtttagatgtaattttgtttttattattttgcgtgtacgtgtacgtatatatatatgtatatatatagttttaatatttatatatagatatttatttttgatatttatatatgaattaaaagataaacgttggtttataaaggaatatatatgttatttatatatttttaattttgtttagtgatgttttttttagtattattgtatagattttagataatatagatatataggtattttttgtatattgagttttaggttatttggttgagttgtttttattttattttagttgtatatttttttatatttattttataaaggaagagttcgtttttttaattatgttttttatggtttttagagtatttttgttttaaatgttgttaatttttttttagatttaagattttttttttttagatcgttggaggtttaatgttagtgtataggaggagatcgttttttattatattttttgatttttagaggttttttttagggtaggggtttggttaggaagattgaagttatgacgtttttttgtgtaaggataggaaagatgtttgtgggttggagggtagttatagggaagtagttttttttattttttgagaaagtatttgggaataagtattttttttaggaagtgattttttattatttttttttgattttataggtttggtggtttaagttttaggttagttgatgttttttattgggattagtagataaggttatgattttgtagtgatttcgattttataattaggttaggttcgggttttatggttagaagcgaggtttaggcgagtggttaagacgcggtaggataggaggatgcgtttggttgttttcggttgttggacgcggtttgtagtagggagagcgtagttcggggtaattttatttagggtttcgggtgtttagtttttttattttttattttggcggtttaaaggtaaggtttagattttggagaggttaaagtcgggttaaaataatagcgaagttgggattggaataggcgttgggggtttgcgtatgattttaggtttagaatgcgatattttagtgacgtcggggaatgagttgagatggagtcggagatagaggttggggtttaggacgaggcgtttgggtatgggatcggatttgagacggttgcggcgttggtgttttgggttggattagattcgaatttagagcggagcgagggttagagttagggtaagagttcgaagtttagtttagcgtttagttcgcgagagattttattatgaatgacgttcgagtagggttggaattcgaagttcgaacgtattggaaatgtagtcggacgacggttgggattcgagtgggttcgggttagaaatggggattttagagttttatcgaggcgttcgaggttagaattgaagtttgagatttaggtaggattcgagatagagttagagacgatatcgaaatttaatggatcgcgaggatcgaaggtagattcggggcgtaaatttgtgtaggtattggcgcgttgtttggagttttgggttcgcgtcgttgagcgtcggtaggttggattagcgttgcgcgtcggaagtgtattcgggacgtcggggttttggaggagagattatacgtcgtaaaagtggttttcgaatgcgttcggggtttgttttcggcgatattgttggttcggcggtcgtagtttatttgaagtagtttattttgtagaagatttttttgtttttgatgtagtagttgttttgttgttttagcgacgtgcgatatacggagtattcgaggtatcgtacgtgttagatgaggttgttgatttggggacggggcggggacggaggtcggtttagcgcgggttttttttatttcgggttttagttttttcggttttatttatagttagaggcgttgaagtttagaaggtgtatgcgatttttttatttttttttcgtaatattgagatttagggaaatggaaataaattttttttattttttagacggaattcgggggtttaggtagattttaagttttgtttaaagtttcgtagtcgggtagtagtggagttaggatttggaagtaagaggatttgcggtgtttttttgtagttttttgcgttgcgttttacgtttcgataatacgtacgtaatatttatttttgttttattttgagtagatatcggtttaggatttcgaggtcgtagttggagtagatgtttttgtttgtagacggtacggaggaggcggtagagggcggtgagtagatagatggcgtgttgggcgtatatggggaggtttgattttcgtttttgtttagagtttttgttagggtttcggcgttagattgagtttgcggcgggggagagaaggagaggagcgttgattcgggtatttagagttggggttgttttggagataaatttggggttttcgagtattaattgataacgaaatttttttaggataatgcgtcgtagattgaaggatagggatggaagggtttattggagaggaagggacggttttaatttttaatacgcgtattttgggtgttgggagcggttaaagttaatattttttgtcggaaaatcgaggtttagagaggggcggcgttcgtcgggagttttttagagcgtgagtagtagttcgtggtcggaaaatttggttaggtttttagggtcgggtcgttggaggattgggtatcgtaggtagggttaaacggatttattatggcgggcggcgcggtttttttaagatagcgggtggtcgttttgtagtcggattttggttgggttattatttgggggagggggggagggaacgtaggcggcggcggttgttgaattggtttcgtatagtttgagtttagcgttttttcgcgtttgttatataaatcggcgtcgaataatgagttttaattttgtagtgggtatttaaagtttttttttataaaagcggtgtttttttaatgaagtaatttgaatttggattggattttttttttttttttttttttttttttgtatttaattcgtggtttttgaagtaatcgtttagtttttttgtaatttaagtttttgaggggggaagaaaaatacgcgtatatatatataaattatttgtaattaga

SEQ ID NO: 26 - DNA, 22 bp, Artificial Sequence
  Primer
ttgggttgtagtgtagtgtaga

SEQ ID NO: 27 - DNA, 20 bp, Artificial Sequence
  Primer
aattcgccctttccaaaact

SEQ ID NO: 28 - DNA, 20 bp, Artificial Sequence
  Primer
gagagcgtagttcggggtaa

SEQ ID NO: 29 - DNA, 21 bp, Artificial Sequence
  Primer
tctcgcgaactaaacgctaaa

SEQ ID NO: 30 - DNA, 20 bp, Artificial Sequence
  Primer
aatttaatggatcgcgagga

SEQ ID NO: 31 - DNA, 20 bp, Artificial Sequence
  Primer
ccccaaatcaacaacctcat

SEQ ID NO: 32 - DNA, 20 bp, Artificial Sequence
  Primer
ggtgttgggagcggttaaag

SEQ ID NO: 33 - DNA, 20 bp, Artificial Sequence
  AAAACTCATTATTCGACGCC
aaaactcattattcgacgcc

SEQ ID NO: 34 - DNA, 804 bp, Homo sapiens
gagagatgggtacttcacgccacaaccccctcaaccaagaccacctccgcaaccacagcgggaaaagctaggggcaggcagcccacttttgcaattactctacattacagtgaactgcggtcctcacgtcttcttttcttcactccccttaacaaaataaaataaataatttcccttcctgcccacccacggttgcccaagaccaagtcttagaggcagccgggttccgatcaagtgctgcgaaacgacgcgtttgacagctgctctggagccgaggatcacaataagagaaggcgcgtgaggctccggcagcaaacaccagccaggcaggcagcggtaatcacagtgagcaaagttgaagactataaaagagccaggctaacgtgctgccgagcaggtgtcctggattttattgtagttgcaaatatgcttctggtgataagtatcgaaattattttttaagtgcgctggggaggagggaggtgcgggaattgagcctcctccgtttccagggagcctctgaaagtggggtggggggagatgaggcatgcacgcgaggcaaagacaaacaggaaagtgtctctatcgggaccaacgtgtgcgaccctacctgaaacggcaagcagaattcggtgttttcttccttttgccctgatcttgacgagataggaaacttgggagagagagagagagagagagagagagagagagagagagagagagagagagagagagagaccgagactgcagcagccaagagagcgtggagcgatgggctggtggaagccggagaggagcgctagg

SEQ ID NO: 35 - DNA, 804 bp, Artificial Sequence
  Bisulfite modified sequence
gagagatgggtattttacgttataatttttttaattaagattattttcgtaattatagcgggaaaagttaggggtaggtagtttatttttgtaattattttatattatagtgaattgcggtttttacgtttttttttttttattttttttaataaaataaaataaataatttttttttttgtttatttacggttgtttaagattaagttttagaggtagtcgggtttcgattaagtgttgcgaaacgacgcgtttgatagttgttttggagtcgaggattataataagagaaggcgcgtgaggtttcggtagtaaatattagttaggtaggtagcggtaattatagtgagtaaagttgaagattataaaagagttaggttaacgtgttgtcgagtaggtgttttggattttattgtagttgtaaatatgtttttggtgataagtatcgaaattattttttaagtgcgttggggaggagggaggtgcgggaattgagtttttttcgtttttagggagtttttgaaagtggggtggggggagatgaggtatgtacgcgaggtaaagataaataggaaagtgtttttatcgggattaacgtgtgcgattttatttgaaacggtaagtagaattcggtgtttttttttttttgttttgattttgacgagataggaaatttgggagagagagagagagagagagagagagagagagagagagagagagagagagagagagagatcgagattgtagtagttaagagagcgtggagcgatgggttggtggaagtcggagaggagcgttagg

SEQ ID NO: 36 - DNA, 21 bp, Artificial Sequence
  Primer
tcgggtttcgattaagtgttg

SEQ ID NO: 37 - DNA, 20 bp, Artificial Sequence
  Primer
tttcaaataaaatcgcacac